(19) United States
(12) Patent Application Publication
(10) Pub. No.: US 2024/0260910 A1
(43) Pub. Date: Aug. 8, 2024

(54) WIRELESS AND NONINVASIVE EPIDERMAL ELECTRONICS

(71) Applicant: NORTHWESTERN UNIVERSITY, Evanston, IL (US)

(72) Inventors: John A. Rogers, Wilmette, IL (US); Siddharth Krishnan, Evanston, IL (US); Tyler R. Ray, Evanston, IL (US); Amit B. Ayer, Evanston, IL (US); Philipp Gutruf, Evanston, IL (US); Jonathan T. Reeder, Evanston, IL (US); Kun Hyuck Lee, Morton Grove, IL (US); Chun-Ju Su, Evanston, IL (US)

(21) Appl. No.: 18/633,768

(22) Filed: Apr. 12, 2024

Related U.S. Application Data
(63) Continuation of application No. 17/568,791, filed on Jan. 5, 2022.
Application No. 17/568,791 is a division of application No. 17/043,111, filed on Sep. 29, 2020, now Pat. No. 11,259,754.
(60) Provisional application No. 62/791,390, filed on Jan. 11, 2019.
(60) Provisional application No. 62/650,826, filed on Mar. 30, 2018.

(51) Int. Cl. A61B 5/00; A61B 5/01

(52) U.S. Cl. CPC A61B 5/7278; A61B 5/01; A61B 2562/0271; A61B 2562/164; A61B 2562/18

(57) ABSTRACT

Hydrocephalus and shunt-related expenditures cost the US system over $2 billion dollars in annual expenses, with 125,000 shunt surgeries per year and an untreated mortality rate estimated at 50-60%. Existing diagnostics are expensive, inaccurate, and often harmful or invasive, and can lead to unnecessary admissions, further testing, or needless surgery. Collaborative efforts between Northwestern materials engineers headed by Dr. John Rogers alongside the leadership of neurological surgeons at Northwestern Memorial Hospital and Lurie Children's Hospital have produced and validated a noninvasive, thermal biosensor capable of diagnosing ventricular shunt malfunction.

### CROSS-REFERENCE TO RELATED APPLICATIONS

This application is a continuation application of U.S. patent application Ser. No. 17/568,791, filed Jan. 5, 2022, which is a divisional application of U.S. patent application Ser. No. 17/043,111, filed Sep. 29, 2020, now U.S. Pat. No. 11,259,754, which is a national stage entry of PCT Patent Application Serial No. PCT/US2019/025009, filed Mar. 29, 2019, which itself claims the benefit of and priority to U.S. Provisional Patent Application No. 62/650,826 filed Mar. 30, 2018, and U.S. Provisional Patent Application No. 62/791,390, filed Jan. 11, 2019, each of which is specifically incorporated by reference in its entirety to the extent not inconsistent herewith.

### STATEMENT REGARDING FEDERALLY SPONSORED RESEARCH OR DEVELOPMENT

Not applicable.

### BACKGROUND OF INVENTION

Hydrocephalus and shunt-related expenditures cost the US system over $2 billion dollars in annual expenses, with 125,000 shunt surgeries per year and an untreated mortality rate estimated at 50-60%. Existing diagnostics are expensive, inaccurate, and often harmful or invasive, and can lead to unnecessary admissions, further testing, or needless surgery. To address these issues, provided herein is a noninvasive, thermal biosensor capable of diagnosing ventricular shunt malfunction.

Hydrocephalus is a common and costly condition caused by the accumulation of cerebrospinal fluid in the brain. It occurs in 1-5 of every 1000 live births, and over 70,000 patients are admitted and diagnosed yearly in the United States. Cerebrospinal fluid (CSF) is produced within the ventricles of the brain and is responsible for its nourishment and protection, but dysfunction in its drainage or reabsorption can lead to devastating neurological complications. Symptoms may include headache, lethargy, seizures, coma, or death, and untreated hydrocephalus has a mortality rate estimated at 50-60%. Pediatric hydrocephalus accounts for 38% of patients, and related surgeries are the most common neurosurgical procedures performed in children. Adult hydrocephalus can be seen as the sequelae to many conditions (such as tumors, trauma and infection) or in normal pressure hydrocephalus (NPH), an increasingly diagnosed condition which currently affects 375,000 patients in the United States. Often underdiagnosed or misdiagnosed for dementia and with 25% of those diagnosed shunted, this represents a growing segment of hydrocephalus related care. The mainstay of treatment for hydrocephalus involves the use of CSF diversion accomplished through devices known as ventricular shunts. These surgically implanted devices consist of a catheter draining from the ventricle to a distal site (such as the peritoneum, pleural cavity, or right atrium of the heart) and regulated by a valve. Unfortunately, shunts have extremely high failure rates; most children undergo 2-3 surgical revisions before adulthood and 30% are expected to have at least 1 revision operation in the first year, with rates approaching 98% by 10 years in some studies.

ShuntCheck® utilizes an ice-pack based thermal cooling system connected to a Windows PC DAQ to address a need for shunt monitoring. That technology, however, is cumbersome and time-consuming. The device's cumbersome, multi-step protocol; equivocal or negative past clinical studies; and need for ice-pack cooling have limited its acceptance. Additionally, patient discomfort due to prolonged skin cooling (detrimental for pediatric diagnostics) and absence of chronic monitoring further limits its diagnostic relevance. Accordingly, there is a need for a wireless noninvasive shunt diagnostic, that is conformable to skin and has epidermal-like mechanical properties.

### SUMMARY OF THE INVENTION

The devices presented herein provide a platform for measuring flow in subdermal conduits and are advantageously non-invasive and rapid, while preserving a high level of accuracy. The devices may be conformal to skin and wireless without a need for hard-wire connection to bulky external components, such as controllers, digital monitors and power supplies. In this manner, the device is painless and non-obtrusive to a patient, akin to wearing an adhesive bandage.

One particular application for any of the devices and methods described herein is detection of shunt malfunction, specifically ventricular shunts. Extended use can capture occult malfunction, akin to a holter monitor for cerebrospinal fluid.

Provided herein is a soft, wireless, noninvasive, non-surgical, skin-mounted device for the continuous measurement of fluid flow in a subdermal conduit, such as shunt-based CSF flow. The epidermal device exploits the precise measurement of thermal transport to characterize CSF flow in underlaid shunts. The device platform is ultrathin (<100 μm), soft (70 kPa), flexible resulting in a continuously wearable device mechanically invisible to the wearer. Similar in size to a Band-Aid® adhesive bandage, the device is composed primarily of soft, silicone rubber (no hard edges) and transmits recorded data wirelessly via Bluetooth to a companion mobile app. Patient data and in vitro tests confirm device efficacy in producing clinical-quality data suitable for shunt malfunction diagnostics. To assist in handling durability and device placement, a carrier substrate having an open passage through a central portion of the carrier substrate, may be provided around the active sensors region, where intimate conformal contact is desired. The carrier substrate may have a relatively larger mechanical parameter compared to the substrate that supports the sensors, such as being less flexible, elastic and/or soft, so that the device can be handled in a manner similar to an adhesive bandage (e.g., does not tear during application and use) but without sacrificing conformability and patient comfort.

The devices and methods described herein provide a fundamental platform for measuring flow in a wide range of artificial and natural flow conduits. Examples include, but are not limited to, catheters, stents and blood vessels.

The claims appended herein are specifically incorporated by reference herein and form part of the application.

Provided herein are various conformable devices capable of reliably, accurately, and continuously measuring subdermal fluid flow, including in a conduit. Various active components are supported by a substrate, such as a substrate that is characterized as soft, stretchable and flexible. A thermal actuator, an upstream temperature sensor and a downstream temperature sensor is supported by the substrate. A microprocessor is in electronic communication with the temperature sensors and other relevant components, such as the actuator, to calculate subdermal fluid flow from the measured upstream and downstream temperatures. Such a configuration maximizes patient comfort during use, facilitating long-term monitoring of fluid flow. Any of the devices may be wireless, further facilitating low patient impact monitoring, including without a need for hard-wire connections. In this manner, the patient may even return to home, without adversely impacting monitoring.

To further increase the accuracy and reliability of fluid flow measurement, any of the devices described herein may have additional temperature sensors. The position of those sensors may be described relative to a notional line (e.g., imaginary) line that is formed by drawing a line over spatially aligned upstream sensor, actuator, and downstream sensor. The additional sensors function as reference sensors and assist with determining various skin properties and related convection-type properties, for example, useful in determining fluid flow rate and the effect of the actuator independent of flow. Preferably, at least one sensor is positioned so that a temperature reading is obtained that is independent of whether or not the thermal actuator is actuated.

Also provided is a method of determining fluid flow in a sub-dermal conduit by any of the devices described herein. For example, a device is conformally mounted to skin that overlays the sub-dermal conduit. The thermal actuator is actuated to heat the underlying skin and sub-dermal conduit. Temperature upstream and downstream of the thermal actuator is measured wherein the sensors measuring the temperature are spatially aligned with the conduit. The microprocessor processes the measured temperatures to determine a flow-rate in the sub-dermal conduit. The determined flow-rate is transmitted to a display on a handheld device or computer. Preferably the method is wireless and data generated from the conformal device is wirelessly provided to the handheld device or computer for real-time monitoring.

Further, described herein are resistive heating thermal actuators utilizing an array of resistor components that provide heating upon application of an electrical current or potential. These thermal actuators may utilize various resistors known in the art, in some cases arranged in an array (e.g. circular, square, linear) to precisely provide thermal energy to allow for various measurements provided herein.

Additionally, various methods for increasing the signal to noise ratio of thermal measurements are also provided. For example, additional layers that partially or fully encapsulate and insulate various components may enhance the ability to isolate thermal energy provided by the various actuation means and increase the accuracy and reliability of sensing. Conductive layers may also be provided to increase the efficacy of thermal actuation and thermal sensing by providing a conduit for thermal energy to be directed to or received from the skin. Various discontinuous thermal conductive layers may further increase the signal to noise ratio for a variety of measurements, including thermal sensing.

Also described herein are non-electronic methods for thermal sensing. For example, optical measurements via a thermal imaging system or thermochromatic dyes may be utilized in place of electronic thermal sensors to determine tissue characteristic or parameters, including subdermal fluid flow.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIGS. 1A-1E. Soft, skin-mounted wearable device for noninvasive, continuous, or intermittent measurement of flow through cerebrospinal shunts for evaluation of shunt functioning. FIG. 1A. Exploded view illustration of 100-sensor device that incorporates a central thermal actuator, placed over skin with an underlying shunt catheter (“conduit”). PI=polyimide FIG. 1B. Optical micrograph of device, illustrating sensors, including a plurality a plurality of individual resistive sensors arranged in an array, and central thermal actuator. The close up panels illustrate the stretchable, serpentine interconnects to facilitate conformability and individual resistive temperature sensors. FIG. 1C. Infrared (IR) thermographs illustrating addressing of an individual sensor (left), and thermal actuation from central heater with 1.8 mW/mm² actuation power. FIG. 1D. Optical images of device on neck, over location of shunt, under different deformation modes. FIG. 1E. IR thermographs with color and contrast enhancement showing thermal isotropy in the absence of flow (top) and anisotropy in the presence of flow (bottom), with flow going towards the right of the page.

FIGS. 2A-2E. Flow visualization and measurement from ESA (epidermal square array). FIG. 2A. Spatially precise schematic map of 100 sensor device with tube position overlay and upstream (Tᵤ) and downstream (Td) temperatures shown. FIG. 2B. Baseline-subtracted temperature differentials of 4 sensor pairs as shown by the color coding in FIG. 2A. FIG. 2C. Principal components analysis (PCA) biplot (principle component 1 and 2) of baseline-subtracted differentials between a selected Tᵤ sensor (two sensors, each indicated in subfigure) and each Td sensor. Clustering occurs for the following cases: no flow and no actuation; no flow with actuation at 1.8 mW/mm⁻²; Actuation at 1.8 mW mm⁻² and flow at 0.02 mL min⁻¹; Actuation at 1.8 mW/mm² and flow at 2 mL min⁻¹. Vectors correspond to selected Td sensors correlated positively (red) and negatively (blue) with flow. FIG. 2D. Flow chart detailing the process of transforming raw ESA sensor data to spatially precise temperature maps. FIG. 2E. Thermographs from IR imaging (top) and ESA-generated temperature maps (bottom), in the absence (left) and presence (right) of 0.02 mL min⁻¹ flow (flow from right to left) with actuation at 1.8 mW/mm⁻².

FIGS. 3A-3L. Systemic characterization of effects of geometry, thermal properties, flow rates. FIG. 3A. Optical image of epidermal linear array (ELA) overlaid with illustration of catheter and blood vessel (top) and schematic illustration of benchtop system illustrating key features, including thermal properties of skin phantom, blood flow (Q_{blood}), CSF flow (Q_{flow}) and skin thickness (hₛₖᵢₙ). FIG. 3B. Raw transient temperature data after the onset of heating for actuator (blue curve), downstream sensor (black curve) and upstream sensor (red curve) under 4 values of Q_{flow}—0 mL min⁻¹ (unshaded region), 0.05 ml min⁻¹ (blue shaded region), 0.1 mL min⁻¹ (gray shaded region) and 0.5 ml min⁻¹ (red shaded region). FIG. 3C. Tₛₑₙₛₒᵣₛ/T_{actuator} for upstream (red) and downstream (black) sensors across a range of flow rates from 0.01 ml min⁻¹ to 0.1 mL min⁻¹. FIG. 3D. ΔTₛₑₙₛₒᵣₛ/T_{actuator}=(T_{downstream}−Tᵤₚₛₜᵣₑₐₘ)/T_{actuator} for a range of Q_{flow} from 0.01 ml min to 0.1 ml min for three anatomically relevant values of hₛₖᵢₙ, 1.1 mm (black curve), 1.7 mm (red curve) and 2.1 mm (blue curve). FIG. 3E. T̅ₛₑₙₛₒᵣₛ=(T_{downstream}+Tᵤₚₛₜᵣₑₐₘ)/2T_{actuator} for the same Q_{flow} and hₛₖᵢₙ values as FIG. 3D. FIG. 3F. Ratio between signal (ΔTₛₑₙₛₒᵣₛ/T_{actuator}) and noise (standard deviation, σ) measured for Q_{flow}=0.1 mL min over a 60 s sampling window, at a sampling frequency of 5 Hz, as a function of normalized actuator power for three different values of hₛₖᵢₙ, 1.1 mm (black curve), 1.7 mm (red curve) and 2.1 mm (blue curve). FIGS. 3G and 3E. (ΔTₛₑₙₛₒᵣₛ/T_{actuator}) (FIG. 3G) and (T_{downstream}+Tᵤₚₛₜᵣₑₐₘ)/2T_{actuator} (FIG. 3E) measured in the presence of phantom blood flowing through adjacent tubes in co-flow (+x) and counter-flow (−x) configurations, for two values of hₛₖᵢₙ, 1.1 mm (black curve) and 2.1 mm (blue curve). FIG. 3H is a plot of the ratio of sensor to actuator temperature as a function of blood flow. FIG. 3I. Experimental data (solid lines) and analytical fits (dashed lines) for T_{actuator} as a function of time for Q_{flow}=0 for two different skin phantoms, Sylgard 184 (black curve) and Syl 170 (gray curve) to simulate and measure skin thermal properties.

FIGS. 3J-3K. ΔTₛₑₙₛₒᵣₛ/T_{actuator}) (FIG. 3J) and (T_{downstream}+Tᵤₚₛₜᵣₑₐₘ)/2T_{actuator} (FIG. 3K) measured for the two skin phantom materials. FIG. 3L In vitro experimental measurements of ΔTₛₑₙₛₒᵣₛ/T_{actuator} for hₛₖᵢₙ (1.1, 1.7, 2.1, and 6.0 mm for four flowrates) and for Q_{flow} (0 ml/min (black curve), 0.05 ml/min (red curve), 0.1 ml/min (blue curve), and 0.5 ml/min (purple curve)).

FIG. 4A-4H. Wireless device, including Bluetooth communication with a portable device. FIG. 4B is an image of a fully assembled, integrated wireless ELA showing soft, conformal sensing/actuating components, flex-PCB (Cu/PI/Cu), and surface-mounted electronic components, including battery and wireless communication components. PDMS, polydimethylsiloxane. FIG. 4C is an image of device bending, showing flexibility. FIG. 4D is an image of a device mounted on the skin using medical-grade, acrylate-based pressure-sensitive adhesive. FIG. 4E. Raw sensor readout in measured bits from an 8-bit ADC during actuation and flow. FIG. 4F. IR-measured temperature rise due to 3.6-mW actuation on the phantom shunt assembly. FIG. 4G Calibration curve to measure raw 8-bit, 3-V ADC values (left) and associated voltages (right) to temperatures via calibration. FIG. 4H. Difference in Tᵤₚₛₜᵣₑₐₘ and T_{downstream} acquired wirelessly as a function of time for two different flows, Q=0.05 mL/min and Q=0.13 mL/min. All data are collected on a skin phantom.

FIG. 5A-5J. Patient trials. FIG. 5A. Exploded view illustration of ELA used in hospital setting, with elastomeric handling frame and adhesive. FIG. 5B. Illustration (left) and image (right) of on-shunt and off shunt ELA positioning on patient, with representative Doppler ultrasound image (inset) of catheter under skin at on-shunt location. FIG. 5C. IR images at on-shunt (top) and off shunt (bottom) indicating total local temperature rise due to actuator, and characteristic tear-drop shaped heat distribution caused by presence of flow. FIG. 5D. Representative transient T_{actuator} measurement on off-shunt location, and transient plane source (TPS) curve fit to yield skin thermal properties. FIGS. 5E-5F. Computed values of kₛₖᵢₙ (FIG. 5E) and αₛₖᵢₙ (FIG. 5F) for each patient. FIGS. 5G-5H. Representative T_{actuator} (blue curve), Tᵤₚₛₜᵣₑₐₘ (black curve) and T_{downstream} (red curve) for off-shunt location with no anisotropy (FIG. 5G) and on-shunt location with significant anisotropy (FIG. 5H). FIG. 5I. ΔTₛₑₙₛₒᵣₛ/T_{actuator} measured for each patient, at off-shunt and on-shunt locations, with error bars representing SDs across a 100-sample window. FIG. 5J is a plot of the computed mean of ΔTₛₑₙₛₒᵣₛ/ΔT_{actuator} on n=5 patients with clinically or surgically confirmed flow on off-shunt and on-shunt locations, with error bars representing SD. Statistical analysis was performed using a paired t test (n=5) for cases with confirmed flow over on-shunt and off-shunt locations. Individual patient-level data are summarized as Patient #(ΔTₛₑₙₛₒᵣₛ/T_{actuator} On Shunt and ΔTₛₑₙₛₒᵣₛ/T_{actuator} Off Shunt): Patient 1 (0.209339 and 0.00205); Patient 2 (0.0518 and 0.0084); Patient 3 (0.09503 and −0.00597); Patient 4 (0.100991 and 0.0061); Patient 5 (0.1392 and 0.000963).

FIGS. 6A-6D. Case study of patient with shunt malfunction. FIG. 6A. X-Ray and radionuclide tracer showing kinking and occlusion of catheter. FIG. 6B. Optical image of patient's peritoneal cavity immediately after surgery showing flow in repaired shunt. FIG. 6C. X-ray and radionuclide tracer confirming working of repaired shunt. FIG. 6D. ΔTₛₑₙₛₒᵣₛ/T_{actuator} measured by ELA before and after revision, at locations over (on) and adjacent to (off) shunt, before and after revision, confirming results from X-Ray and Radionuclide tracer.

FIGS. 7A-7D. Computation of flow rates. FIG. 7A. FEA-computed family of curves for different skin thicknesses of ΔTₛₑₙₛₒᵣₛ/T_{actuator} with data measured in-vivo from each patient overlaid. FIG. 7B. Computed curves for Tₛₑₙₛₒᵣₛ/T_{actuator} for different skin thicknesses. FIG. 7C. Computed flow rates from iteratively solving for both ΔTₛₑₙₛₒᵣₛ/ΔT_{actuator} and Tₛₑₙₛₒᵣₛ/T_{actuator} with error bars representing average differences in the individual values yielded by the two curves. FIG. 7D. FEA-computed values of ΔTₛₑₙₛₒᵣₛ/ΔT_{actuator} and Tₛₑₙₛₒᵣₛ/T_{actuator} using values of hₛₖᵢₙ=1.5 mm (acquired from CT imaging) and kₛₖᵢₙ=0.29 W m⁻¹ K⁻¹ and αₛₖᵢₙ=0.091 mm² s⁻¹ acquired in vivo from a patient as shown previously, overlaid with experimentally measured points from the same patient, yielding a flow rate of 0.1 mL/min.

FIGS. 8A-8B. Current pathways through resistive arrays. FIG. 8A. IR image (top) and simulations of ESA with single sensor addressed, showing currents through same input line (row) and output line (column). FIG. 8B. Same as FIG. 8A, but for a non-square array (16×6), showing large power dissipation through non-addressed sensors in same output line (spoke).

FIG. 9. Schematic illustration of data acquisition and control system for 100 sensor array.

FIG. 10. Heat map with each pixel corresponding to a residual (R²) value computed for each element in 10×10 array from linearly fitting Iₘₑₐₛ to temperature for calibration.

FIG. 11. Illustration of steps to convert measured current values to heat map, with steps corresponding to the images of FIG. 2D. Flow visualization and measurement from ESA. Top panel: Example of raw (resistance) ESA data. Second panel: Transformation of raw ESA data to calibrated temperatures via a calibration matrix specific to each ESA. Third panel: temperature differentials resulting from the removal of isotropic heat transfer effects from the thermal actuator via baseline subtraction. Bottom panel: ESA temperature map obtained from temperature differential map of preceding panel by meshed bicubic interpolation.

FIGS. 12A-12C. Flow visualization and measurement from ESA. FIG. 12A: Spatially precise schematic map of 100 sensor device with tube position overlay and upstream (U) and downstream (D) temperatures shown. FIG. 12B: Principal components analysis (PCA) biplot (principle component 1 and 2) of baseline-subtracted differentials between a selected U sensor (two sensors, each indicated in subfigure) and each D sensor. Clustering occurs for the following cases: no flow and no actuation; no flow with actuation at 1.8 mW/mm⁻²; Actuation at 1.8 mWmm⁻² and flow at 0.02 mL min⁻¹.

FIG. 12C. PCA biplots for five (1-5) sensors (identified in FIG. 12A) illustrating the identification of the sensors aligned with the flow direction regardless of selected sensor (red vector). When a PCA model is applied to the aligned data (used to generate temperature maps), PC1 correlates to presence/absence of flow and PC2 corresponds to thermal actuation state (on/off).

FIGS. 13A-13C. Benchtop flow system. FIG. 13A. Optical image of benchtop flow phantom with embedded shunt. FIG. 13B. Optical micrograph of cross section and isometric views showing catheter geometry and hₛₖᵢₙ. FIG. 13C. Sensor laminated onto the free surface of the assembly.

FIG. 14. Finite element simulations of dimensionless scaling parameters illustrating time evolution of heat through skin, as a measure of depth penetration, with experimentally measured numbers from the system overlaid.

FIG. 15. Experimental and simulated transient responses of ΔTₛₑₙₛₒᵣₛ/T_{actuator} for three different values of hₛₖᵢₙ for Q_{flow}=0.13 mL min⁻¹ as a demonstration of an alternative method to quantify skin thickness, with data showing relationship between the time constant (T=time taken to reach 63.7% of steady-state value) and hₛₖᵢₙ (inset).

FIG. 16. T_{actuator} and Tₛₑₙₛₒᵣₛ as a function of power level for Q_{flow}=0.13 mL min⁻¹ on Sylgard 184 skin phantom.

FIG. 17. Illustration of covered and uncovered (encapsulated) actuator measurements (left) to yield transient rise curves for fitting the value of H_{conv} (right).

FIGS. 18A-18C. FIG. 18A. Illustration and experimental data showing the effect of (FIG. 18B) rotational and (FIG. 18C) translational mispositioning on measured values of ΔTₛₑₙₛₒᵣₛ/T_{actuator} (black curve) and Tₛₑₙₛₒᵣₛ/T_{actuator} (red curve).

FIGS. 19A-19DF. DC Noise sources. FIG. 19A. Simplified schematic of data acquisition system for ELA. FIG. 19B. Standard deviations as a function of sampling window for resistances measured by ELA (black), a commercial sensor connected via ACF cable (blue) and a commercial resistor connected via soldered lead wires (red). FIG. 19C. Standard deviation as a function of sampling window for actuator output power. FIG. 19D. Standard deviation for measured ΔTₛₑₙₛₒᵣₛ/T_{actuator} as a function of sampling window for Q_{flow}=0.13 mL min⁻¹ on benchtop system, when covered by an enclosure (black) and uncovered (red). FIG. 19E. High Frequency Noise. Panel A. Schematic illustration of experimental system. Panel B. Fourier transform of resistance measured at 20 kHz. Panel C. S/N, computed as the average of 5 successive resistance measurements divided by their standard deviation as a function of number of samples (N) and sampling window (time, ns). Panel D. Experimental data and linear fit for S/N as a function of N. FIG. 19F. S15. In-vivo noise A. Optical images illustrating no deformation (left) and extreme deformation (right) of sensor on skin. B-D. Temperature fluctuations measured as a function of time (B), frequency (C) and as a normalized power spectral density (D) on a stationary subject. E-G Same as B-D on a vigorously moving subject.

FIG. 20. Optical images of elastomeric adhesive with tape frame on wrist illustrating conformal contact during extreme deformation.

FIGS. 21A-21B. In-vivo T_{actuator} (blue curve), Tᵤₚₛₜᵣₑₐₘ (black curve) and T_{downstream} (red curve) measurements as a function of time over on-shunt locations with low anisotropy (FIG. 21A) and after stimulating flow by pressing the regulating valve (FIG. 21B).

FIG. 22. T_{actuator} measurements on external ventricular drain as flow is varied by raising height of reservoir bag (not shown), thereby changing differential pressure.

FIG. 23. Representative CT image of skin thickness over superficial catheter location over clavicle.

FIG. 24. Schematic illustration of relevant parameters.

FIG. 25. Flow-chart summary of flow rate determination using any of the devices described herein.

FIG. 26. Illustration of carrier and handling layer, with the device peeled back and away from the rigid handling layer of glass.

FIG. 27. Skin-safe, silicone adhesive, with active sensing portion of device able to maintain. conformal contact with skin, with delamination confined to edge handling substrate that surrounds the active sensing portion.

FIG. 28. Another illustration showing the handling substrate with an opening where the active sensing portion of device may be positioned.

FIG. 29. provides an overview of the sensing platform technology, including hardware and software.

FIG. 30. illustrates an example sensor design for commercial, surface mounted temperature sensors.

FIG. 31. provides an example of a flexible printed circuit board (PCT) based flow sensor including thermal actuation by an array of resistive elements.

FIG. 32. provides an analog design of circuits described herein.

FIG. 33. illustrates the use of thermochromatic dyes arranged in an array to determine subdermal fluid flow.

FIG. 34. provides an example of a thermal imaging approach and currently available inexpensive thermal imaging devices.

FIG. 35. provides an example of in vitro testing of a surface-mount device ad described herein without foam insulation.

FIG. 36. provides an example of in vitro testing of a surface-mount device ad described herein with a foam insulation layer, illustrating the increase in signal to noise ratio provided by insulation.

FIG. 37. provides in vitro testing of a surface-mount device with foam insulation across flow rates and relevant skin thicknesses, including a discontinuous thermally conductive layer positioned proximate to the thermal actuator and sensors.

FIG. 38. provides an example sensor integrated with packaging and encapsulation for thermal insulation.

FIG. 39. shows an example device with encapsulation removed to expose and illustrate the various components as described herein.

FIG. 40. provides both benchtop and on-body sensing results of an example device.

FIG. 41. illustrates the ability of a sensing device to measure change in temperature when positioned over a shunt as an in vivo example.

FIG. 42. illustrates the use of a device with multiple sensors and provides an example circuit diagram.

FIG. 43. illustrates the increase in rotational tolerance for 4-sensor device.

FIG. 44. illustrates the increase in translational tolerance for 4-sensor device.

FIG. 45. provides example hardware for wireless, inductive power coupling for recharging and BLE wake-up.

FIG. 46. provides an example software interface.

FIG. 47. provides a summary of clinical results.

FIG. 48. provides an example of a clinical protocol that may be useful to ensure accurate application of the devices described herein.

FIG. 49. provides an example of a clinical checklist that may be useful to ensure accurate application of the devices described herein.

FIG. 50. provides an example schematic of a device as described herein utilizing an array of resistors to provide thermal actuation.

FIG. 51 provides an example cross-sectional schematic of a device incorporating an insulating layer and a discontinuous thermally conductive layer.

FIGS. 52A-52C illustrate the effect of altered intersensor distances (L). FIG. 52A is a schematic illustration showing positions of actuator and upstream and downstream temperature sensors relative to underlying catheter. FIG. 52B FEA simulation of ΔTₛₑₙₛₒᵣₛ/T_{actuator} as a function of L, for hₛₖᵢₙ=0.5 mm, 1.0 mm, 1.5 mm, 2.0 mm, with the effect of 15% strain resulting in an altered inter-sensor positional uncertainty of +0.375 mm, as shown by the rectangular bar. FIG. 52C FEA simulation of T̅ₛₑₙₛₒᵣₛ/T_{actuator} as a function of L, for hₛₖᵢₙ=0.5 mm, 1.0 mm, 1.5 mm, 2.0 mm, with the effect of 15% strain resulting in an altered inter-sensor positional uncertainty of ±0.375 mm, as shown by the rectangular bar.

FIGS. 53A-53D. Miniaturized, soft wireless flow sensor based on commercial components. FIG. 53A. Exploded view schematic of key device layers. FIG. 53B. Optical images of packaged, encapsulated device twisting and bending. FIG. 53C. Optical image of device mounted on neck of patient. FIG. 53D. Screenshot of software application on tablet computer showing data readout, pairing and options for on-demand thermal actuation.

FIGS. 54A-54F. Benchtop flow characterization using platform. FIG. 54A. Exploded view schematic of sensors and actuators with overlaid foam layer over shunt embedded in silicone skin phantom. FIG. 54B. Infrared (IR) thermograph of actuator dissipating thermal power at 1.2 mW/mm². FIG. 54C. Upstream (gray) and downstream (red) temperature readout after actuation, and after during flow respectively, showing the bifurcation of the traces (ΔT) and the reduced overall average temperature (T_{avg}), respectively, after the onset of flow. FIG. 54D. ΔT as a function of time before and after the onset of flow. FIG. 54E. ΔT as a function of flow rate for a range of physiologically relevant skin thicknesses, from 0.7 mm to 4 mm. FIG. 54F. T_{avg} as a function of flow rate for a range of physiologically relevant skin thicknesses.

FIGS. 55A-55B. Patient studies on adults. FIG. 55A. Optical image of wireless sensor over shunt on representative patient, without smartphone readout. FIG. 55B. ΔT for cases with confirmed flow, no flow/irregular flow and off shunt locations, with error bars representing S.D.

FIG. 56. Spatial and Temporal Precision of negative temperature coefficient temperature sensors (NTCs).

FIG. 57. Stability of temperature sensors, with measured temperature from two temperature sensors as a function of time.

FIG. 58. Unpackaged circuit layout providing various electronic components on-board the device, including for power, wireless communication and circuitry to control and measure.

FIG. 59. Analog front end and wireless temperature sensing precision. The temperature sensors show high linearity over a range of biologically-relevant skin temperatures.

FIGS. 60A-60D. Power-saving switch feature.

FIG. 61. Molding and packaging process that can be used to make any of the devices of the instant invention.

FIG. 62. Device configured to have rotational tolerance by a 4-sensor device. The plots are for a device aligned and for various rotations of 22.5, 45 and 90 degree rotation.

FIG. 63. Device configured to have translational tolerance by a 4-sensor device. The plots are for a device aligned and for various translational offsets of 2 mm, 5 mm and complete misalignment.

FIG. 64. Effect of foam insulation on temperature sensors.

FIG. 65. Applicability to blood vessels, with the left panels for a device that is not over a blood vessel and the right panels for a device over a vein.

FIG. 66. Representative clinical images of a device positioned on and off shunt.

FIG. 67. Schematic illustration of a device.

### DETAILED DESCRIPTION OF THE INVENTION

In general, the terms and phrases used herein have their art-recognized meaning, which can be found by reference to standard texts, journal references and contexts known to those skilled in the art. The following definitions are provided to clarify their specific use in the context of the invention.

“Soft” refers to a material that may be comfortably positioned against the skin without discomfort or irritation to the underlying skin by the material itself deforming to conform to the skin without unduly exerting force on the underlying skin with corresponding device-generated skin deformation. Softness/hardness may be optionally quantified, such as in terms of durometer, or a material's resistance to deformation. For example, the substrate may be characterized in terms of a Shore 00 hardness scale, such as a Shore 00 that is less than 80. Soft may also be characterized in terms of a modulus, such as a Young's modulus that is less than or equal to 100 kPa.

“Stretchable” refers to a material's ability to undergo reversible deformation under an applied strain. This may be characterized by a Young's modulus (stress/strain). A bulk or effective Young's modulus refers to a composite material formed from materials having different Young's modulus, so that the bulk or effective Young's modulus is influenced by each of the different materials and provides an overall device-level modulus.

“Flexible” refers to a material's ability to undergo a bending with fracture or permanent deformation, and may be described in terms of a bending modulus.

Any of the devices may be described herein as being “mechanically matched” to skin, specifically the skin over which the device will rest. This matching of device to skin refers to a conformable interface, for example, useful for establishing conformal contact with the surface of the tissue. Devices and methods may incorporate mechanically functional substrates comprising soft materials, for example exhibiting flexibility and/or stretchability, such as polymeric and/or elastomeric materials. A mechanically matched substrate may have a modulus less than or equal to 100 MPa, less than or equal to 10 MPa, less than or equal to 1 MPa. A mechanically matched substrate may have a thickness less than or equal to 0.5 mm, and optionally for some embodiments, less than or equal to 1 cm, and optionally for some embodiments, less than or equal to 3 mm. A mechanically matched substrate may have a bending stiffness less than or equal to 1 nN m, optionally less than or equal to 0.5 nN m.

A mechanically matched device, and more particularly a substrate is characterized by one or more mechanical properties and/or physical properties that are within a specified factor of the same parameter for an epidermal layer of the skin, such as a factor of 10 or a factor of 2. For example, a substrate may have a Young's Modulus or thickness that is within a factor of 20, or optionally for some applications within a factor of 10, or optionally for some applications within a factor of 2, of a tissue, such as an epidermal layer of the skin, at the interface with a device of the present invention. A mechanically matched substrate may have a mass or modulus that is equal to or lower than that of skin.

“Encapsulate” refers to the orientation of one structure such that it is at least partially, and in some cases completely, surrounded by one or more other structures, such as a substrate, adhesive layer or encapsulating layer. “Partially encapsulated” refers to the orientation of one structure such that it is partially surrounded by one or more other structures, for example, wherein 30%, or optionally 50%, or optionally 90% of the external surface of the structure is surrounded by one or more structures. “Completely encapsulated” refers to the orientation of one structure such that it is completely surrounded by one or more other structures.

“Polymer” refers to a macromolecule composed of repeating structural units connected by covalent chemical bonds or the polymerization product of one or more monomers, often characterized by a high molecular weight. The term polymer includes homopolymers, or polymers consisting essentially of a single repeating monomer subunit. The term polymer also includes copolymers, or polymers consisting essentially of two or more monomer subunits, such as random, block, alternating, segmented, grafted, tapered and other copolymers. Useful polymers include organic polymers or inorganic polymers that may be in amorphous, semi-amorphous, crystalline or partially crystalline states. Crosslinked polymers having linked monomer chains are particularly useful for some applications. Polymers useable in the methods, devices and components disclosed include, but are not limited to, plastics, elastomers, thermoplastic elastomers, elastoplastics, thermoplastics and acrylates. Exemplary polymers include, but are not limited to, acetal polymers, biodegradable polymers, cellulosic polymers, fluoropolymers, nylons, polyacrylonitrile polymers, polyamide-imide polymers, polyimides, polyarylates, polybenzimidazole, polybutylene, polycarbonate, polyesters, polyetherimide, polyethylene, polyethylene copolymers and modified polyethylenes, polyketones, poly(methyl methacrylate), polymethylpentene, polyphenylene oxides and polyphenylene sulfides, polyphthalamide, polypropylene, polyurethanes, styrenic resins, sulfone-based resins, vinyl-based resins, rubber (including natural rubber, styrene-butadiene, polybutadiene, neoprene, ethylene-propylene, butyl, nitrile, silicones), acrylic, nylon, polycarbonate, polyester, polyethylene, polypropylene, polystyrene, polyvinyl chloride, polyolefin or any combinations of these.

“Elastomer” refers to a polymeric material which can be stretched or deformed and returned to its original shape without substantial permanent deformation. Elastomers commonly undergo substantially elastic deformations. Useful elastomers include those comprising polymers, copolymers, composite materials or mixtures of polymers and copolymers. Elastomeric layer refers to a layer comprising at least one elastomer. Elastomeric layers may also include dopants and other non-elastomeric materials. Useful elastomers include, but are not limited to, thermoplastic elastomers, styrenic materials, olefinic materials, polyolefin, polyurethane thermoplastic elastomers, polyamides, synthetic rubbers, PDMS, polybutadiene, polyisobutylene, poly(styrene-butadiene-styrene), polyurethanes, polychloroprene and silicones. Exemplary elastomers include, but are not limited to silicon containing polymers such as polysiloxanes including poly(dimethyl siloxane) (i.e. PDMS and h-PDMS), poly(methyl siloxane), partially alkylated poly(methyl siloxane), poly(alkyl methyl siloxane) and poly(phenyl methyl siloxane), silicon modified elastomers, thermoplastic elastomers, styrenic materials, olefinic materials, polyolefin, polyurethane thermoplastic elastomers, polyamides, synthetic rubbers, polyisobutylene, poly(styrene-butadiene-styrene), polyurethanes, polychloroprene and silicones. In an embodiment, a polymer is an elastomer.

“Conformable” refers to a device, material or substrate which has a bending stiffness that is sufficiently low to allow the device, material or substrate to adopt any desired contour profile, for example a contour profile allowing for conformal contact with a surface having a pattern of relief features. In certain embodiments, a desired contour profile is that of skin.

“Conformal contact” refers to contact established between a device and a receiving surface, specifically skin. In one aspect, conformal contact involves a macroscopic adaptation of one or more surfaces (e.g., contact surfaces) of a device to the overall shape of a surface. In another aspect, conformal contact involves a microscopic adaptation of one or more surfaces (e.g., contact surfaces) of a device to a surface resulting in an intimate contact substantially free of voids. In an embodiment, conformal contact involves adaptation of a contact surface(s) of the device to a receiving surface(s) such that intimate contact is achieved, for example, wherein less than 20% of the surface area of a contact surface of the device does not physically contact the receiving surface, or optionally less than 10% of a contact surface of the device does not physically contact the receiving surface, or optionally less than 5% of a contact surface of the device does not physically contact the receiving surface. Devices of certain aspects are capable of establishing conformal contact with internal and external tissue. Devices of certain aspects are capable of establishing conformal contact with tissue surfaces characterized by a range of surface morphologies including planar, curved, contoured, macro-featured and micro-featured surfaces and any combination of these. Devices of certain aspects are capable of establishing conformal contact with tissue surfaces corresponding to tissue undergoing movement.

“Young's modulus” is a mechanical property of a material, device or layer which refers to the ratio of stress to strain for a given substance. Young's modulus may be provided by the expression:

$$E = \frac{( stre s s )}{( strain )} = ( \frac{{L}_{0}}{Δ L} ) ( \frac{F}{A} ) , \qquad ( I )$$

where E is Young's modulus, L₀ is the equilibrium length, ΔL is the length change under the applied stress, F is the force applied, and A is the area over which the force is applied. Young's modulus may also be expressed in terms of Lame constants via the equation:

$$E = \frac{μ ( 3 λ + 2 μ )}{λ + μ} , \qquad ( II )$$

where λ and μ are Lame constants.

“Low modulus” refers to materials having a Young's modulus less than or equal to 10 MPa, less than or equal to 5 MPa or less than or equal to 1 MPa.

“Bending stiffness” is a mechanical property of a material, device or layer describing the resistance of the material, device or layer to an applied bending moment. Generally, bending stiffness is defined as the product of the modulus and area moment of inertia of the material, device or layer. A material having an inhomogeneous bending stiffness may optionally be described in terms of a “bulk” or “average” bending stiffness for the entire layer of material.

“Thermal actuation state” refers to the thermal actuator that is on an off-state or an on-state. In this context, “substantially independent” refers to a position of the reference sensor that is sufficiently separated from the actuator that the reference sensor output is independent of whether the thermal actuator is on or off. Of course, the systems and methods presented herein are compatible with relatively minor effects of the actuator on the reference sensor, such as within 5%, within 1% or within 0.1% of a reference temperature when the actuator is in the on state compared to when the actuator is in the off state. Depending on specific device and tissue characteristics, this distance may be between about 10 mm and 20 mm, such as about 15 mm.

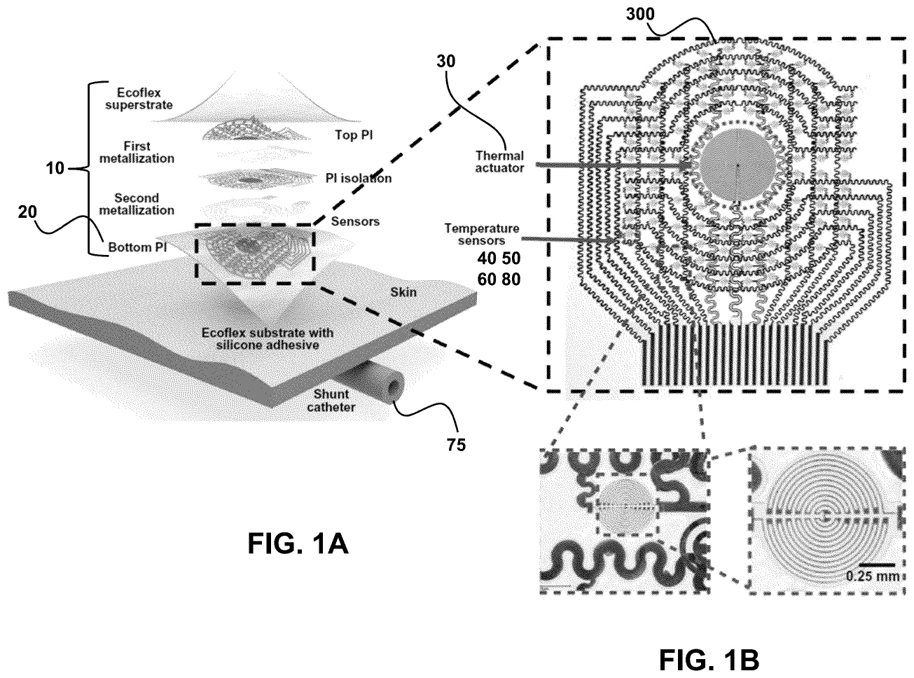

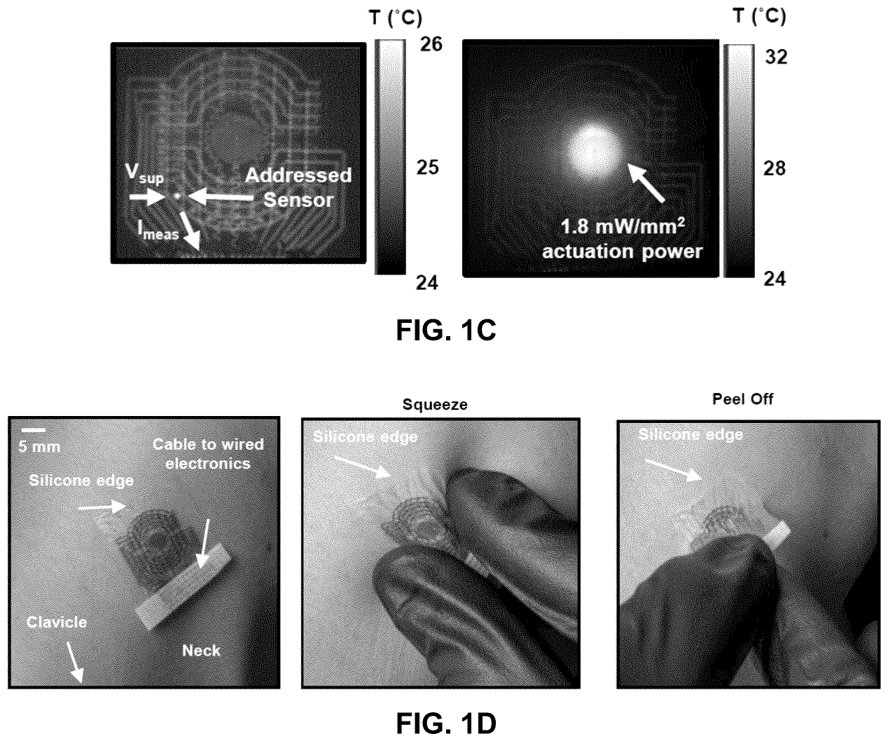

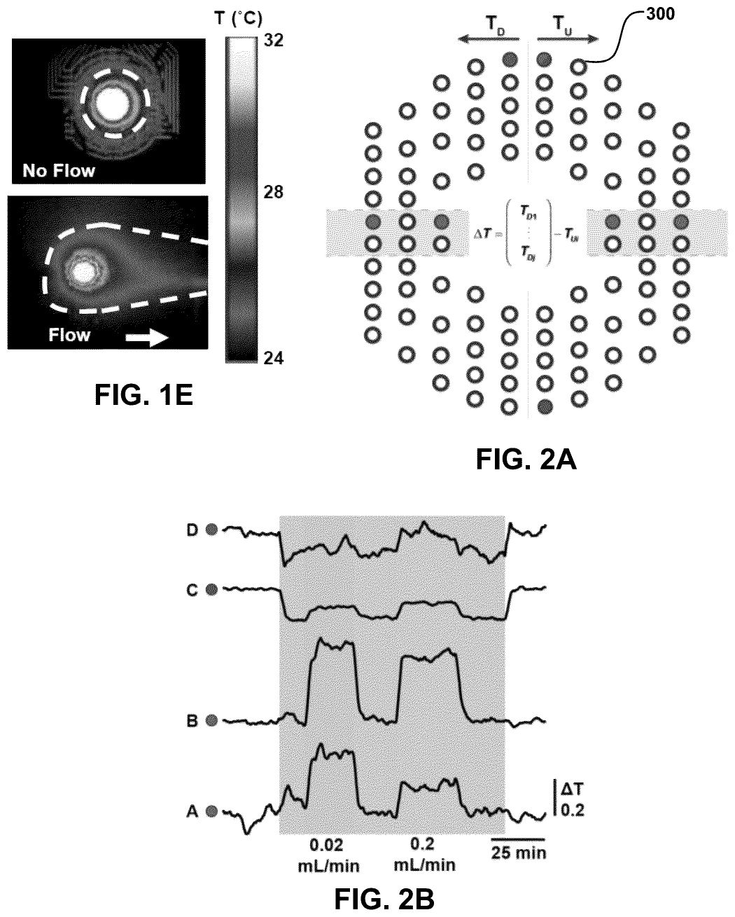

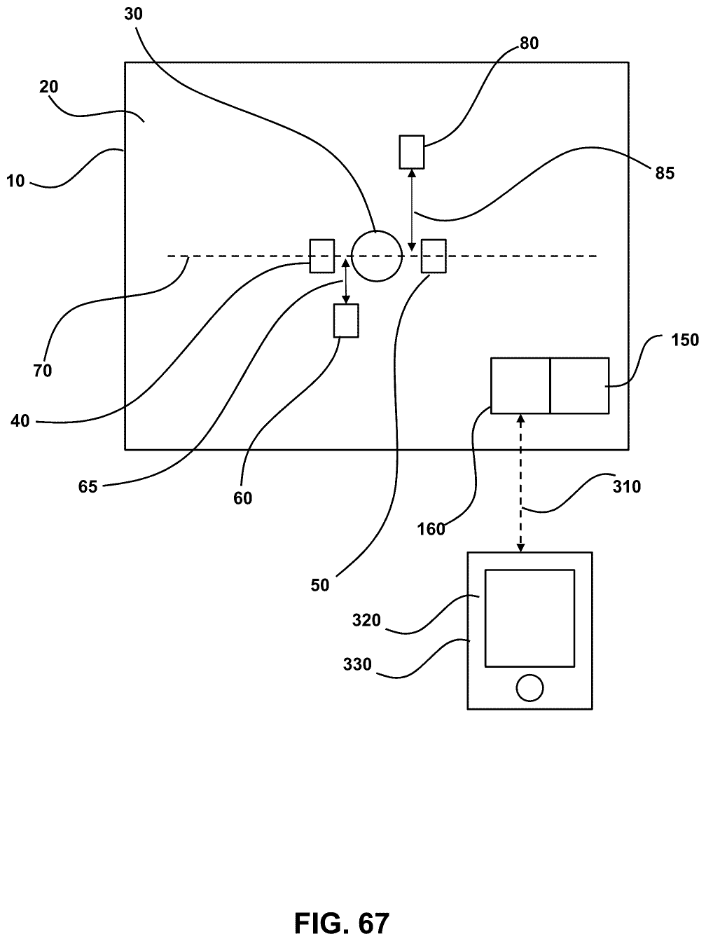

Referring to the figures provided herein, a conformable device 10 to measure subdermal fluid flow, including in a conduit 75 such as a shunt or a blood vessel, may comprise a substrate 20 that supports upstream 30 and downstream 40 temperature sensors. Upstream and downstream are described relative to flow direction in the fluid conduit. The temperature sensors may be part of an array of temperature sensors, including a high density array 300 as shown in FIG. 1A-1B. Within that array, are any number of reference sensors used to assess one or more baseline skin properties, including an actuator reference sensor 60 and/or ambient reference sensor 80. As explained herein, the reference sensor locations may be determined to be those that are independent of thermal actuation status (e.g., ambient reference sensor) or of flow status in the conduit (e.g., actuator reference sensor). The reference sensor locations may be characterized in terms of a separation distance (65 85) from notional line 70 that is a straight line connection between the upstream and downstream temperature sensors and the thermal actuator to the reference sensors (60 80) (FIG. 67).

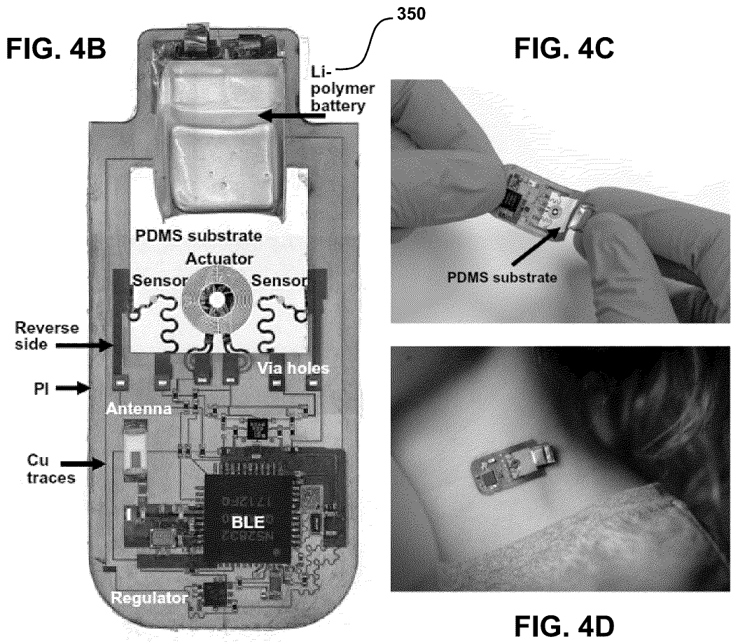

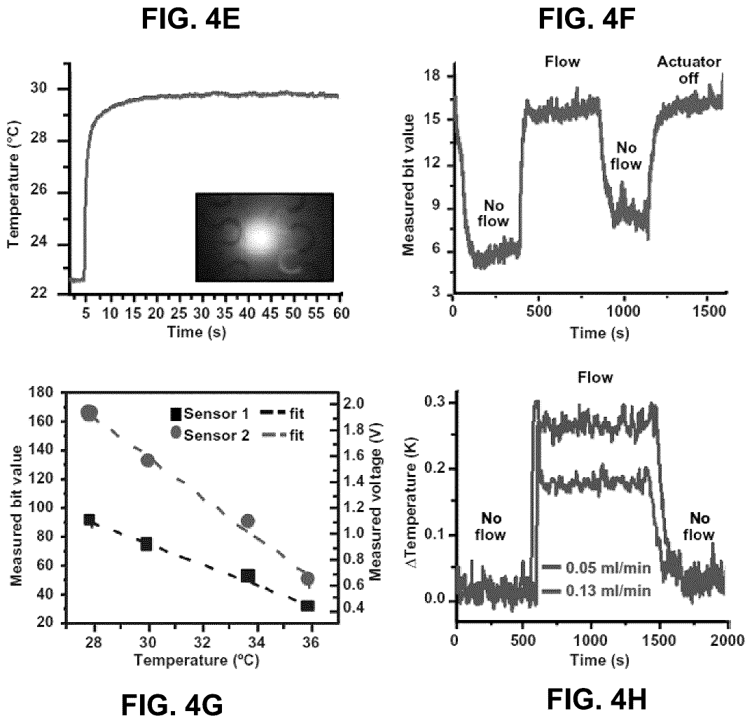

A microprocessor 160 (illustrated as on-board device 10 in FIG. 67, but may be positioned remotely), may be wirelessly connected via one or more wireless communication components 310 to the temperature sensors and/or to connect the device 10 to a microcontroller 320 illustrated as within a hand-held or computer 330 remote device. As desired, a power source 150 (also illustrated as 350 in FIG. 4B) may be connected on-board device 10. Optionally, the power source may correspond to wirelessly charging components. Wireless communication components 5810 are also illustrated in FIG. 58 (see also antenna of FIG. 4B).

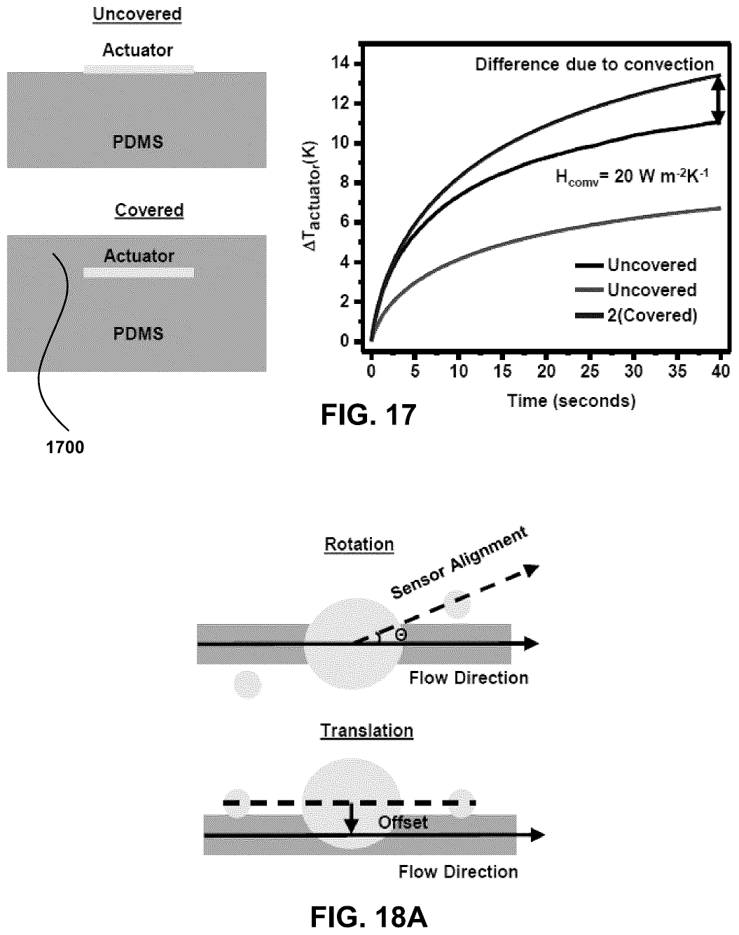

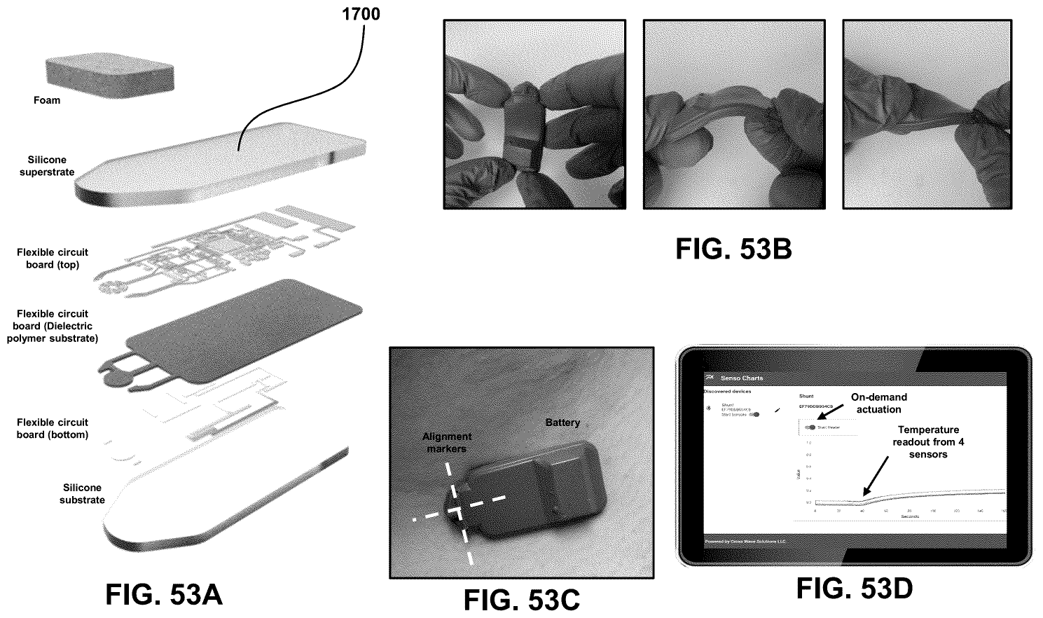

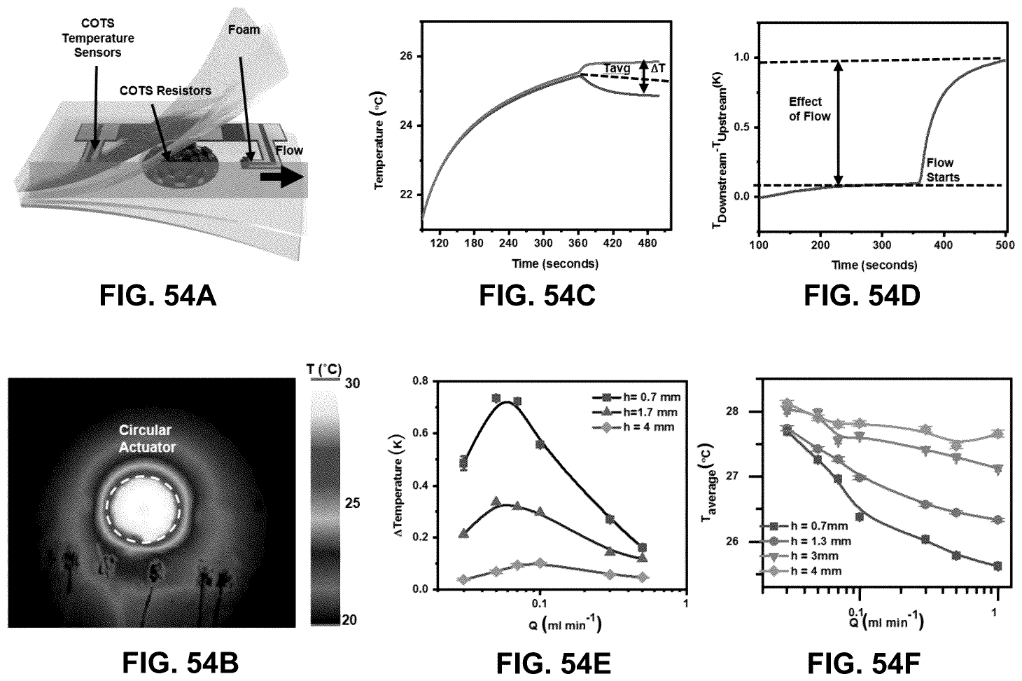

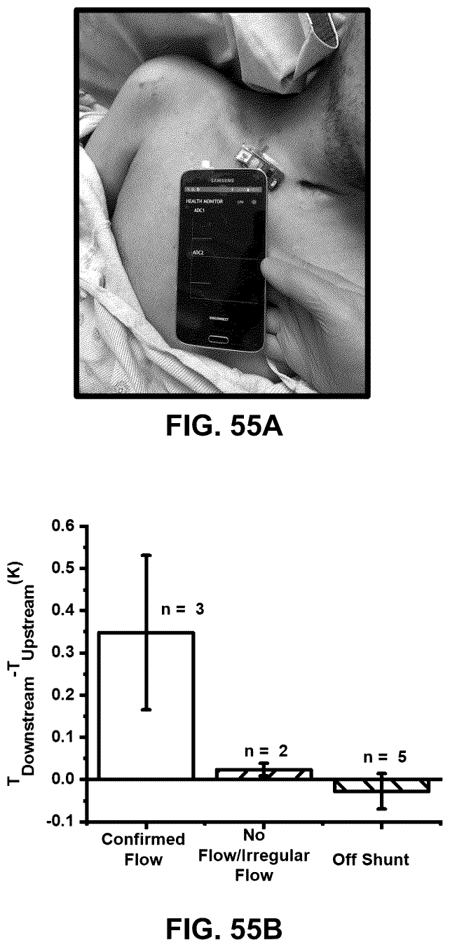

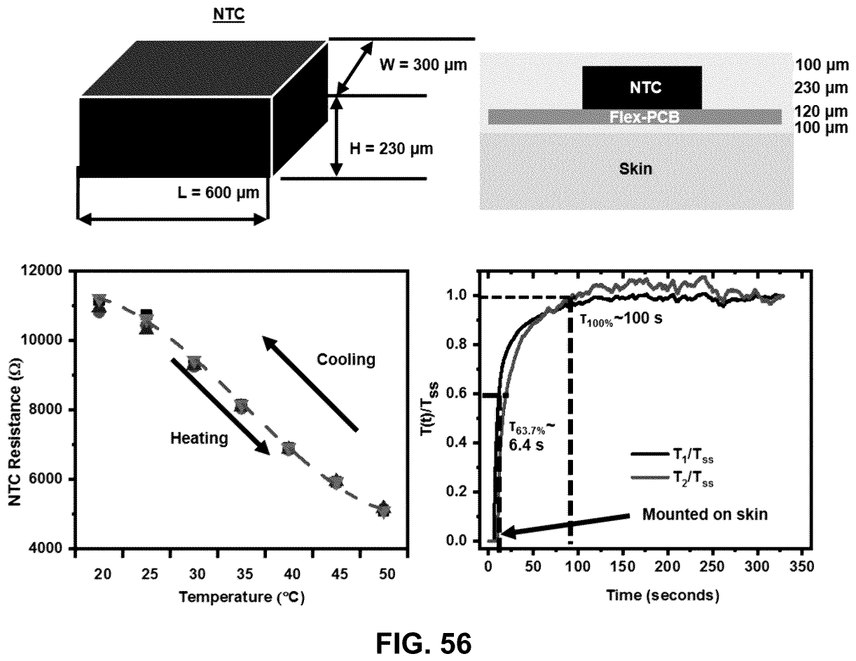

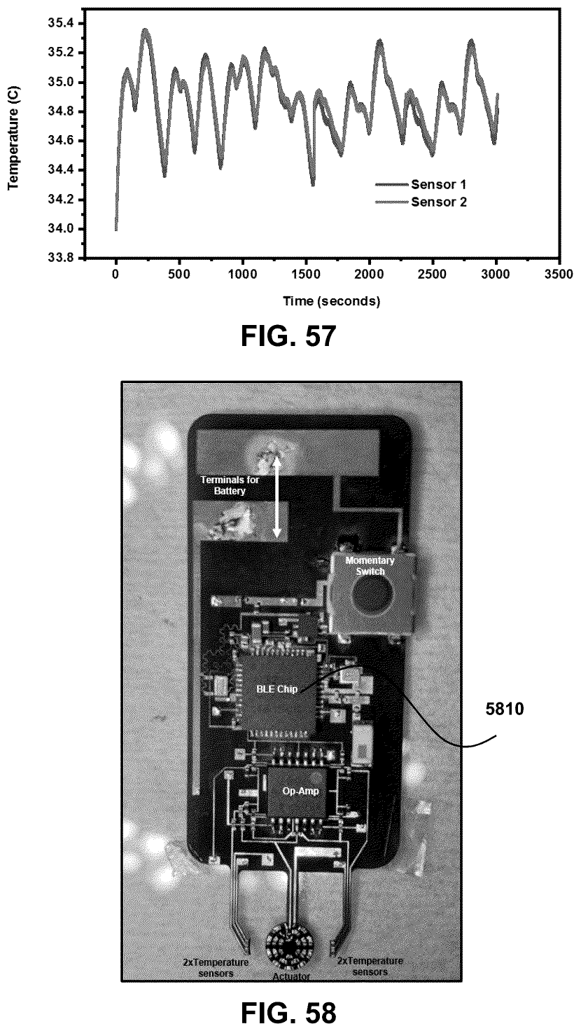

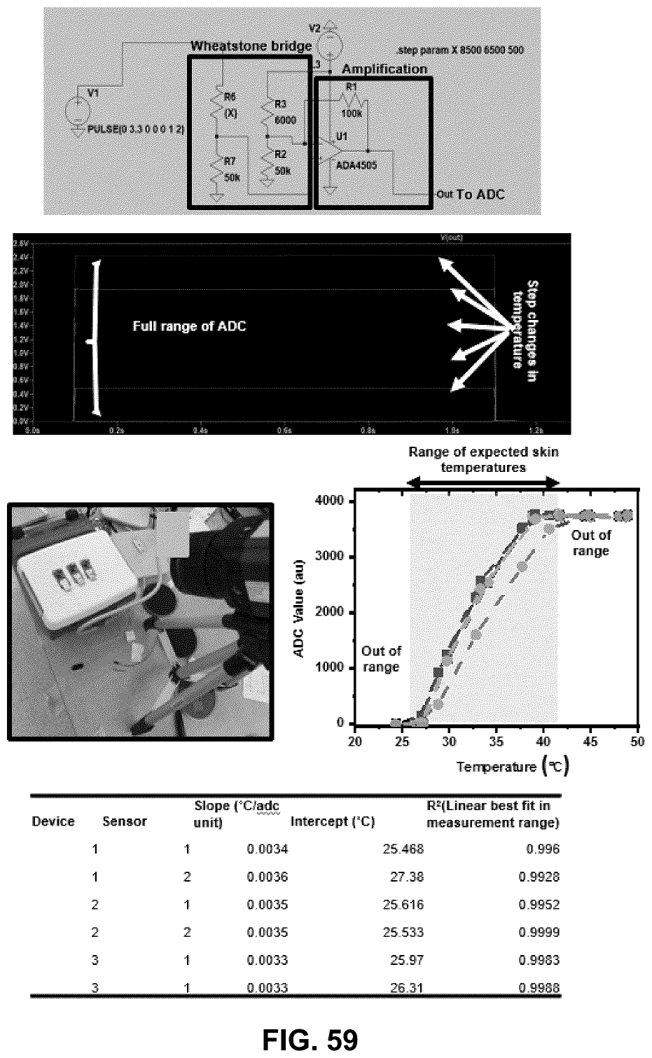

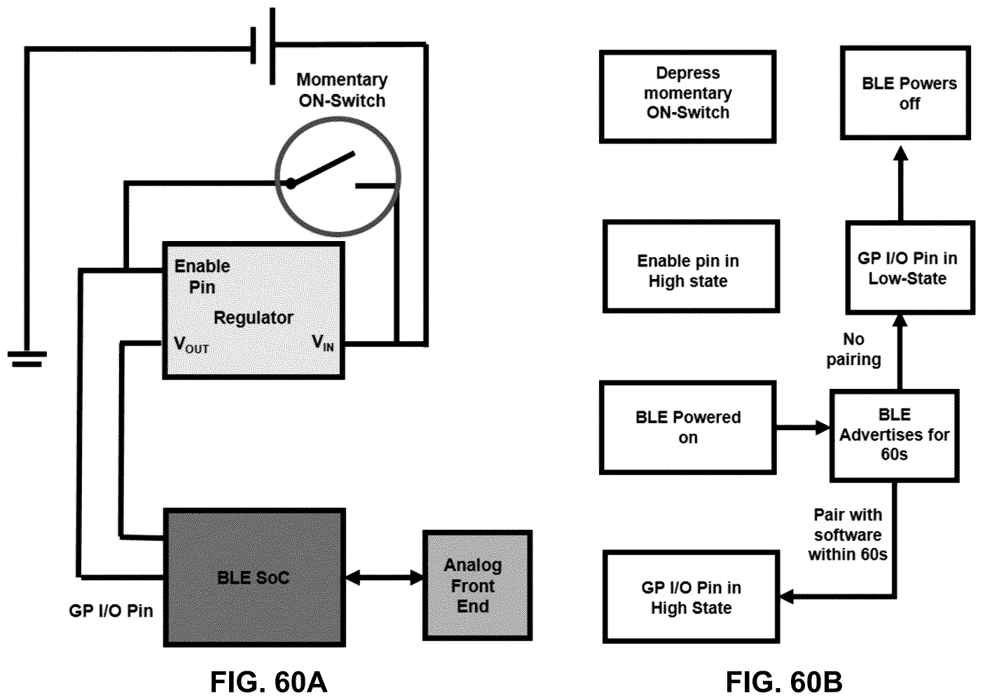

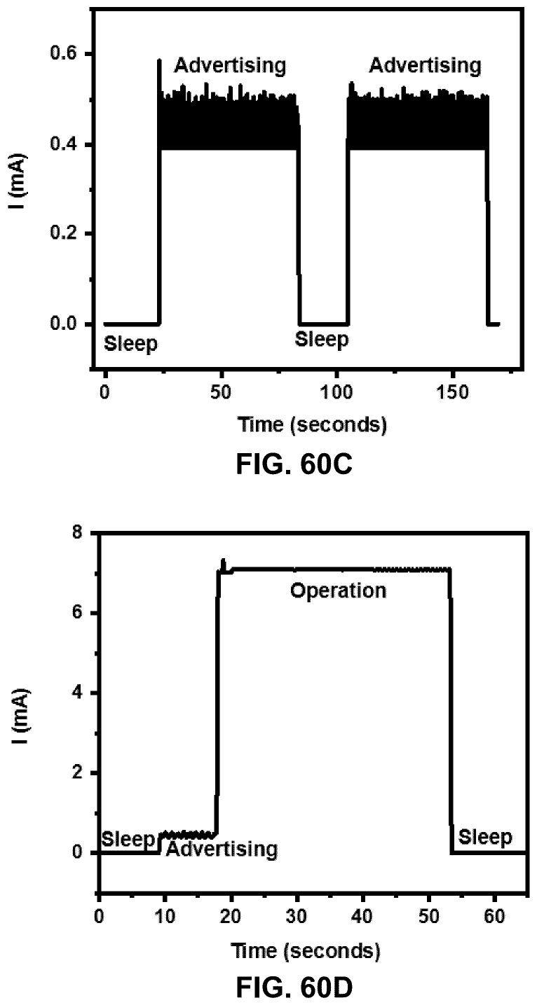

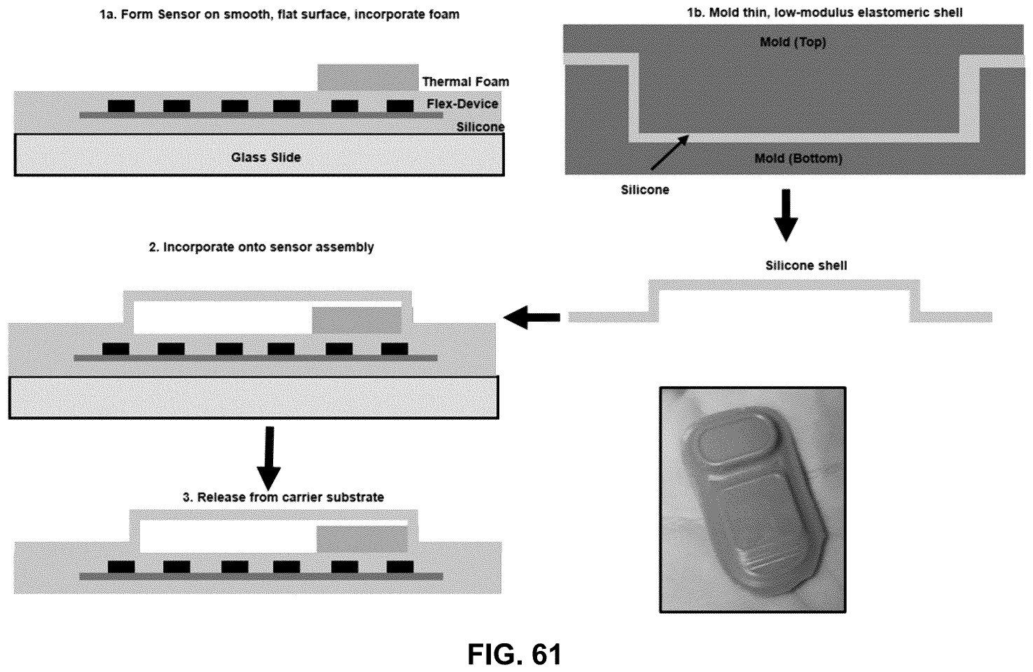

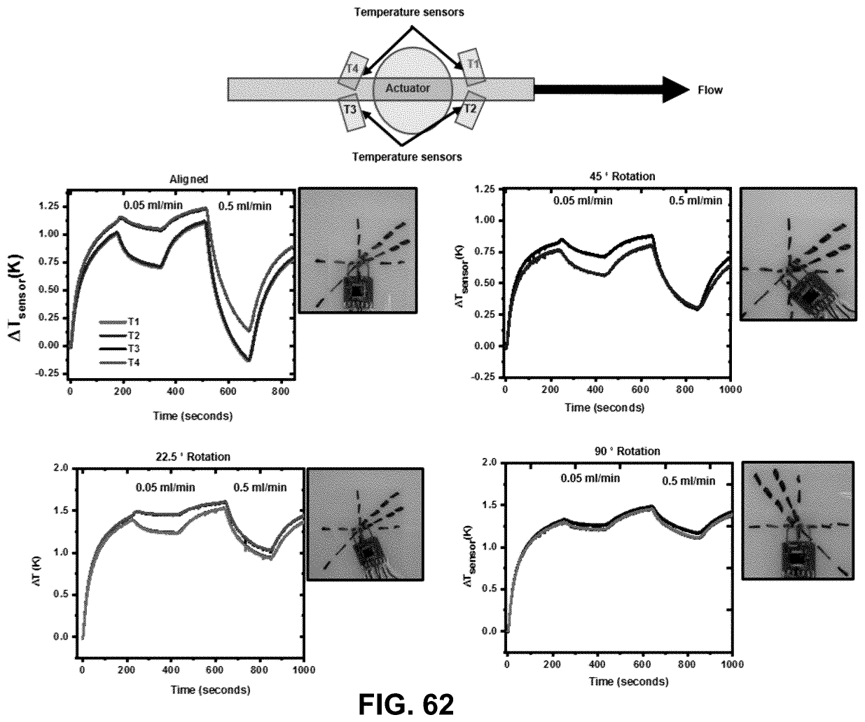

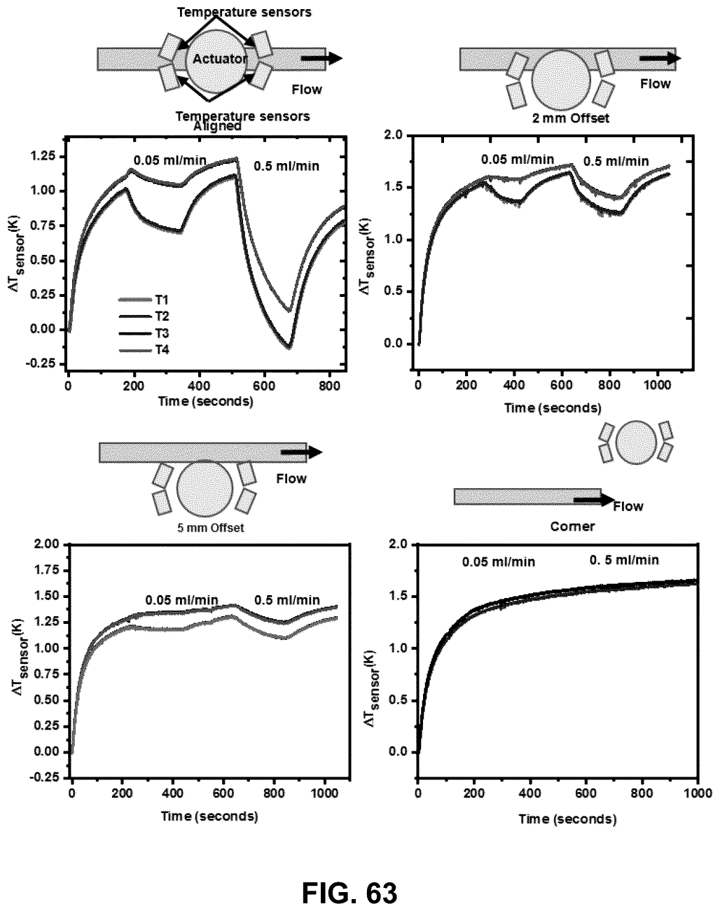

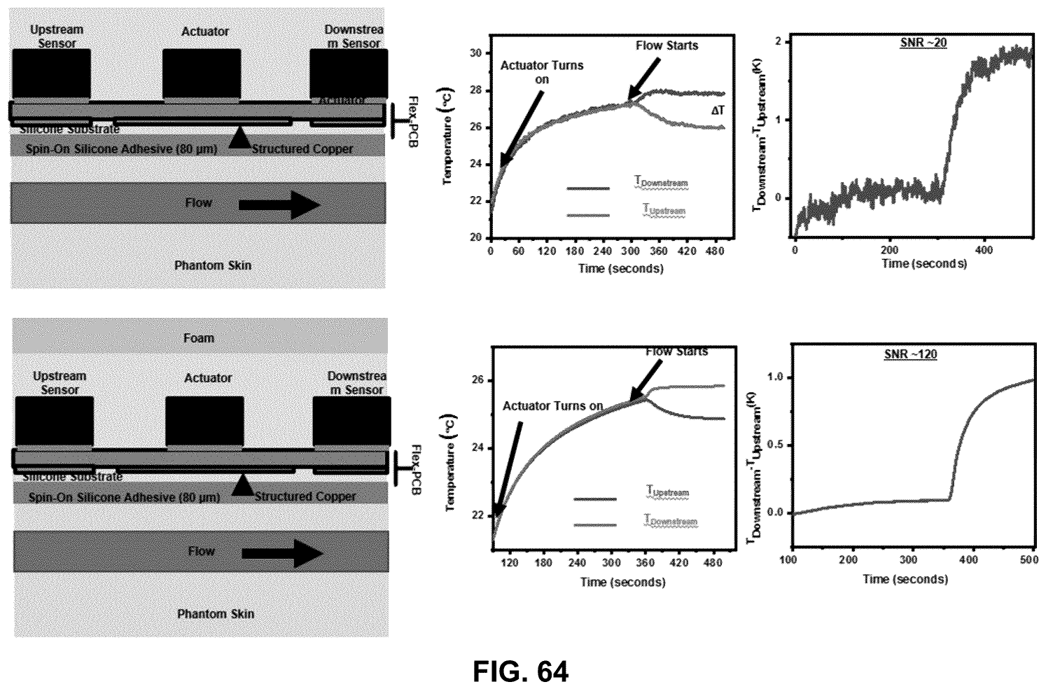

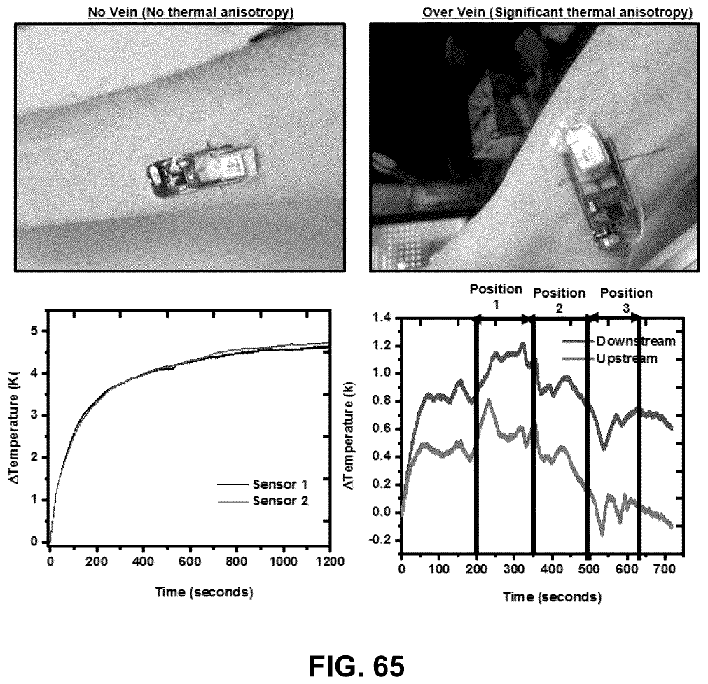

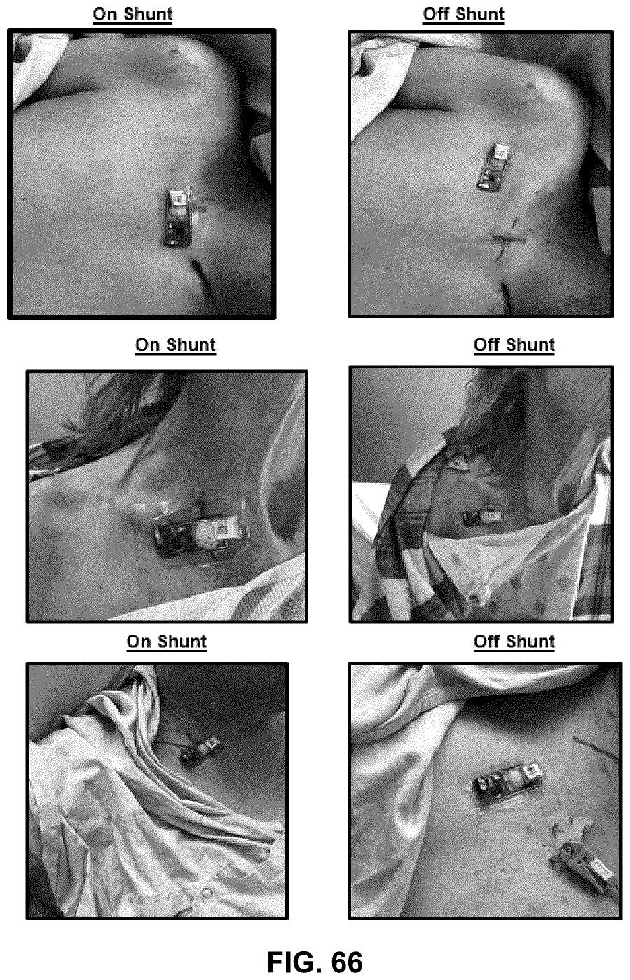

As desired, the device may be covered with an encapsulation layer 1700 (FIG. 17), including a foam layer or an additional partial layer formed of foam on top of the encapsulating layer (also referred herein as a superstrate—see, e.g., FIG. 53A) positioned to vertically cover the temperature actuator and sensors. As the foam layer is demonstrated to improve device performance, any of the devices provided herein may comprise a foam layer positioned over an encapsulation layer, wherein the foam layer may cover the entire encapsulation layer or a portion thereof that corresponds, in a vertical geometrical configuration, to the temperature sensors and actuator to minimize thermal noise and improve device performance and sensitivity.

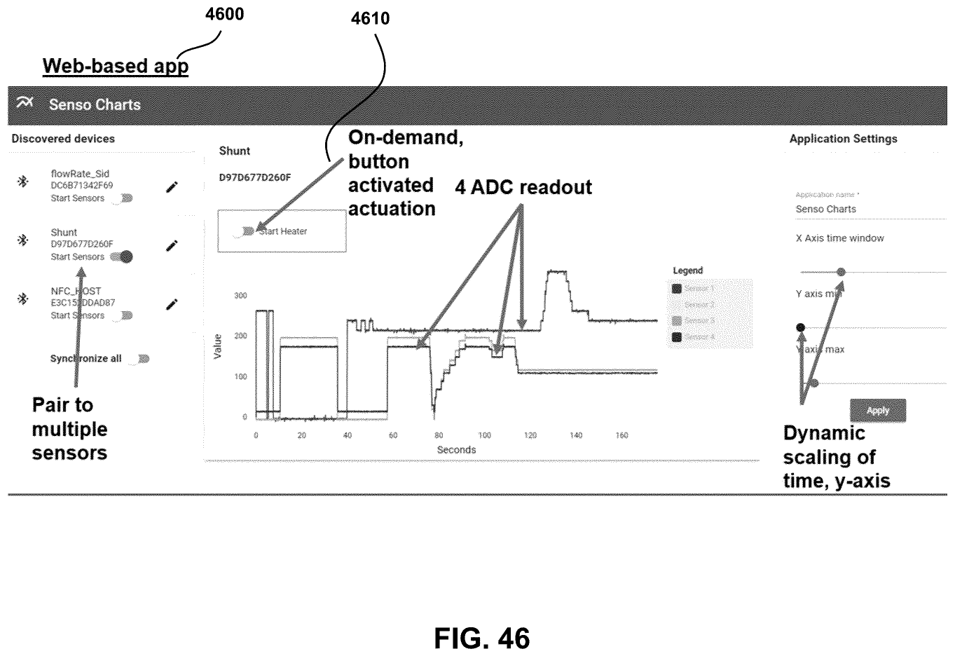

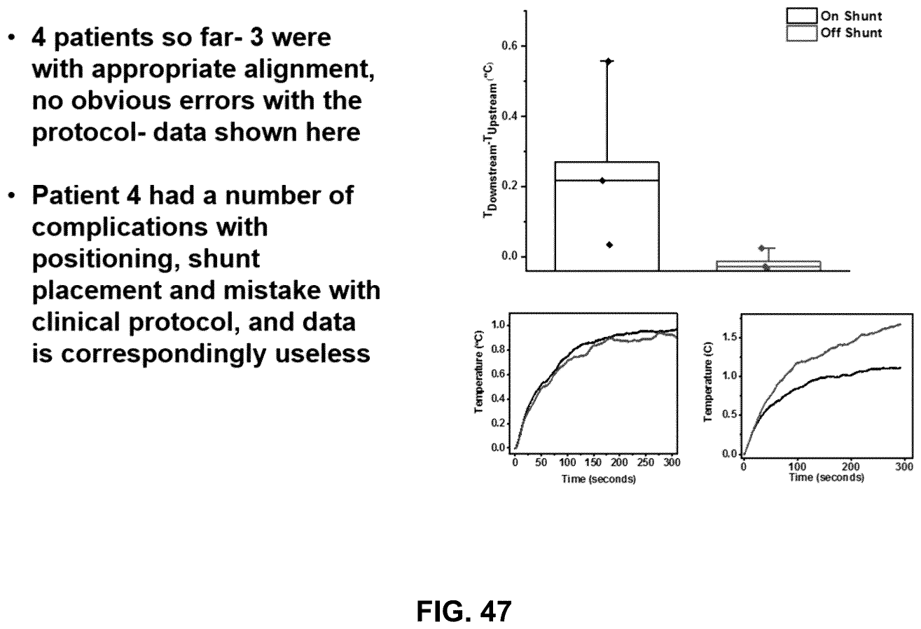

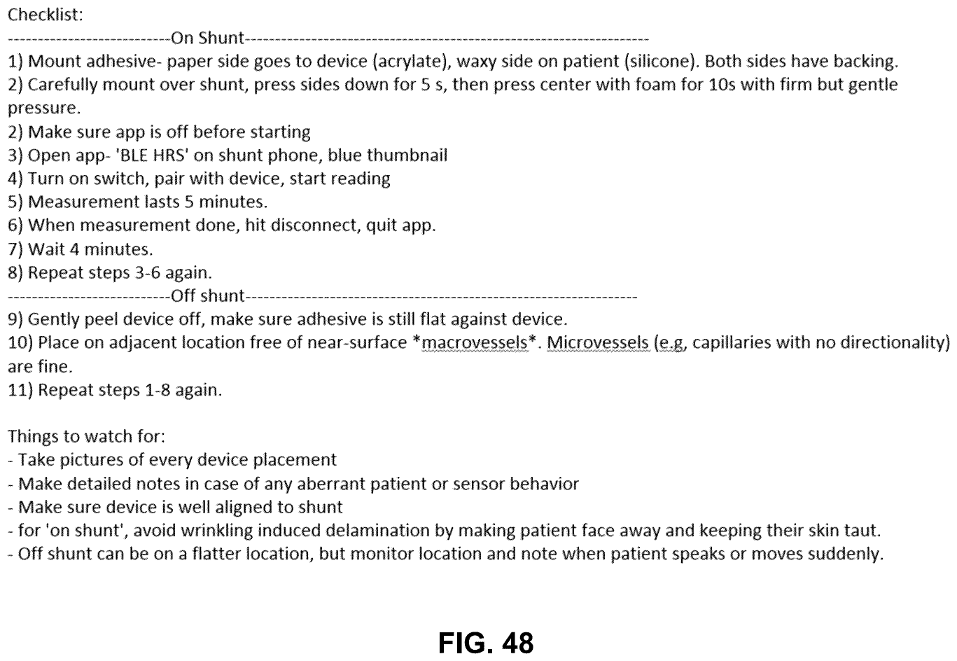

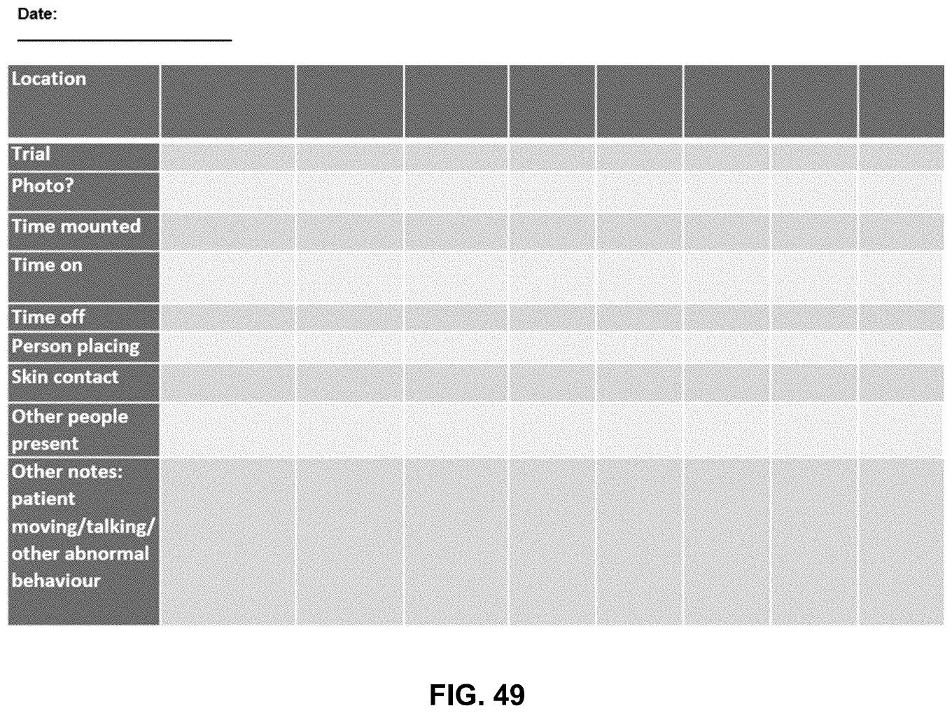

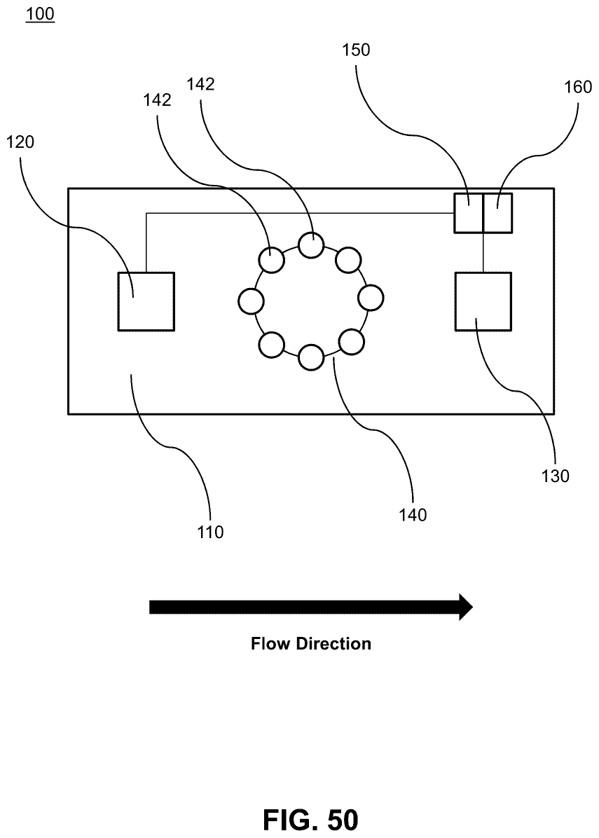

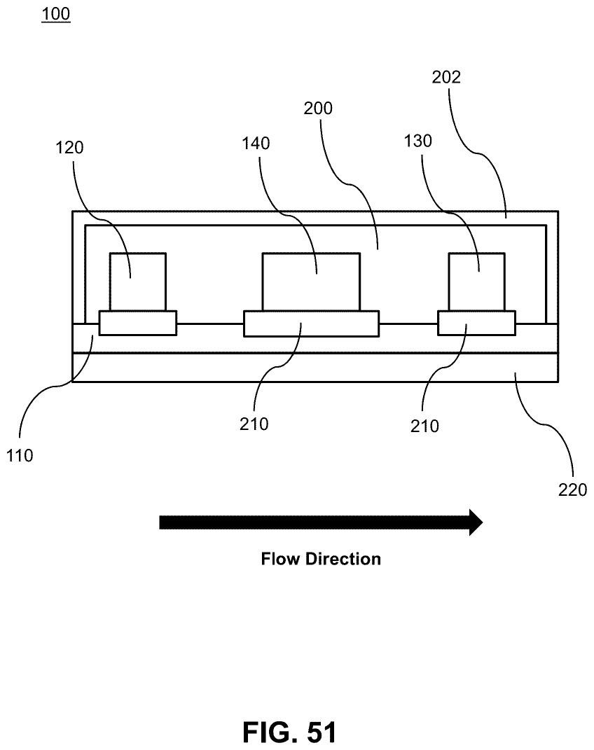

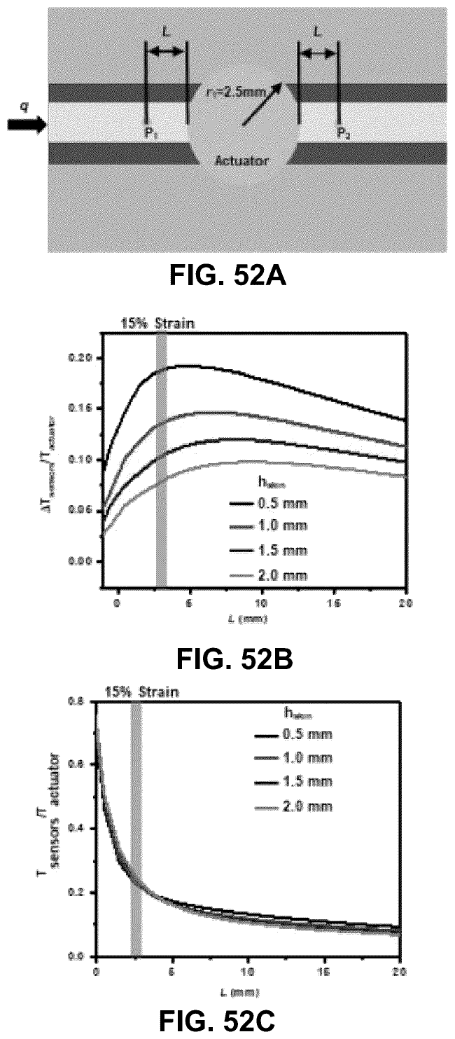

The devices and methods provided herein are conveniently implementable and manageable, including to a health care provider. For example, FIG. 46 illustrates a device operably integrated or connected to a computer-implemented program or application 4600 having an on-demand actuation 4610 on a handheld. In this manner, individual sensor control, device actuation, and date monitoring is readily, conveniently and reliably available to a medical professional who may be remotely located from a patient who is wearing the device.

### Example 1: Epidermal Electronics for the Noninvasive, Wireless, Quantitative Assessment of Ventricular Shunt Function

Ventricular shunts represent an essential component of clinical treatment for hydrocephalus, a common and debilitating neurological disorder that results from the overproduction and/or impaired reabsorption of cerebrospinal fluid (CSF) produced in the ventricular system of the brain [Rachel]. Hydrocephalus arises from a number of causes, including but not limited to cancer, hemorrhage, trauma, and congenital malformations. This condition affects an estimated 750,000 patients in the United States alone, and it is responsible for ˜3.1% of all pediatric acute care costs [Lam, Patwardhan, Shannon, Stone]. 125,000 pediatric hydrocephalus patients in the US account for 400,000 days spent in the hospital each year [Simon]. Shunts assemblies typically involve two silicone catheters, connected upstream and downstream of a regulating valve, to drain excess CSF from the ventricle to a distal absorptive site, usually the peritoneum, pleura, or right atrium of the heart. While effective in CSF diversion and prevention of the sequelae of hydrocephalus, including seizures, coma, neurological injury and death, shunts are highly prone to failure [Tervonen] due to fibrinous catheter ingrowth, kinking, discontinuity, over-drainage, distal malabsorption and infection [Garton, Yuh]. An estimated 84.5% shunt recipients require revision operations [Cochrane, Shah, Stone, Piatt]. Clinical symptoms of shunt malfunction tend to be non-specific, such as headache, nausea and somnolence, thereby creating challenges in clinical diagnosis [Kirkpatrick, Piatt, Garton]. Because ramifications of misdiagnosis can include severe morbidity and mortality, isolating the location and cause of failure is critical in the appropriate care of hydrocephalic patients.

Diagnostic tests to assess shunt function include computerized tomography (CT), plain films (X-Ray), magnetic resonance imaging (MRI), radionuclide shunt patency studies (RSPS, or ‘shunt-o-gram’), shunt aspiration, and flow monitoring systems (ShuntCheck) [Boyle, Wallace, Madsen]. Each method, however, suffers from some combination of disadvantages, including excessive cost, poor reliability, low speeds, susceptibility to interference and patient discomfort, including potential for harm. CT scans and X-rays expose a vulnerable pediatric population to harmful radiation (1.57±0.6 mSv & 1.87±0.45 mSv, respectively). Shunted patients undergo an average of two CT scans annually that, over the course of the patient's lifetime, result in dangerous levels of radiation exposure that have been linked to the onset of neurological and hematological malignancies [Koral, Krishnamurthy]. The MRI approach costs $3000 per study, the measurement can interfere with magnetic shunt valves, the availability is limited, and the wait-times are typically long. Invasive testing, in form of RSPS or simple aspiration, is painful, time-consuming and often inaccurate [Brendel, Ouellette, Uliel, Vernet]. Recent diagnostic entrants attempt to address these drawbacks, but are limited by cumbersome, multi-step protocols, in some cases including ice mediated cooling, with equivocal or negative past clinical data [Madsen, Recinos, Boyle, Frim]. Observation alone can cost over $10,000 per admission, with prolonged hospital stays that compound the frustrations of patients, caregivers, and families alike [Boyle, Yue]. Ultimately, surgical intervention is required to assess and revise shunts in many patients. With risk of intraoperative complications, anesthetic exposure, gross procedural expenditures approach $67,000 per patient [Aqueduct neurosciences. Hydro association]. Because a significant proportion of such surgeries reveal shunt apparatuses with proper flow profiles, these unnecessary procedures represent a tremendous burden to the health care system.

This example presents a simple, non-invasive sensor platform that provides a low-cost, comfortable means for quantitatively assessing flow through cerebrospinal shunts. The platform exploits advances in materials, mechanics and fabrication schemes that serve as the foundations for a class of electronics that is ultrathin (<100 μm), soft (Young's modulus, E˜70 kPa), lightweight (area mass density, <10 mg/cm²) and skin-like it is physical properties, with resulting flexural rigidities that are nine orders of magnitude lower than those of traditional, rigid sensors. Such ‘epidermal’ electronic systems support broad classes of measurement capabilities that offer clinical grade accuracy in capturing body kinematics [1] electrophysiological signals [2,3], soft tissue mechanical properties [4] chemical markers in sweat [5,6] and many others. Multimodal thermal characterization is also possible, owing to the exceptionally low thermal masses (<10 mJ cm⁻²K⁻¹), fast response times (˜10 ms), and exceptional precision in temperature measurements (˜20 mK) of these platforms and to their ability for controlled delivery of thermal power to underlying tissue [7-11]. Specific embodiments allow for high resolution skin thermography and for precise measurements of the thermal conductivity and the thermal diffusivity of the skin. Recent work [12] also illustrates the possibility of quantifying macrovascular blood flow based on measurements of spatial anisotropies in thermal transport. Here, we extend these concepts to realize a soft, skin-interfaced sensor that can accurately measure flow through cerebrospinal shuts in real-time, in a noninvasive, quantitative and wireless manner. The results represent a breakthrough in hydrocephalus diagnostics, with ability to visualize flow in a simple, user-friendly mode, accessible to the physician and patient alike. Systematic benchtop evaluations, thermographic imaging and finite element analysis (FEA) of the physics of heat transport reveal the effects of skin thermal properties and thickness, as well as device and catheter geometries. The results establish considerations in design for a range of practical operating conditions. An integrated wireless system allows for recording and transmission of data to standard consumer devices such as smartphones and tablet computers. Trials on five adult shunt recipients with a diverse range of etiologies, and comparisons with CT, MRI and radionuclide tracing validate device function in-vivo, and advanced processing algorithms for quantitative determination of flow rates.

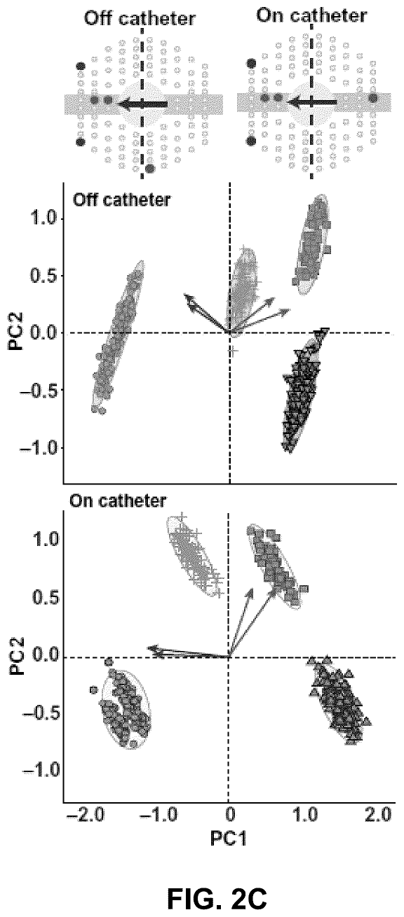

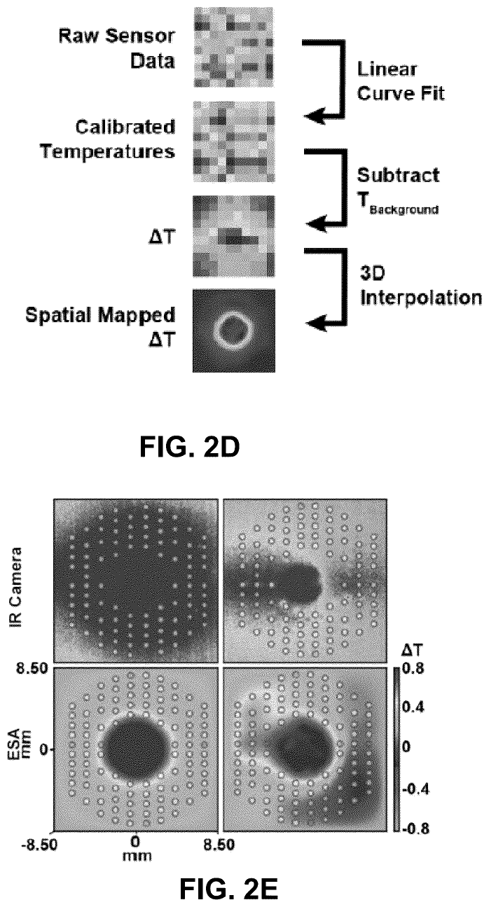

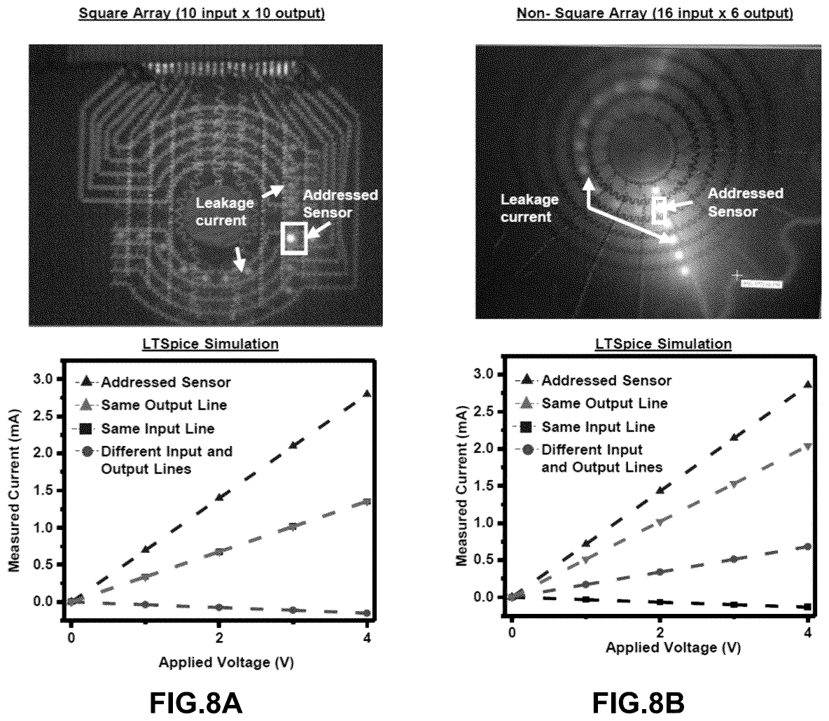

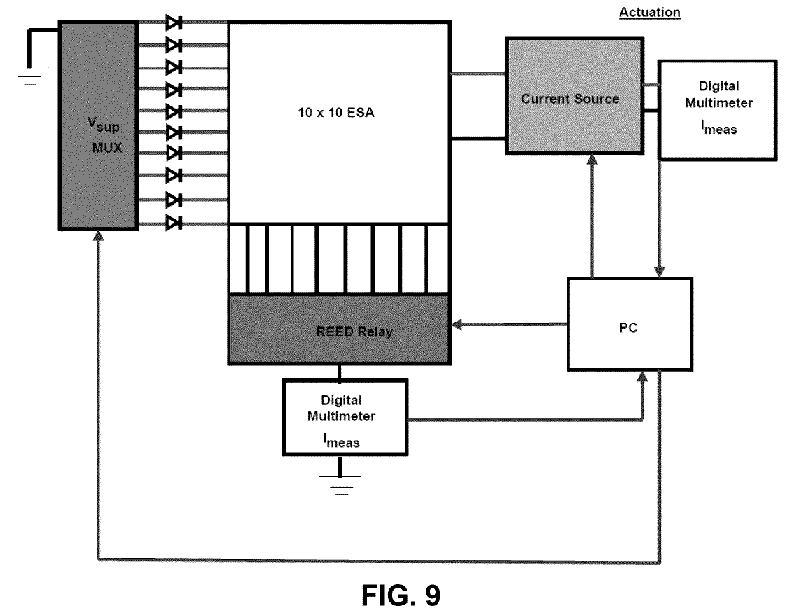

Dense arrays for flow visualization: The feasibility of using arrays of epidermal temperature sensors and thermal actuators to quantify anisotropies in thermal transport through the skin induced by macrovascular blood flow has been demonstrated [10,12]. The device architectures and fabrication schemes shown here increase the number of sensors by nearly a factor of ten relative to this past work, and the density of these elements by a factor of four, using clusters distributed around a central thermal actuator, to provide levels of precision and spatial resolution necessary for characterizing flow through shunts. A schematic illustration of this platform (epidermal sensing array, ESA) appears in FIG. 1A. Optical micrographs of the key features are in FIG. 1B. The device illustrated here comprises a circular (R=2.5 mm) thin-film metallic (Cr/Au 10/50 nm) actuating element surrounded by 100 circular (R=0.25 mm) thin-film metallic (Cr/Au 10/50 nm) temperature sensors. Two layers of metallic traces (Ti/Cu/Ti/Au 20/600/20/25 nm) patterned in serpentine geometries define interconnects between the sensing and actuating elements, with polyimide (PI) as an interlayer dielectric. A film of PI (9 μm total thickness) patterned and aligned to the metal features serves as an encapsulation layer. A soft (70 kPa) substrate, such as an elastomeric substrate (Ecoflex, 100 μm) serves as a support. Connecting unique combinations of rows (to supply a sensing voltage, Vₛᵤₚ) and columns (to measure a resulting current, Iₘₑₐₛ) enables individual addressing of each element in the array, as in FIG. 2C. Operation of the thermal actuator, as seen in the IR thermograph in FIG. 2C, results in a spatio-temporal pattern of temperatures that can be captured by high-speed, automated interrogation of the sensors in the array. An illustration of the data acquisition system appears in FIG. 9. Arrays in square geometries, with an equal number of input and output lines (10×10 for the case illustrated here) mitigate effects of parasitic current pathways. (Theoretical and experimental comparisons of current distributions in square and non-square arrays appear in FIGS. 8A-8B.) The ease of fabrication and robustness of operation of metallic resistive sensor elements make them attractive options compared to semiconductor devices, composite organic thermistors and others. The series of images in FIG. 2E illustrates the mechanical compliance and physical robustness of these systems.

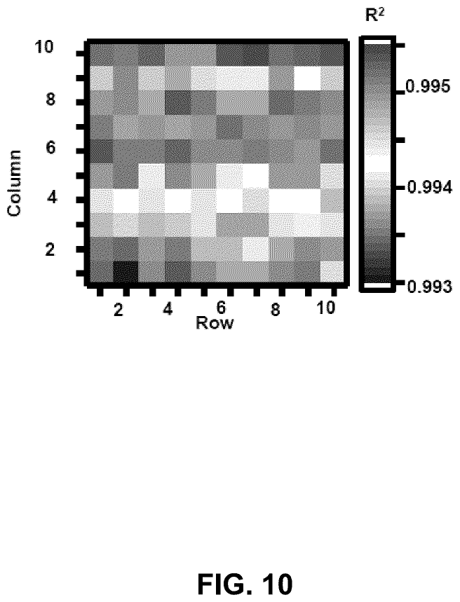

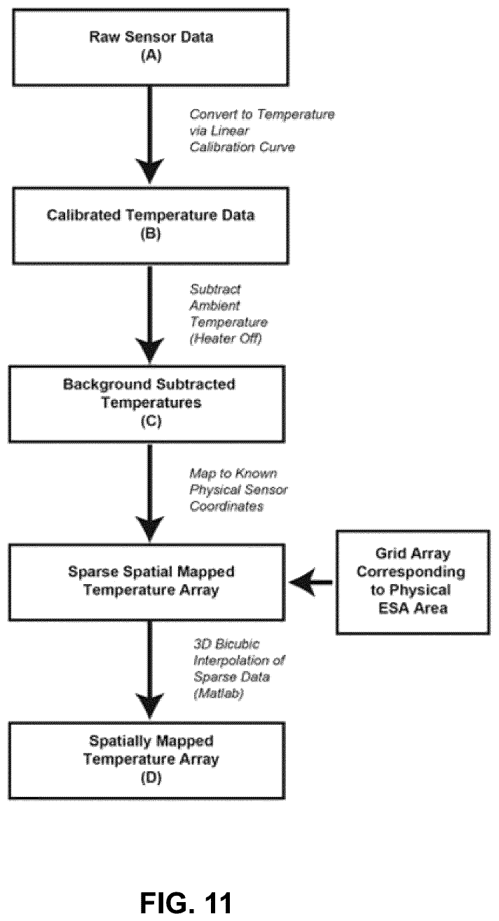

During operation, the current Iₘₑₐₛ that passes through a sensor for a given applied potential defines a resistance that can be converted to temperature via a linear calibration factor, whose goodness of fit is illustrated in FIG. 10. The effects of directional flow through a small diameter tube underlying the device can be seen in the IR thermographs of FIG. 2F. Here, thermal transport occurs most effectively along the direction of flow, thereby creating a pronounced anisotropy in the temperature distribution, with a magnitude that can be quantitatively related to the volumetric flow rate, as discussed subsequently. The layout of the sensing elements allows accurate measurements of this anisotropy for cases relevant to flow through subcutaneous shunts with typical dimensions. By comparison to previously reported platforms for sensing of blood flow, the high-density platforms introduced here (1) obviate the need for perfect alignment with the underlying ventricular shunt, (2) facilitate the use of image processing techniques to visualize flow fields, and (3) allow for statistical approaches to interpreting flow due to the density of information.

The schematic illustration in FIG. 2A identifies a set of 50 sensors upstream (Tᵤₚₛₜᵣₑₐₘ) and downstream (T_{downstream}) of the thermal actuator. Subtracting Tᵤₚₛₜᵣₑₐₘ from T_{downstream} for each physically-matched piecewise sensor pair (indicated by the paired colors in FIG. 2A) yields temperature differentials (ΔTₛₑₙₛₒᵣ) that measure the degree of thermal anisotropy that results from fluid flow. As shown in FIG. 2B, ΔTₛₑₙₛₒᵣₛ for sensor pairs A and B that directly overlay a catheter exhibit strong thermal anisotropy under two different flow conditions (0.02 mL min⁻¹, 0.2 mL min⁻¹) within an established range for CSF flow[13]. Sensor location B displays a higher sensitivity to flow than location A due to the reduced effect of direct thermal conduction from the actuator, relative to anisotropic thermal transport due to fluid flow. Measurements of ΔT for distal sensor pairs orthogonal to the flow direction show weak anisotropy (C) while distal pairs parallel to flow direction (D) show an absence of flow-induced thermal anisotropy. This orientation dependence obviates the requirement for precise sensor alignment to tube direction due to the ESA sensor density and cardinal symmetry.

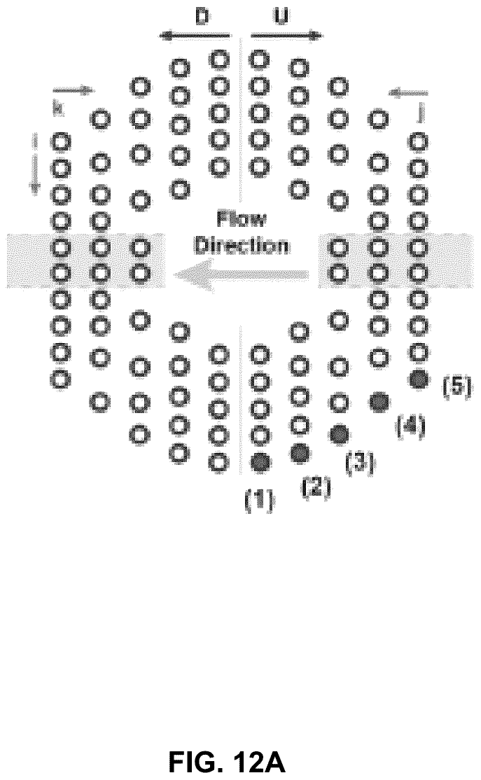

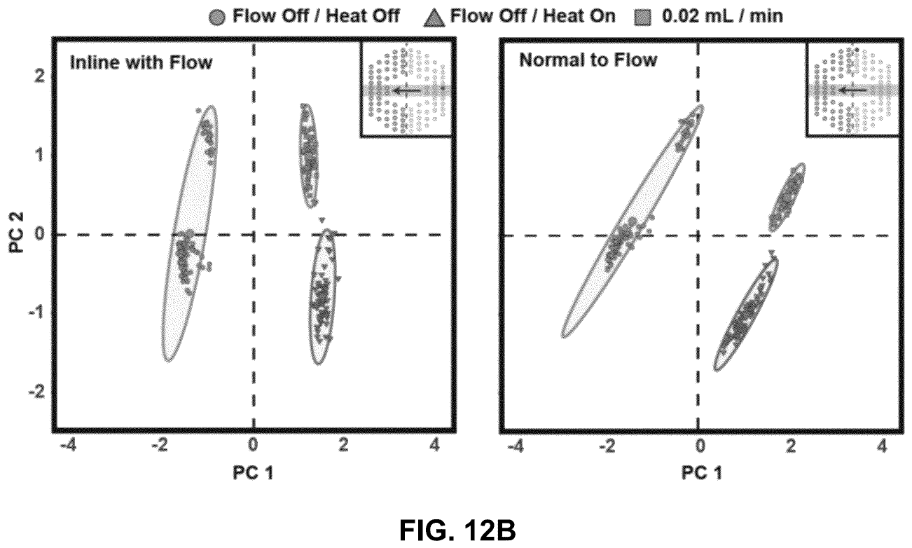

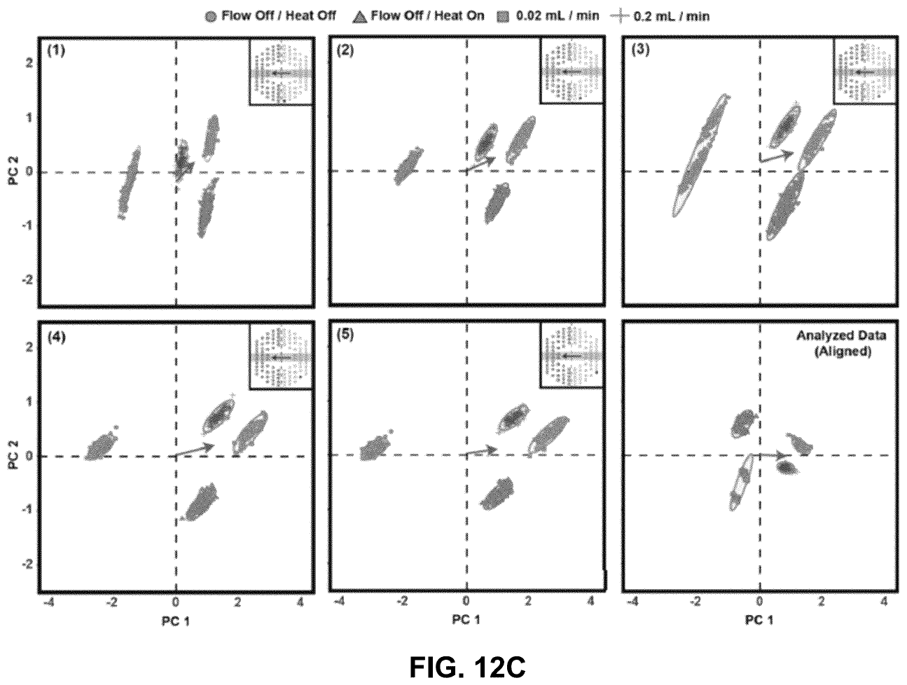

A Principle Component Analysis (PCA) model (generated via R) provides a facile method for assessing both catheter position with respect to the ESA ordinate system and for confirming the presence or absence of flow (shown in FIG. 2C). The PCA model, constructed from a time-series ESA measurement, uses ΔT_{PCA} values to calculate the principle components (PC). ΔT_{PCA}=T_{downstream}−T_{upstream,i}, where T_{downstream} is the temperature matrix of all downstream sensors (1-50) and T_{upstream,i} is the temperature for a single (i) upstream sensor. The first two components (PC1, PC2) describe approximately 92% of the overall variability of the data (70.5% PC1, 22.1% PC2) with the remainder (8% across PC3:PC50) associated with noise. PCA biplots (FIG. 2C) show projections of each ΔT_{PCA} for two selected T_{upstream,i} sensors (Top—orthogonal, distal sensor in red; Bottom—inline, distal sensor in red) at each measurement in an ESA time-series (same as FIG. 2B) in two dimensions using the first two principle components. FIG. 2C reveals data clustering (95% confidence ellipses) corresponding to three experimental conditions: absence of fluid flow without thermal actuation (flow off/heat off), absence of flow with thermal actuation (flow off/heat on), and fluid flow with thermal actuation with separate clusters for different flow regimes (0.02 mL min⁻¹, 0.2 mL min⁻¹). As shown, these clusters are independent of the selected T_{Ui} sensor (additional biplots shown in FIGS. 12A-12B). A comparison of the data clusters and principal components shows that PC1 primarily relates to the degree of thermal actuation while PC2 relates to the presence or absence of flow. Mapping the variables to the PCA biplot indicates sensor correlation to fluid flow. In FIG. 2C, an overlay of four variable factors corresponding to TD sensors known to be proximal (red) and distal (blue) to fluid flow shows the positive correlation for the proximal sensors and negative correlation for the distal sensors to fluid flow for both orthogonal and inline T_{upstream,i} sensors. PCA offers a strategy to mitigate effects of ESA misalignment by determining the T_{upstream, i} sensor that yields the maximal separation between no flow/flow data clusters (along the PC2 axis). As observed in FIG. 2C, the inline T_{upstream, i} sensor strongly separates these cluster groups as compared to the orthogonal T_{upstream,i} sensor. In this manner, for scenarios without a priori orientation, PCA offers a straightforward means for evaluating correlations between T_{upstream, i} and flow state and, therefore, orientation of the catheter relative to the ESA.

The density of the ESA enables spatial mapping of the temperature anisotropy that results from fluid flow. These maps result from the processing of raw measurements from the ESA as outlined in FIG. 2D. First, by converting the raw ESA measurements (Iₘₑₐₛ) to resistance and then temperatures by linear calibration (curve a priori established for each sensor of the ESA, process described in detail in FIG. 9), the temperature values can be mapped to the physical spatial coordinates of each sensor on a simulated square “pixel” array larger than the ESA (grid: 17 mm×17 mm, 10 px mm⁻¹) resulting in a 170×170×N matrix for a time-series measurement of N frames. Conversion to T_{normalized} results from the subtraction of the background temperature T_{background} from each frame. The temperature map results from fitting a surface to the measured T_{normalized} values for each frame via meshed bicubic interpolation (boundary conditions T_{normalized}=0 from IR thermograph). Subtracting the actuator temperature and resulting isotropic heat transfer temperatures (T_{actuator}) from T_{normalized} for every frame enhances visualization of flow-induced anisotropic thermal transport. FIG. 2D compares the ESA temperature maps with IR thermographs (same scale) in the absence (left) and presence (right) of flow (0.02 mL min⁻¹). As seen via the sensor overlay in each image, the high density of the ESA enables good fidelity in visualizing the thermal anisotropy over the embedded catheter. Although experiments with patients do not typically allow for direct measurements of the flow and no-flow cases, theoretically derived or a priori measured “calibration” T_{actuator} facilitates the type of analysis described here.

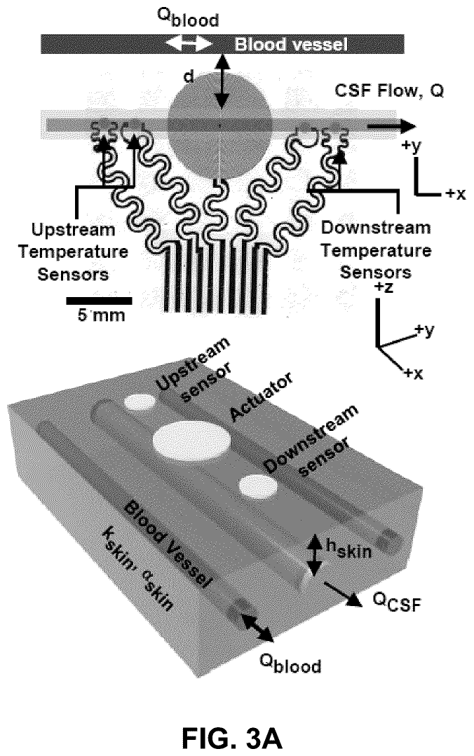

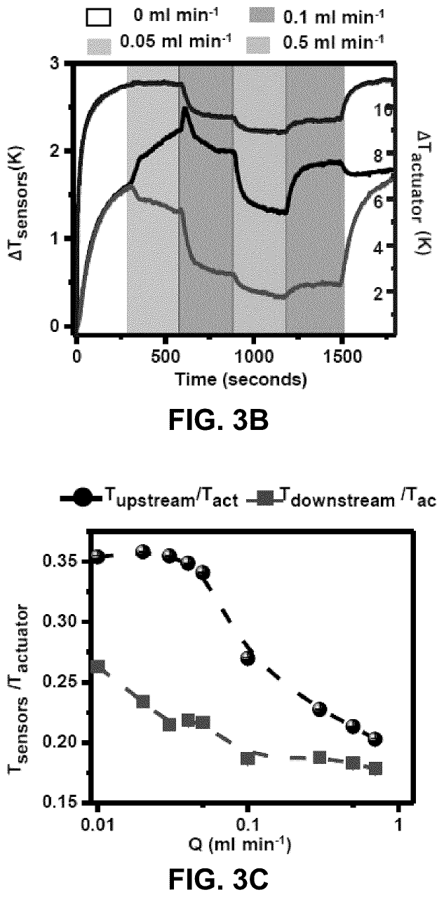

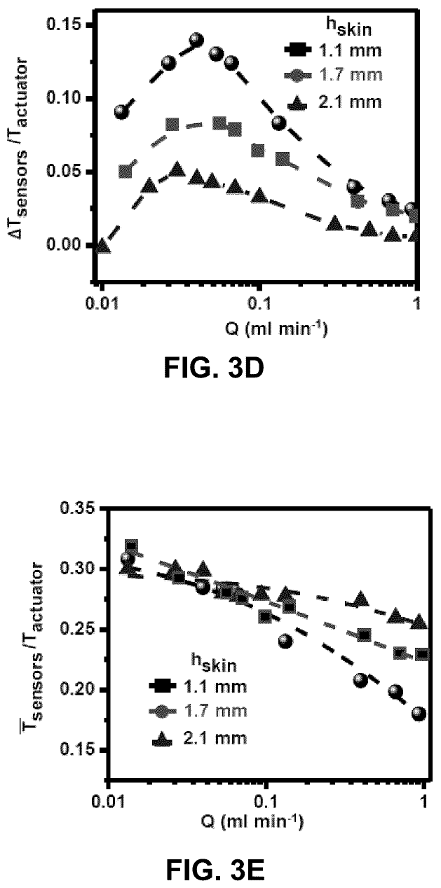

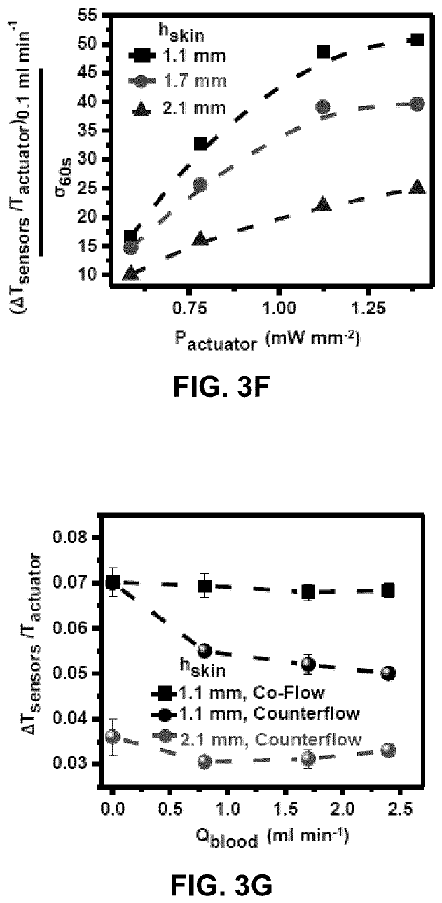

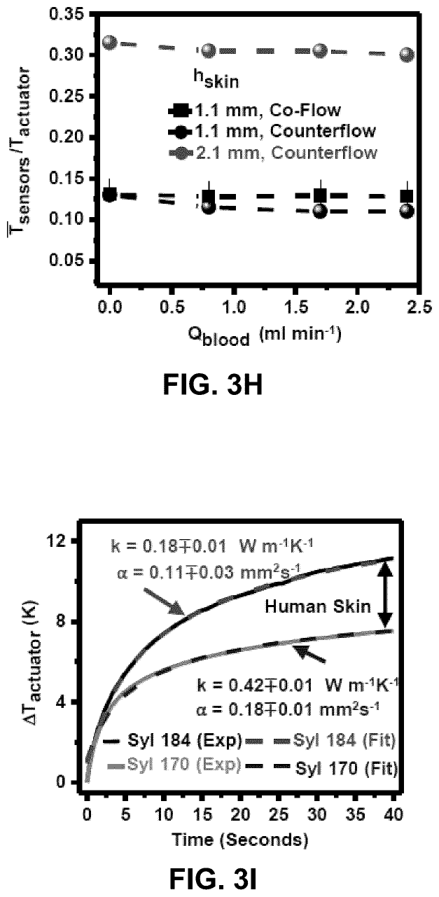

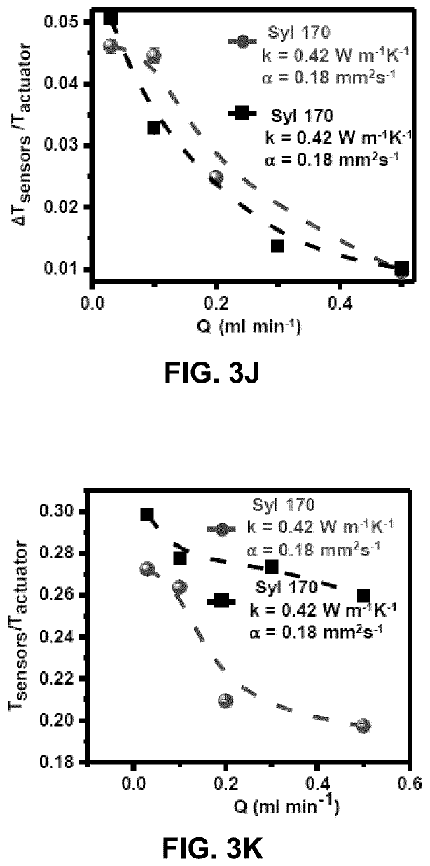

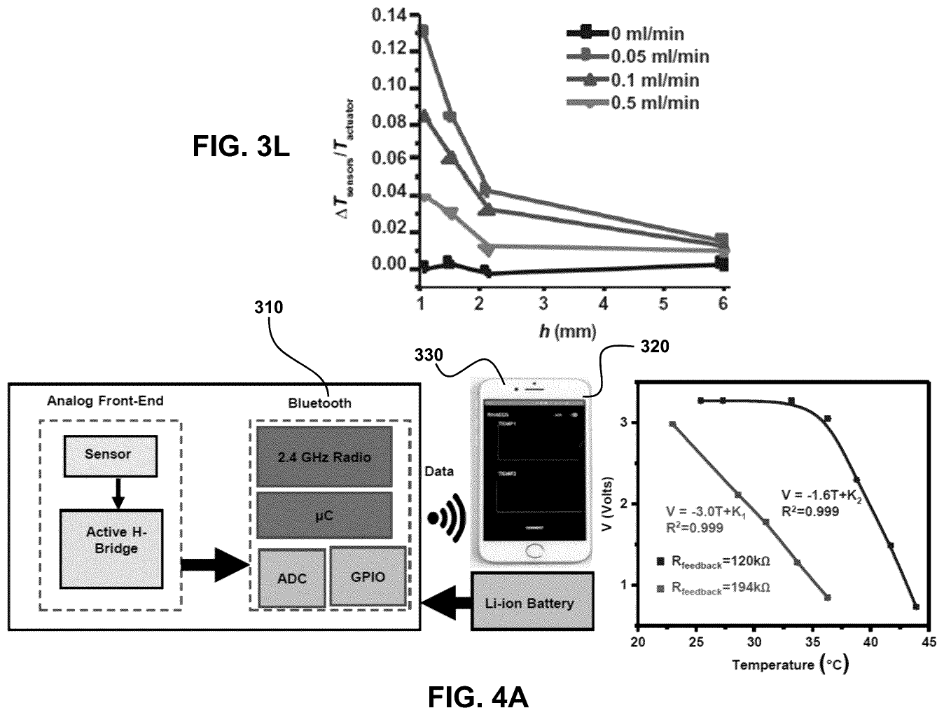

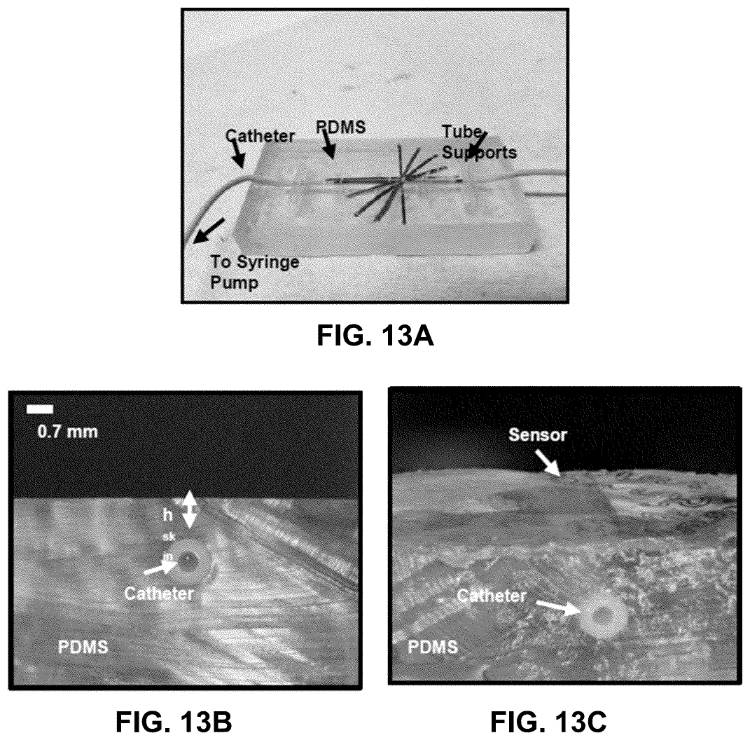

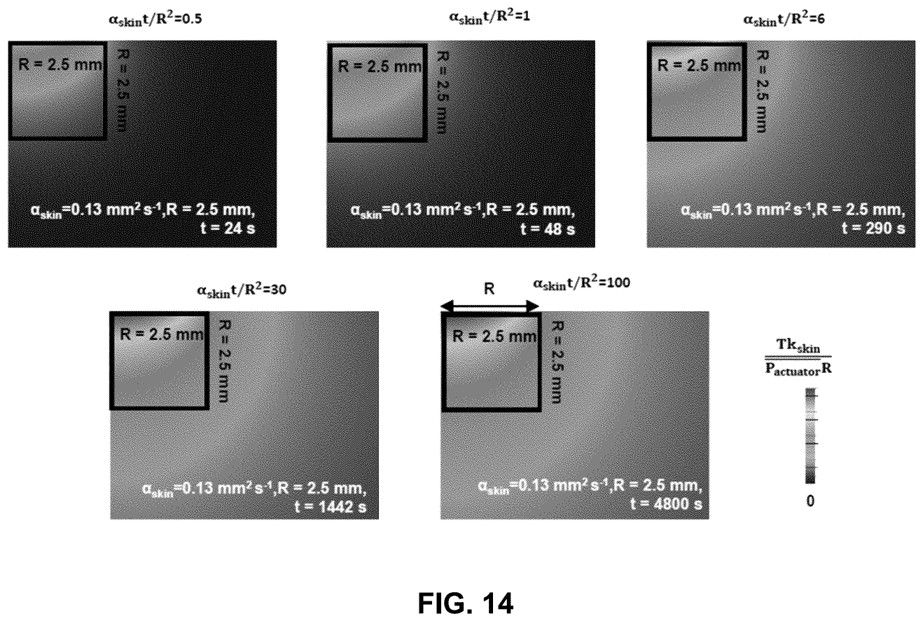

Quantitative analysis of flow and comparison to models: The full mapping results obtained with the high-density ESA suggest means for simplifying the sensor to allow rapid measurements in a low-cost platform that comprises at its core only of an actuator and a pair of sensors, located 1.5 mm upstream (Tᵤₚₛₜᵣₑₐₘ) and downstream (T_{downstream}) of the actuator respectively, which we refer to as an epidermal linear array (ELA). In this system, the actuator simultaneously serves as a temperature sensor, and the measured temperature of the actuator, T_{actuator}, yields a useful normalizing factor that facilitates data analysis independent of actuation power. Use of this system with a benchtop model allows for the controlled exploration of the effects of flow, thermal and geometric parameters. A schematic illustration of the device and evaluation set-up appear in FIG. 3A, with optical images of a representative system appearing in FIGS. 13A-13C. Operating the actuator at a controlled, low-power (1.35 mW/mm²) level creates heat that diffuses through the silicone skin phantom (silicone) at a rate governed by the thermal diffusivity of this material, αₛₖᵢₙ. A scaling law that graphically illustrates the depth of penetration of this thermal field into the phantom appears in FIG. 14. Here, the phantom can be treated as a semi-infinite solid[14], which approaches a quasi-steady state equilibrium over relatively long (˜400 s) times with a corresponding penetration depth of ˜5 mm. Typical ventricular catheters are implanted subdermally, at depths of 1-2 mm [15], well within the range of detectability. The raw transient sensor and actuator responses after actuation (ΔTₛₑₙₛₒᵣₛ=Tₛₑₙₛₒᵣ(t)−Tₛₑₙₛₒᵣ(t_{actuation}), ΔT_{actuator}=T_{actuator}(t)−T_{actuator}(t_{actuation})), and during different flows (Q_{CSF}) in this system appear in FIG. 3B. In the absence of flow (Q_{CSF}=0) thermal transport from the actuator occurs equally in the ∓ee x,∓y and −z directions, resulting in equal values for ΔTᵤₚₛₜᵣₑₐₘ, and ΔT_{downstream}. This regime appears in the unshaded portion of FIG. 3B. The presence of flow leads to a non-monotonic effect on ΔTᵤₚₛₜᵣₑₐₘ, and ΔT_{downstream}. At low flow rates (0 mL min⁻¹<Q_{CSF}<0.05 mL min⁻¹), the fluid serves to transport heat from the actuator preferentially to the downstream sensor, and away from the upstream sensor, resulting in a measured increase in ΔT_{downstream}, and decrease in ΔTᵤₚₛₜᵣₑₐₘ, as seen in the blue shaded region in FIG. 3B. At higher flow rates (0.05 mL min⁻¹<Q_{CSF}<1 mL min⁻¹), the convective effects of the fluid dominate, leading to a net cooling effect on both sensors, but at different rates, with ΔTᵤₚₛₜᵣₑₐₘ equilibrating at a lower value than ΔT_{downstream} as seen in the red and black shaded regions in FIG. 3B. The actuator is convectively cooled by the fluid at a rate governed by the magnitude of the flow, resulting in reductions of ΔT_{actuator}, in the presence of flow as shown by the blue curve in FIG. 3B. These effects appear in the normalized quantities Tᵤₚₛₜᵣₑₐₘ/T_{actuator} and Tᵤₚₛₜᵣₑₐₘ/T_{actuator}, shown for a complete range of physiologically relevant values of Q_{CSF} in FIG. 3C. The non-monotonic effects of flow for different skin thicknesses (hₛₖᵢₙ) increase and decrease when considering the difference between the sensors (ΔTₛₑₙₛₒᵣₛ/T_{actuator}) and their average (Tₛₑₙₛₒᵣₛ/T_{actuator}), respectively, as shown in FIGS. 3D-3E. Here, ΔTₛₑₙₛₒᵣₛ/T_{actuator} and Tₛₑₙₛₒᵣₛ/T_{actuator} are measures of thermal anisotropy and flow magnitude, respectively. Taken together, these quantities allow for determination of flow rate, and can be used to distinguish degenerate points on either side of the peak values shown in FIG. 3D.

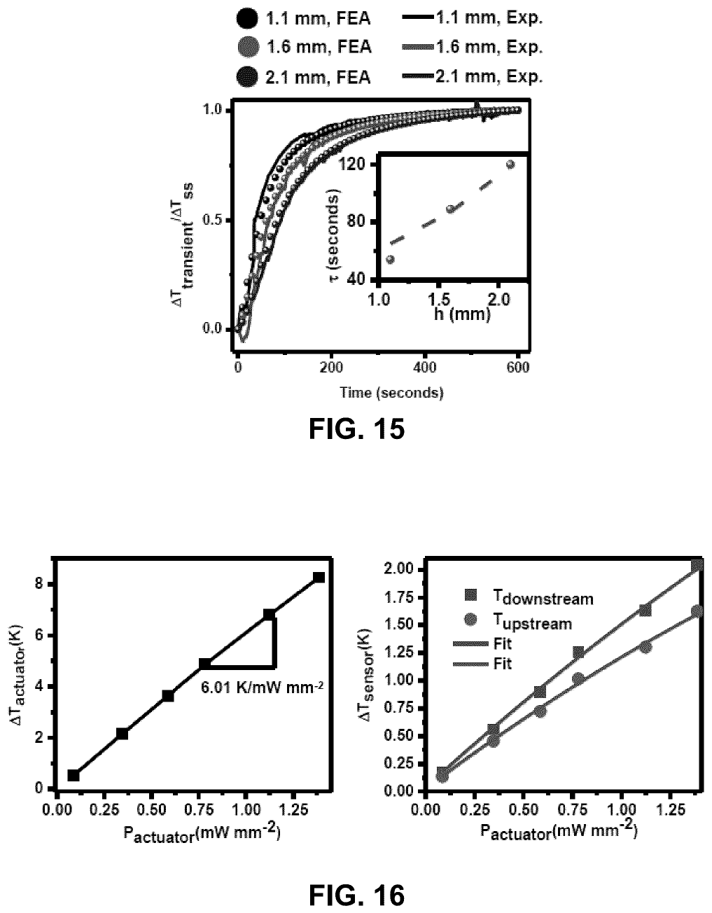

The thickness of the skin (hₛₖᵢₙ) represents an important geometric parameter. As shown in FIGS. 3D-3E, increasing hₛₖᵢₙ decreases the effects of flow on the sensor responses, simply due to the finite depth of penetration of the thermal field. Although transient techniques can be used to determine hₛₖᵢₙ from thermal measurements, as shown in FIG. 15, in practice, hₛₖᵢₙ can be measured directly using CT and Doppler ultrasound, as discussed subsequently.

The power/area of the actuator (P_{actuator}) represents an important design consideration. Increasing P_{actuator} improves the signal to noise ratio (S/N) of the measurements, but biological considerations set an upper limit for non-invasive use. The effects of P_{actuator} on S/N appear in FIG. 3F, where the signal is an averaged measurement over 60 s (measured at 5 Hz) of ΔTₛₑₙₛₒᵣₛ/T_{actuator} for a flow rate of 0.13 mL min⁻¹. The noise is the standard deviation (σ₆₀ₛ) computed to three significant digits. At sufficiently high values of P_{actuator} (P_{actuator}>1 mW/mm²) the advantages of increased actuation power diminish, and the noise stabilizes at 2% of the measured signal. The increase in local temperature varies linearly with P_{actuator} at a rate of 6.01 K (mW mm⁻²)⁻¹ on PDMS (Sylgard 184), as shown in FIG. 16.

A possible confounding effect for the measurement follows from blood flow through superficial veins, as shown in a benchtop model in FIGS. 3G-3H, for two skin thicknesses and in two configurations: flow aligned with (+x, co-flow) and opposite to (−x, counter-flow) flow of CSF flow, for rates at the upper end of the range typically encountered in veins located near the surface of the skin of the neck. In practice, co-flow represents the most realistic case, as venous blood flow typically proceeds from the brain towards the heart. Arterial flow can be neglected since its depth (>1 cm) occurs below the limit of detectability for the sensors reported here. In experiments, flow through the catheter is 0.13 mL min⁻¹, and the phantom blood vessel (Rᵥₑₛₛₑₗ=1 mm) resides (d_{blood}) 5 mm from the central axis of the sensor, and 2.5 mm from the edge of the actuator, as an extreme case. In this system, hₛₖᵢₙ is the same for both the catheter and the blood vessels. The counter-flow cases result in a 20% reduction in both ΔTₛₑₙₛₒᵣₛ/T_{actuator} and Tₛₑₙₛₒᵣₛ/T_{actuator}, while the co-flow case results in a measured reduction of <5%.

The thermal conductivity (kₛₖᵢₙ) and diffusivity (αₛₖᵢₙ) of skin also represent unknowns, with human skin exhibiting a range of 0.2 W m⁻¹K⁻¹<kₛₖᵢₙ<0.45 W m⁻¹K⁻¹ and 0.9 mm²s⁻¹<αₛₖᵢₙ<0.17 mm²s⁻¹ [11]. Phantom skins with properties that bound this range can be constructed from silicone materials with two different formulations (Sylgard 170 and Sylgard 184, Dow Corning, Inc.). Measurements of the thermal properties of these materials (FIG. 3I) match literature values: k₁₈₄=0.18∓0.01 W m⁻¹K⁻¹, α₁₈₄=0.11∓0.03 mm²s⁻¹ and: k₁₇₀=0.42∓0.01 W m⁻¹K⁻¹, α₁₇₀=0.18∓0.01 mm²s⁻¹. The measured values of ΔTₛₑₙₛₒᵣₛ/T_{actuator} are nearly identical for these two cases, as shown in FIG. 3J. By contrast, the increased rates of thermal transport associated with Sylgard 170 increases the cooling effect of the fluid, thereby reducing the values of Tₛₑₙₛₒᵣₛ/T_{actuator} as shown in FIG. 3K. The result increases the sensitivity of the sensor.

Ventricular catheters are constructed from standard medical-grade silicones, and their thermal properties are assumed to be known a-priori (k_{catheter}=0.22 W m⁻¹K⁻¹, α_{catheter}=0.12 mm²s⁻¹) [16].

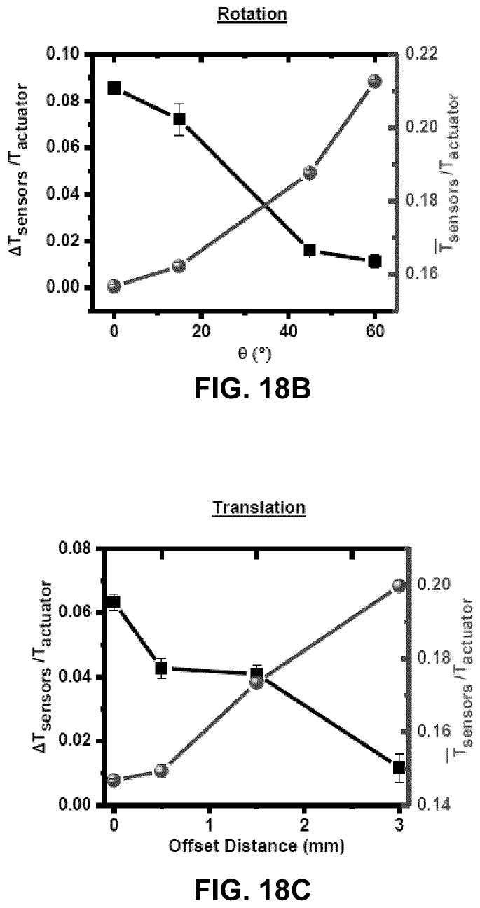

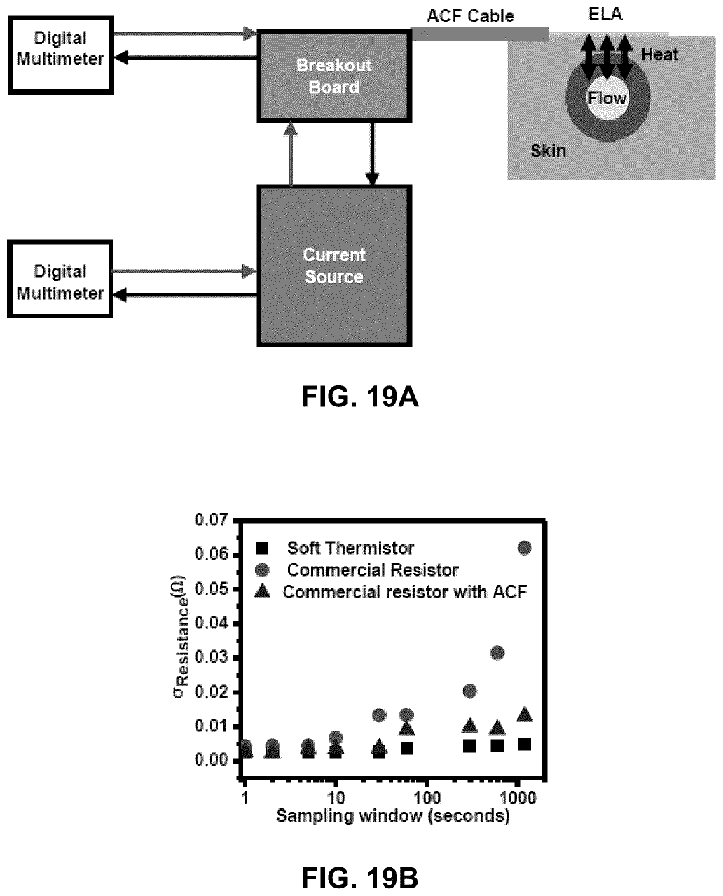

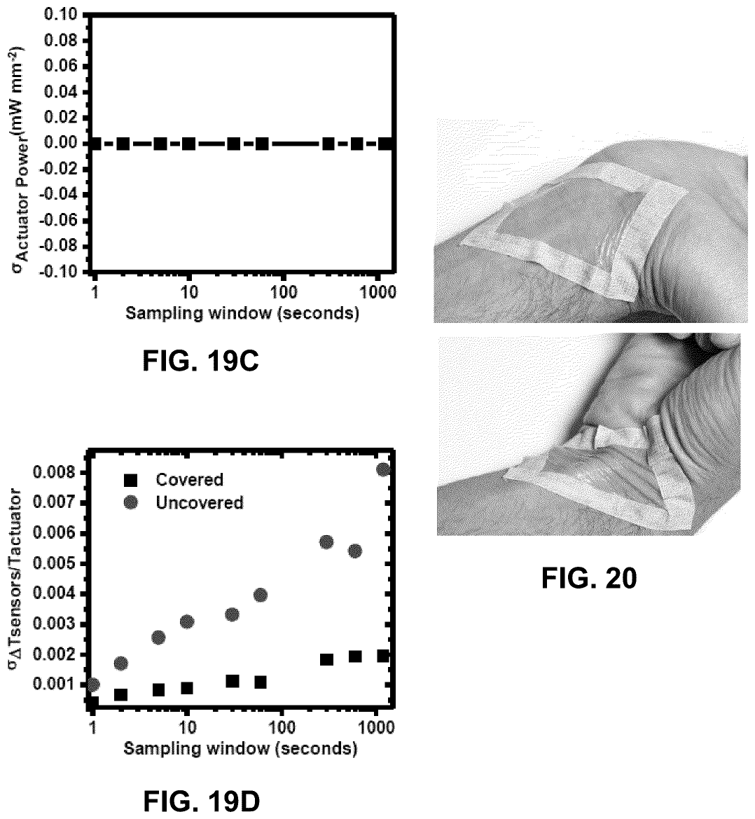

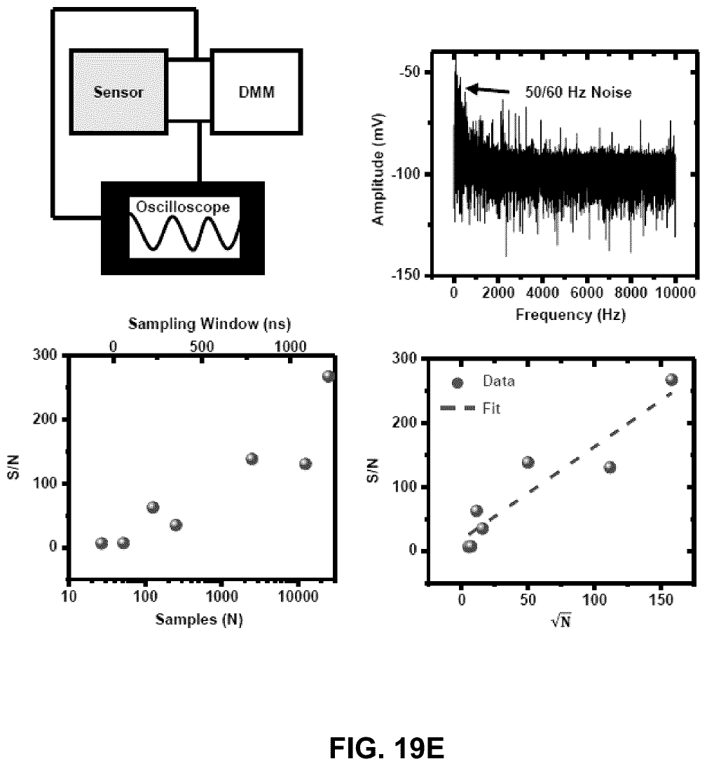

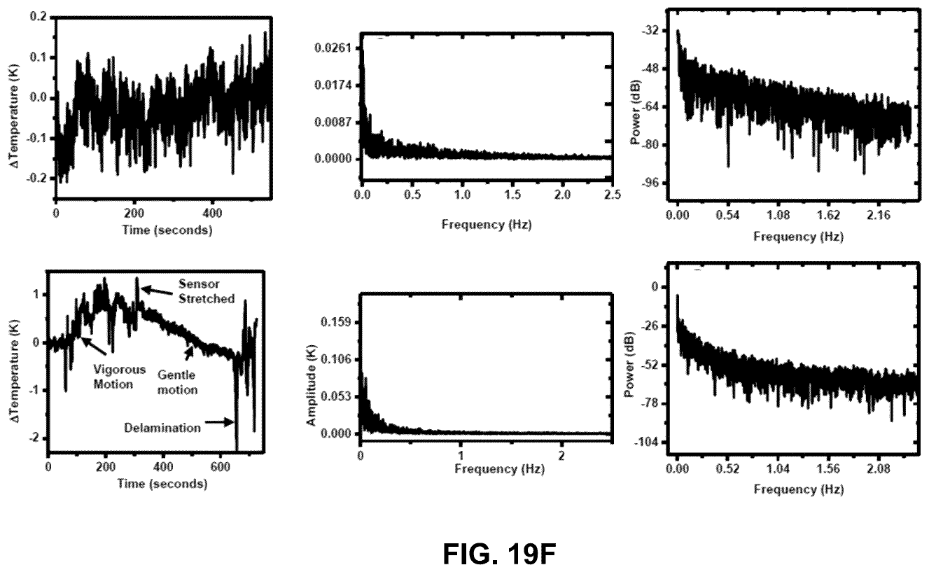

Additional experiments quantify the convective heat transfer coefficient (H_{conv}=20 W m⁻²K⁻¹, FIG. 17), tolerance in positioning (300 rotational tolerance, FIG. 18B, ˜ 1 mm translational tolerance, FIG. 18C) and noise introduced by the data acquisition system as a function of sampling window (FIGS. 19A-19D).

Systems provided herein are compatible with wireless data acquisition, including via Bluetooth. This represents an important patient care aspect, as the patient need not be hard-wired to any instruments. In this manner, continued monitoring is possible without confining patient location or motion.

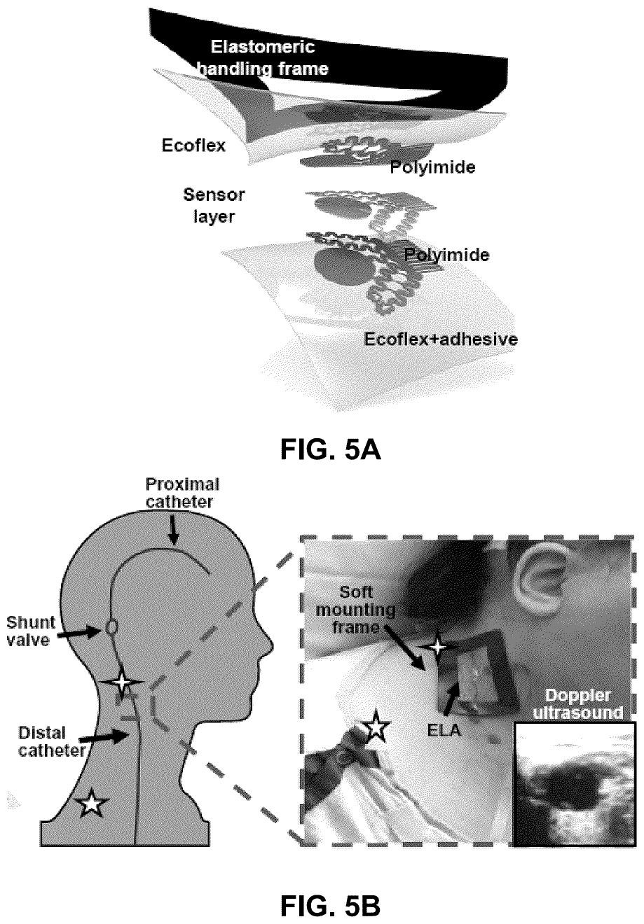

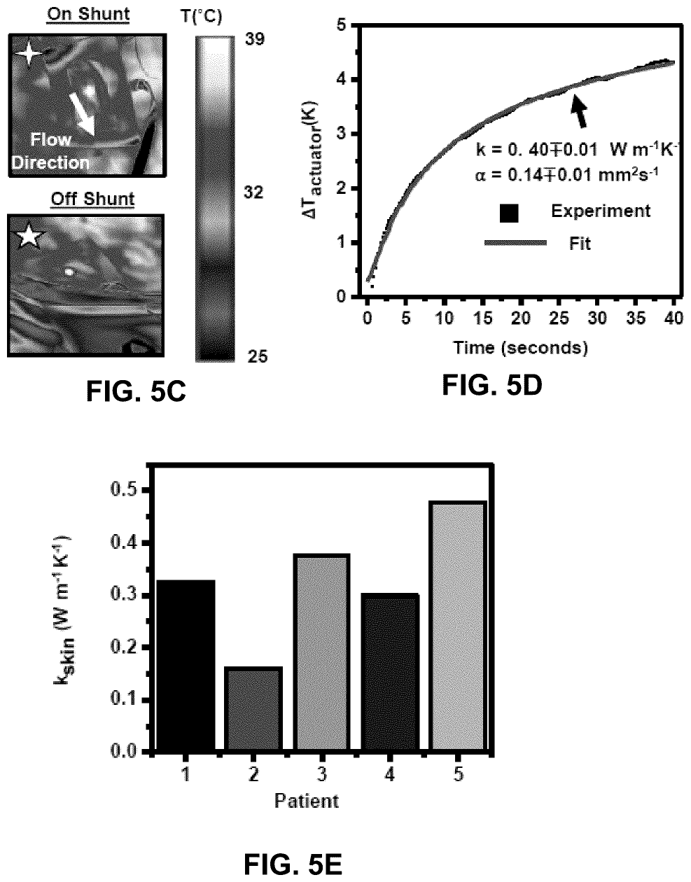

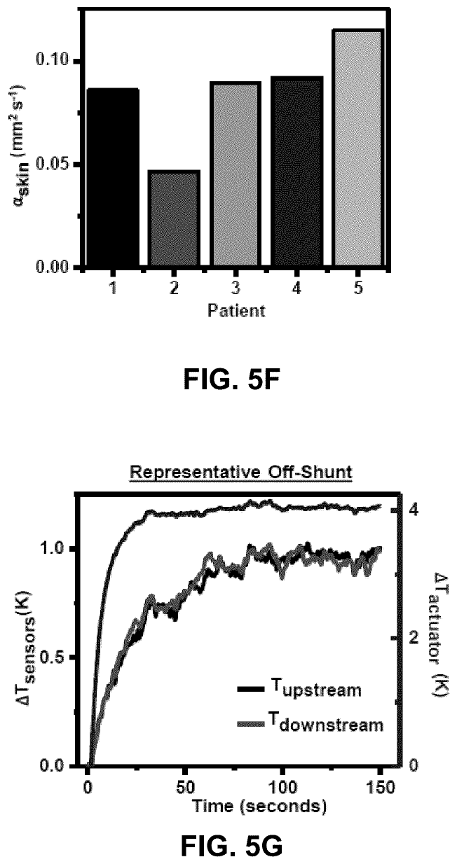

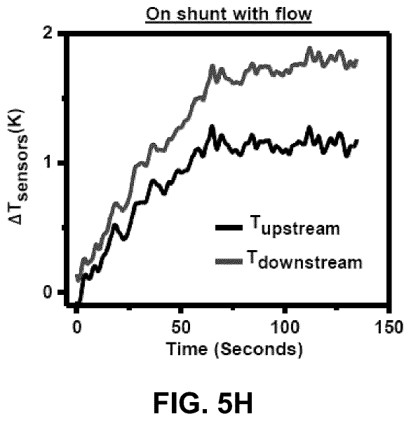

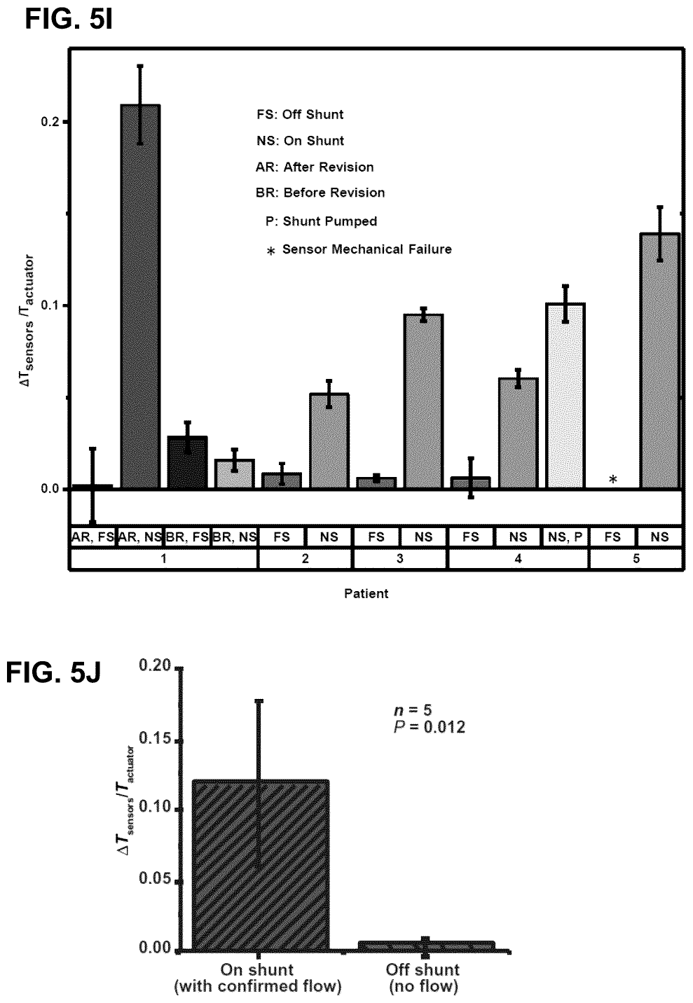

Human studies for the evaluation of ventricular shunt function: Experiments on five shunt recipients with varying pathologies demonstrate the utility of these measurement platforms. The device designs address three needs: (1) ease of handling for the surgeon to ensure facile placement and removal, (2) comfort for the patient during application, operation and removal, and (3) robust mechanical and thermal coupling to the skin. A schematic illustration of the resulting embodiment appears in FIG. 5A, showing the ELA and ultrathin elastomer substrate (100 μm, Ecoflex+MG7 1010 Adhesive) supported by an elastomeric frame (2 mm, Sylgard 170). These platforms adhere robustly and non-invasively to the skin via van der Waals interactions alone, without the need for separate adhesive layers, as illustrate in FIGS. 19A-19D, where a device maintains conformal contact with the skin even under extreme deformations. Successive measurements involve placement on the skin over the distal catheter (‘on-shunt’), and at a location adjacent to the distal catheter (‘off shunt’). The off-shunt measurement has two key uses: (1) it serves as a control for comparison to the on-shunt measurement and (2) it allows for the measurement of skin thermal properties without the influence of flow. FIG. 5B schematically illustrates the on-shunt and off-shunt location. Locating the catheter under the skin via touch was facile, and precise positioning was achieved with Doppler ultrasound (Sonosite Inc., Bellevue WA). A representative Doppler image of the catheter appears in FIG. 5B (inset). Linear markings on the device, visible in FIG. 5B, allow for easily alignment of the central axis of the actuator and sensors with the underlying shunt. Although the shunt is not visible under the skin, its ends can be easily aligned to the markings on the device via touch. Low-power actuation (1.3 mW/mm²) ensures maximum temperature increases of <5° C., as confirmed by IR images in FIG. 5C. These values are well below the threshold for sensation, in accordance with IRB-approved protocols. Markers in FIG. 5B identify mounting locations in FIG. 5C. The results show a characteristic tear-drop distribution of temperature, consistent with flow.

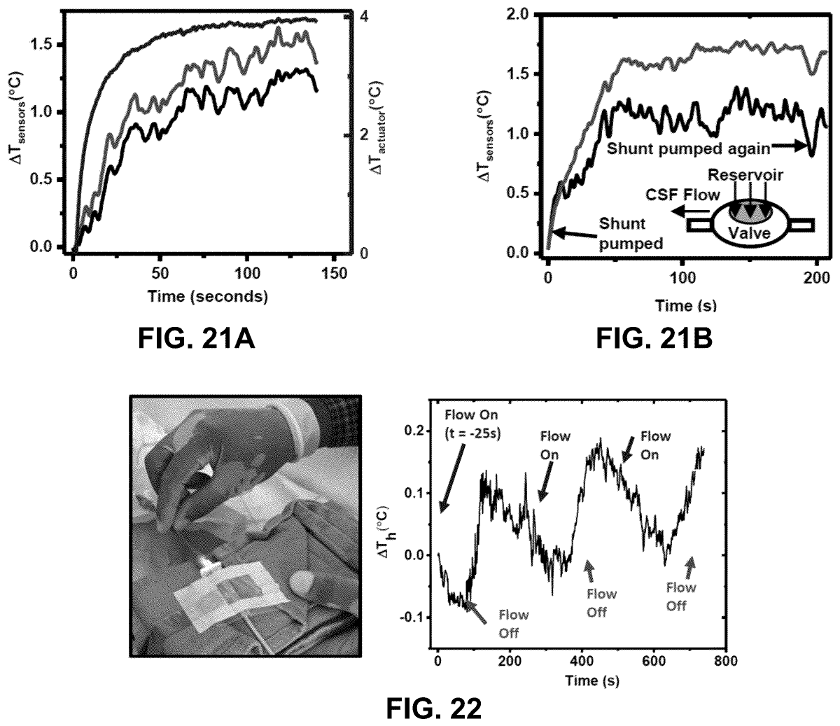

Transient, off-shunt measurements of T_{actuator} define the thermal transport properties of the patient's skin. A representative response before, during and after actuation appear in FIG. 5D. Values of kₛₖᵢₙ and αₛₖᵢₙ extracted from these data appear in FIGS. 5E-5F; the magnitudes are comparable to those expected for skin [11]. Data from the flow sensor are in FIGS. 5G-5H, where the red, black and blue curves represent the temperatures measured from the upstream sensor, the downstream sensor, and the actuator, respectively. Locations adjacent to the shunt that are free of near-surface blood vessels present no sources of thermal anisotropy and, therefore, can serve as control measurements. Results from a representative case are in FIG. 5G, where the upstream and downstream responses are nearly identical. Anisotropy that results from flow through a shunt appears in FIG. 5H. In a simple binary sense, the presence or absence of flow corresponding to shunt functioning or failure can be immediately determined simply by observing the presence or absence of thermal anisotropy. Measured values of ΔTₛₑₙₛₒᵣₛ/T_{actuator} appear in FIG. 5I for on-shunt and off-shunt locations for all 5 patients. Anisotropy appears clearly for all working shunts. Error bars correspond to standard deviations computed over 100 samples. Raw data from two additional cases appear in FIG. 20. Details of each patient's etiologies and results are in FIGS. 21A-21B.

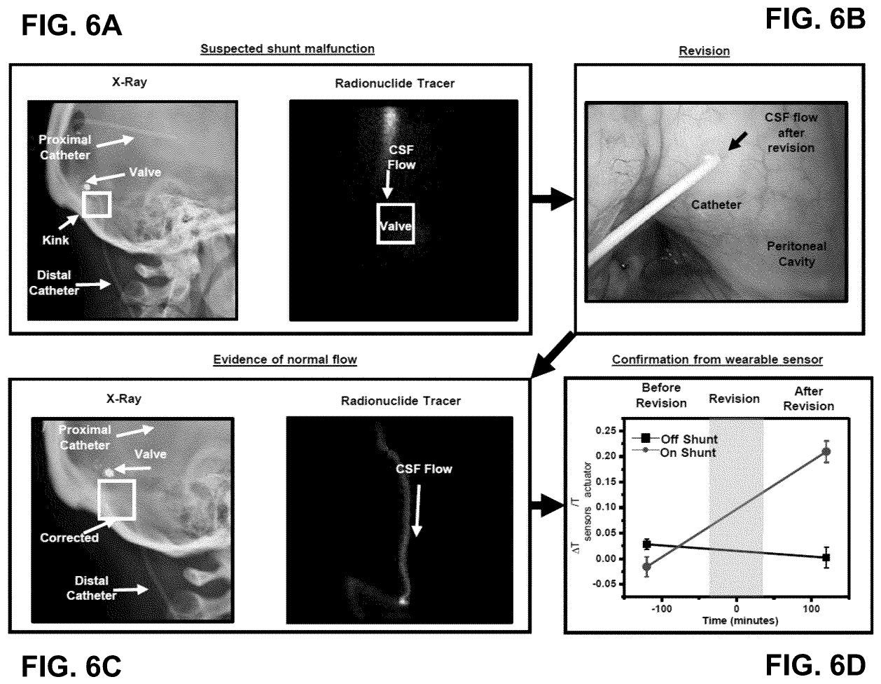

Studies by X-Ray, MRI and CT imaging validate the measurements. FIG. 6A corresponds to a patient (F, 36) with a shunt malfunction suspected to be due to a kink in the distal catheter, and later confirmed by the X-Ray and Radionuclide Tracer (RT) images. Surgical intervention relieved the kink, causing a dramatic, visible increase in flow, as shown in the optical image in FIG. 6B. The continuous presence of flow was further confirmed via post-operative X-Ray and RT, revealing a straightened distal catheter and a clear trace beyond the valve, as shown in FIG. 6C. Placement of the ELA at on- and off-shunt locations respectively, revealed no flow before the revision, consistent with X-Ray and RT imaging. Post operatively, the off-shunt measurement showed no appreciable changes, while the on-shunt measurement showed the clear presence of flow, as shown in FIG. 6D.

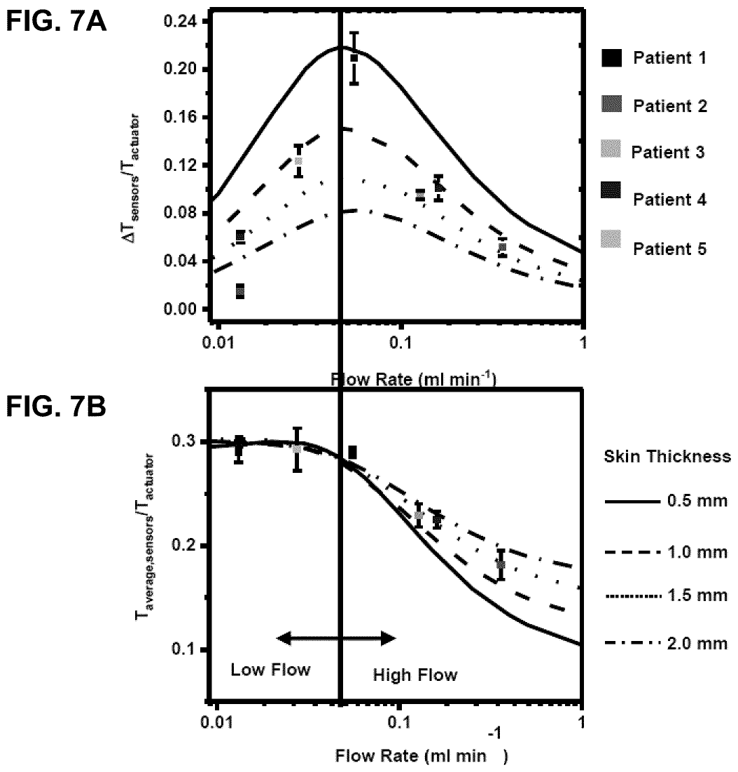

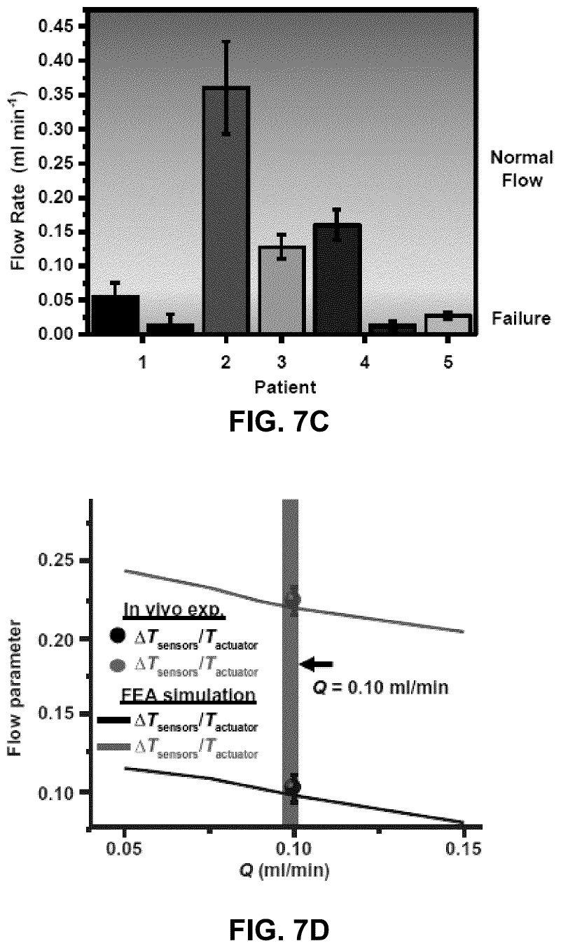

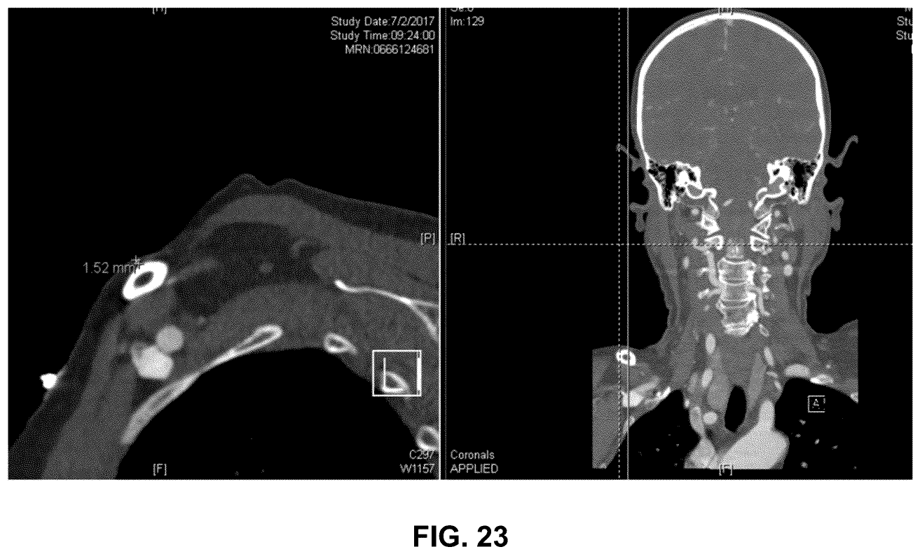

The quantitative extraction of flow rates from such data can be accomplished via fitting to FEA models that use measurements of kₛₖᵢₙ and αₛₖᵢₙ, ΔT sensors/T_{actuator} and Tₛₑₙₛₒᵣₛ/T_{actuator} and a priori knowledge of the inner and outer diameters of the catheter, and its thermal properties k_{catheter} and α_{catheter}. Placing the sensor at distal catheter locations that are determined, via touch, to be the most superficial maximizes the precision of the measurement. Analysis of CT and Doppler ultrasound images, such as the ones shown in FIG. 5B and FIG. 23 define hₛₖᵢₙ at these locations to be 1.5 mm∓0.1 mm. FEA yields computed curves for Tₛₑₙₛₒᵣₛ/T_{actuator} and ΔT sensors/T_{actuator} for a 0.01 ml min⁻¹<Q_{CSF}<1 ml min⁻¹. In this way, measured values of Tₛₑₙₛₒᵣₛ/T_{actuator} define regimes of flow, i.e. high-flow (Q>0.05 mL min⁻¹) or low-flow (Q<0.05 mL min⁻¹). Specifically, values of T̅ₛₑₙₛₒᵣₛ/T_{actuator}>0.29 represent low-flow, and T̅ₛₑₙₛₒᵣₛ/T_{actuator}<0.29 represent high-flow for all skin thicknesses, as shown in FIG. 7B. Measured values of T̅ₛₑₙₛₒᵣₛ/T_{actuator} and ΔT sensors/T_{actuator} are then iteratively fitted to yield a unique flow rate. Following this process for our measured data yields quantitative flow values.

Applications on human subjects illustrate this process. Assessments of Patient 1 prior to corrective surgery, and as in FIGS. 6A-6D, indicated a shunt malfunction, consistent with ELA measurements (0.01∓0.01 mL min⁻¹). Measurements after a surgical revision revealed a flow rate of 0.06∓0.02 mL min⁻¹. Patients 2 and 3 were not suspected of shunt malfunction and exhibited flow rates of 0.36∓0.04 mL min⁻¹ and 0.13∓0.02 mL min⁻¹ respectively, well within established ranges for healthy CSF flow [13]. Patient 4, was initially measured to have occluded flow (0.013∓0.002 mL min⁻¹). This patient had experienced severe and prolonged constipation for the past week and clinically deteriorated due to a likely pseudo-obstruction. Long term constipation can decrease the resorptive ability of the peritoneum due to increased intraabdominal pressure and a decreased pressure gradient from ventricle to peritoneum [17]. After administering a rigorous bowel regimen, the patient's mental status improved, and a subsequent measurement revealed healthy flow (0.16∓0.02 mL min⁻¹). Patient 5 was suspected to have shunt malfunction, and thermal measurements revealed highly occluded flow (0.027∓0.005 mL min⁻¹, which was later surgically confirmed. (For these studies, the sensors were not used to make clinical determinations). In patients 4 (pre-bowel examination) and 5 (pre-surgery), the results of the measurements were blinded to the physician assessment. These results appear in FIG. 7C.

Error, Noise and Uncertainty: Data analysis requires conversion of measured resistances from two sensors and one actuator, first into temperature, and then into a flow rate. A simplified schematic of this process appears in FIG. 18A. The first conversion relies on a precise, high-resolution (10 mΩ) measurement of resistance performed with a digital multimeter at a sampling frequency of 5 Hz. The inherent noise in the resistance measurement is 4.8 ppm over a 20-minute sampling window, as measured with a commercial, 1 kΩresistor and shown in FIG. 18B. The addition of a conducting anisotropic thin film (ACF) cable increases the noise to 12.5 ppm. Introducing the soft temperature sensing element and a second ACF connection further increases the noise to 93.9 ppm. Conversion to temperature relies on a linear calibration with R²>0.999, corresponding to a temperature resolution of 15 mK. Actuation involves a high-performance constant current source that exhibits remarkably stable operation, with deviations of 1.73 ppm over a 20-minute sampling window, as shown in FIG. 18C. Taken together with the effects of skin thickness and in-plane heat dissipation, the total noise in measurements of ΔT sensors/T_{actuator} are ˜2%, as shown in FIG. 18D. In practice, strains on the mechanically mismatched ACF cable and soft bond pads induced by patient motion are the primary source of noise, which we measure in vivo, on average, to be 9-10% of the measured ΔT sensors/T_{actuator} signal for all patients (on on-shunt locations). Elimination of ACF cable, either through wireless embodiments or through thin, soldered connections suggest straightforward ways of mitigating these effects.

Comparison to recent technologies: A commercially available sensor (ShuntCheck) offers an alternative to imaging-based diagnostic tools [18-21]. The system comprises a cooling pack that is held against the skin over the distal catheter, with conventional, bulk temperature sensors attached to the skin downstream, along the direction of the catheter. The pack cools flowing CSF, thereby decreasing the temperature of the downstream sensor. Although this system has high specificity (˜100%)[20] and sensitivity (80%), it suffers from key limitations. First, the embodiment is bulky and offers a poorly coupled sensor-skin interface that demands the use of a large pack (2.5 cm×2.5 cm) and significant cooling. This requirement, together with a conventional, large-scale data acquisition (DAQ) system, decreases the usability of the system and prevents continuous, long-term measurements. Second, the measurements are semi-quantitative, without an ability to account for key factors such as skin thickness, skin thermal properties and device layout. Taken together, these factors lead to overall patient discomfort and prevent straightforward interpretation of data[20]. A comparison of existing diagnostic techniques is in FIG. 16.

Implications for the treatment of hydrocephalus: The skin-like, precision sensor systems introduced here have the potential to represent a paradigm shift in clinical diagnostics of shunt malfunction. Compared to radiographic imaging, invasive sampling, and ice-pack cooling, these platforms are unique in their integration of precision, soft, thermal sensors with wireless transmission capability. By exploiting advanced concepts in the measurement of thermal anisotropy and skin-conformal epidermal electronics, these devices can provide further quantitative modes of use beyond opportunities afforded by the embodiments studied here.

Clinically, shunted individuals suffer from prolonged and costly hospital observations, exposure to toxic radiation, painful procedural interventions and discrepancies in socioeconomic care. The current standard of care is a disservice to this vulnerable population, and a better method of diagnosis would be invaluable in the management of hydrocephalic patients. The technology introduced here will introduce capabilities in personalized medicine to the hydrocephalus landscape, currently only embodied by types and settings of generalized valve systems in current treatment. By quantitatively assessing CSF flow rates, baseline flow rates can be established for individual patients after initial surgeries and at follow-up, thereby providing new insights into a patient's hydrodynamic physiology. With an abundance of prior literature describing neurosurgical exploration and witnessed intraoperative flow, such results can shed insights into the levels of flow needed to generate symptoms in vivo. Further, wireless capabilities allow ventricular shunts to be monitored telemetrically, with mobile application development aiding clinical assessment for treating physicians. As value-based healthcare transforms medical environments, precision measurements and seamless wireless transmission will provide an economically practical, clinically effective tool for the clinician. Additionally, the psychological burden of non-specific symptoms creates significant anxiety for patients, families and caregivers. With careful validation, a sensor platform of the type introduced here can be employed in at-home diagnostics, mitigating uncertainty.

Research Applications: Many poorly understood conditions stem from neurological hydrodynamic dysfunction, including normal pressure hydrocephalus (NPH), idiopathic intracranial hypertension (IIH), and slit-ventricle syndrome. These conditions cause tremendous suffering for affected patients. NPH, characterized by a triad of neuropsychiatric changes, urinary incontinence, and gait imbalance, may affect up to 20 million (typically elderly) individuals annually. The associated pathophysiology may be related to choroid villous malabsorption, and overdrainage in these individuals may cause venous rupture and subdural hematoma, often necessitating neurosurgical intervention [Kameda, Lesniak]. IIH predominantly affects younger female patients and has been linked to abnormal Vitamin A metabolism and intracranial venous stenosis. The comparatively diminutive ventricular systems possessed by these patients lead to high risks of shunt malfunction stemming from ventricular collapse, complicating revision surgical attempts and leading to extended, painful hospital stays [McGirt, Karsy, Liu]. Similarly, slit-ventricle syndrome patients experience poor ventricular wall compliance, with malfunctions largely undetectable in radiographic study [Drake]. By understanding individual flow rates in each of these conditions, novel and improved treatment approaches can be developed for their care. Ultimately, personalized, better-designed shunt systems, with integrated flow monitoring systems, will offer the ability to appropriately compensate for these physiological flow patterns, providing hope to a population with significant need.

Fabrication of the sensor system: For the sensors presented here, fabrication began with spin-casting a sacrificial layer of poly(methyl methacrylate) (700 nm) onto a 4″, undoped Si-wafer. A dielectric layer, polyimide (PI, 3 μm) is then spun on. For the epidermal linear array (ELA), a single bilayer film of Cr/Au 10/100 nm deposited by electron-beam evaporation onto the wafer, and patterned by photolithography and etching formed the sensors and serpentine interconnects, in accordance with design rules in stretchable electronics [22-25]. For the epidermal square array (ESA), a bilayer film of Cr/Au 10/100 nm was photolithographically defined to form 100 resistive temperature sensing elements, arranged in a 10×10 array, around a central resistive thermal actuator. A multilayer film of Ti/Cu/Ti/Au 20/600/20/25 nm evaporated and photolithographically defined yielded a first layer of rows of serpentine interconnects to address each row of sensors. Photolithography and reactive ion etching (RIE defined via holes in a second, spin-cast layer of PI (3 μm). A second multilayer film of Ti/Cu/Ti/Au 20/600/20/25 nm formed using the same methods as the first, defined columnar serpentine interconnects to address each column. For both the ELA and ESA designs, spin-casting defined a final layer of PI layer (3 μm) also patterned in the geometry of the metal traces. A final RIE step isolated the outline of the device and opened via holes for wired connections to external data acquisition electronics. Immersion in an acetone bath undercuts the sacrificial PMMA layer, allowing for release and transfer via water soluble tape. The devices were then transferred to a thin, bi-layer silicone membrane (Ecoflex, 20 μm, Dow Corning, MG 7 1010 Skin Adhesive, 20 μm), spin-cast onto a glass slide. Immersion in warm water dissolved the tape, and a spin-cast top layer of silicone (Ecoflex, 50 μm) completed the device. A thin (100 μm), double-sided sheet adhesive (JMS 1400, Label Innovations, Ontario, Canada) was laser structured to form an outline around the device. This sheet adhered to the silicone and a handling frame, either in the form of a printed circuit board containing wireless transmission electronics, or a simple, thick, elastomeric frame to facilitate handling of the wired electronics. Anisotropically conducting films (ACF) established connections to wired data acquisition electronics. The sensor resistances were then calibrated to temperatures measured by IR imaging.

Fabrication of Flexible Printed Circuit Boards: Fabrication began with a commercially available, dense, tri-layer Cu/PI/Cu laminate (Pyralux, 6535, DuPont, 18 μm/75 μm/18 μm). Laser structuring (LPKF U4, LPKF Systems, Germany) patterned conducting traces and bond pads, with a resolution of 50 μm. Commercially available SMD resistors, capacitors, along with a Bluetooth microcontroller (NRF 52, Nordic Semiconductor) and battery, in addition to the soft electronic components were bonded to the PCB via reflow soldering.

Data Acquisition Systems: Data were recorded from the ELA resistive elements via digital multimeters (DMM) (NI, USB 4065, National Instruments). Actuation power was supplied with a constant current source (Keithley 6220, Tektronix). The ESA requires a voltage output module (NI 9264, National Instruments) that sequentially actuates each of the ten input channels with 3V, and a single-channel DMM to measure current. A red LED connected in series with each channel served as a visual indicator of multiplexing and the status of each addressed channel. A mechanical REED relay module (J-Works, 2418, J-Works Inc.) was used to time multiplex measurements from each of the ten channels. All data were recorded via custom software designed and programmed in LabView (National Instruments), and processed with custom algorithms in Matlab (Mathworks Inc., Natick, MA).

### Thermo-Mechanical Modeling and Finite Element Analysis

Benchtop Experiments: A phantom skin assembly was constructed for in-vitro evaluation. A distal shunt catheter (Medtronic, Minneapolis, MN) was embedded in a matrix of PDMS (Sylgard 184, Dow Corning) supported by a 3D printed mold containing struts to preventing sagging of the catheter. Optical images of this assembly appear in FIGS. 13A-13C. The depth of the catheter under the PDMS was 1.1 mm, as seen in FIG. 13B, and the sensor was laminated onto the free surface of the assembly, as shown in FIG. 13C. The catheter was connected to a syringe pump, through which flow rates were varied to yield the experimental data shown in FIGS. 3A-3K. Water was chosen to be the test fluid for these experiments. The observed flow rates were 33% higher than values displayed on the pump, which was corrected for via a simple, linear calibration, where true flow values were measured with precise weight measurements on an analytical balance at fixed time intervals. The syringe pump was allowed to come to steady state at a flow rate for 180s before the measurement was made. Each measurement consisted of a 60s “off” period with Q_{actuator}<0.001 mW/mm², followed by a 600 s actuation (“on”) period, with Q_{actuator}=1.45 mW/mm², followed by a 180 s off period to return the sensor back to its baseline, pre-actuation temperature value. Simultaneously, thermographs were recorded with an IR Camera (FLIR Systems, a6255sc), with a high-magnification lens. Different skin-thicknesses were achieved by casting PDMS onto 3D printed molds with negative relief structures with defined heights and laminating the resulting sheets onto the fluidic assembly described above.

Human Study Design: Patients were recruited from an existing ICU population. The inclusion criterion was any patient with an implanted ventricular shunt, regardless of whether they were suspected of shunt malfunction. Patient 1 (36, F) presented with a Pseudotumor cerebri and suspected shunt malfunction that was then surgically corrected. Patient 2 (F, 53) presented with a Chiari I malformation, and was not suspected of shunt malfunction. Patient 3 (M, 32), presented with a Glioblastoma multiform, with no suspected malfunction, and Patient 4 (F, 58) presented with a Glioblastoma multiforme with suspected pseudoobstruction due to acute and prolonged constipation that was resolved with a rigorous bowel examination. Patient 5 (F, 30) presented with suspected malfunction due to obstruction, with severely diminished but non-zero flow, which was confirmed during surgery. Depending on the clinical condition of the patient, they were either asked to either sit at 45° or completely supine. A single measurement consisted of placing the sensor on the skin and waiting for 60 s for the sensor to equilibrate with the skin. Low power thermal actuation (1.6 mW/mm²) was then supplied for 240s, and then halted for the next 120s, while making continuous temperature measurements of both the sensors and the actuator. All data recording occurred at 5 Hz and processing used an adjacent-averaging filter with a 10-point sampling window. Two successive measurements each were made on skin directly overlying the shunt, and at a skin location adjacent to the shunt. The shunt was easily located, and alignment marks on the device allowed for easy alignment. An elastomeric enclosure around the device facilitated handling of the device.

### Table 1. Table Summarizing Etiology of, and Measurements Made on Each Patient.

TABLE 1
Table summarizing etiology of, and measurements made on each patient.

| | | | | | Flow | Flow | | |
|---|---|---|---|---|---|---|---|---|
| | | | | | Detected | Detected | | |
| | Underlying | | | Malfunction | (pre- | (post- | Imaging | Skin |
| | Condition | Age | Sex | Present | intervention) | intervention) | Correlate | Irritation |
| 1 | Pseudotumor | 36 | F | Y | N | Y | Y¹ | N |
| | cerebri | | | | | | | |
| 2 | Chiari I | 53 | F | N | Y | N/A | N/A | N |
| | malformation | | | | | | | |
| 3 | Glioblastoma | 32 | M | N | Y | N/A | N/A | N |
| | multiforme | | | | | | | |
| 4 | Glioblastoma | 58 | F | Y | N | Y | Y² | N |
| | multiforme | | | | | | | |
| 5 | Post- | 30 | F | Y | Y | N/A³ | Y⁴ | N |
| | hemorrhagic | | | | | | | |

TABLE 2
Table summarizing existing shunt diagnostic tools and techniques.

| Modality | Cost | Time (min) | Sensitivity | Specificity | PPV | NPV |
|---|---|---|---|---|---|---|
| X-Ray[1, 2] | 440 | 84 | 4-26% | 92-99% | 13 | 93.9 |
| CT[3-6] | 1323 | 83 | 54-80% | 80-90% | 71 | 90.8 |
| MRI[5-8] | 3239 | 115 | 40-62.8% | 84-92% | 75 | 86.5 |
| RSPS[9-12] | 750 | 45 | 47-65% | 86-92% | 71 | 71 |
| ShuntCheck[13-15]* | Unknown | 360 | 80% | 100% | 58 | 96 |

### REFERENCES

1. Xu, S., et al., Soft microfluidic assemblies of sensors, circuits, and radios for the skin. Science, 2014. 344(6179): p. 70-74.
2. Norton, J. J., et al., Soft, curved electrode systems capable of integration on the auricle as a persistent brain-computer interface. Proceedings of the National Academy of Sciences, 2015. 112(13): p. 3920-3925.
3. Kim, J., et al., Epidermal electronics with advanced capabilities in near-field communication. small, 2015. 11(8): p. 906-912.
4. Dagdeviren, C., et al., Conformal piezoelectric systems for clinical and experimental characterization of soft tissue biomechanics. Nature materials, 2015. 14(7): p. 728-736.
5. Choi, J., et al., Thin, Soft, Skin-Mounted Microfluidic Networks with Capillary Bursting Valves for Chrono-Sampling of Sweat. Advanced healthcare materials, 2017. 6(5).
6. Koh, A., et al., A soft, wearable microfluidic device for the capture, storage, and colorimetric sensing of sweat. Science translational medicine, 2016. 8(366): p. 366ra165-366ra165.
7. Tian, L., et al., Flexible and Stretchable 3ω Sensors for Thermal Characterization of Human Skin. Advanced Functional Materials, 2017.
8. Krishnan, S., et al., Multimodal epidermal devices for hydration monitoring. Microsystems & Nanoengineering, 2017. 3: p. 17014.
9. Zhang, Y., et al., Theoretical and Experimental Studies of Epidermal Heat Flux Sensors for Measurements of Core Body Temperature. Advanced healthcare materials, 2016. 5(1): p. 119-127.
10. Webb, R. C., S. Krishnan, and J. A. Rogers, Ultrathin, Skin-Like Devices for Precise, Continuous Thermal Property Mapping of Human Skin and Soft Tissues, in Stretchable Bioelectronics for Medical Devices and Systems. 2016, Springer. p. 117-132.
11. Webb, R. C., et al., Thermal transport characteristics of human skin measured in vivo using ultrathin conformal arrays of thermal sensors and actuators. PLoS One, 2015. 10(2): p. e0118131.
12. Webb, R. C., et al., Epidermal devices for noninvasive, precise, and continuous mapping of macrovascular and microvascular blood flow. Sci Adv, 2015. 1(9): p. e1500701.
13. Hidaka, M., et al., Dynamic measurement of the flow rate in cerebrospinal fluid shunts in hydrocephalic patients. European Journal of Nuclear Medicine, 2001. 28(7): p. 888-893.
14. Carslaw, H. S. and J. C. Jaeger, Conduction of heat in solids. Oxford: Clarendon Press, 1959, 2nd ed., 1959.
15. Sandby-Moller, J., T. Poulsen, and H. C. Wulf, Epidermal thickness at different body sites: relationship to age, gender, pigmentation, blood content, skin type and smoking habits. Acta Dermato Venereologica, 2003. 83(6): p. 410-413.
16. Braley, S., The chemistry and properties of the medical-grade silicones. Journal of Macromolecular Science-Chemistry, 1970. 4(3): p. 529-544.
17. Martinez-Lage, J. F., et al., Severe constipation: an under-appreciated cause of VP shunt malfunction: a case-based update. Child's Nervous System, 2008. 24(4): p. 431-435.
18. Ragavan, V. V., Evaluation of shunt flow through a hydrocephalic shunt: a controlled model for evaluation of the performance using Shuntcheck. 2017: p. 1-4.
19. Boyle, T. M., J; Neuman, M; Tamber, M; Hickey, R W; Heuer G; Leonard J; Leonard J C; Keating R; Chamberlain J; Frim D; Zakrzewski, P; Klinge, P; Merck, L; Platt, J; Bennett, J; Sandberg, D; Boop, F; Zorc, J, ShuntCheck versus Neuroimaging for Diagnosing Ventricular Shunt Malfunction in the Emergency Department. American Association of Pediatrics, 2017: p. 1-2.
20. Madsen, J. R., et al., Evaluation of the ShuntCheck Noninvasive Thermal Technique for Shunt Flow Detection in Hydrocephalic Patients. Neurosurgery, 2011. 68(1): p. 198-205.
21. Recinos, V. A., E; Carson, B; Jallo, G, Shuntcheck, A Non-invasive Device To Assess Ventricular Shunt Flow: One Institution's Early Experience. American Association of Neurological Surgeons, 2009. Abstract: p. 1-1.
22. Zhang, Y., Y. Huang, and J. A. Rogers, Mechanics of stretchable batteries and supercapacitors. Current Opinion in Solid State and Materials Science, 2015. 19(3): p. 190-199.
23. Zhang, Y. H., et al., Experimental and Theoretical Studies of Serpentine Microstructures Bonded To Prestrained Elastomers for Stretchable Electronics. Advanced Functional Materials, 2014. 24(14): p. 2028-2037.
24. Zhang, Y., et al., Mechanics of ultra-stretchable self-similar serpentine interconnects. Acta Materialia, 2013. 61(20): p. 7816-7827.
25. Zhang, Y., et al., Buckling in serpentine microstructures and applications in elastomer-supported ultra-stretchable electronics with high areal coverage. Soft Matter, 2013. 9(33): p. 8062-8070.

### Example 2: System Characterization and Use

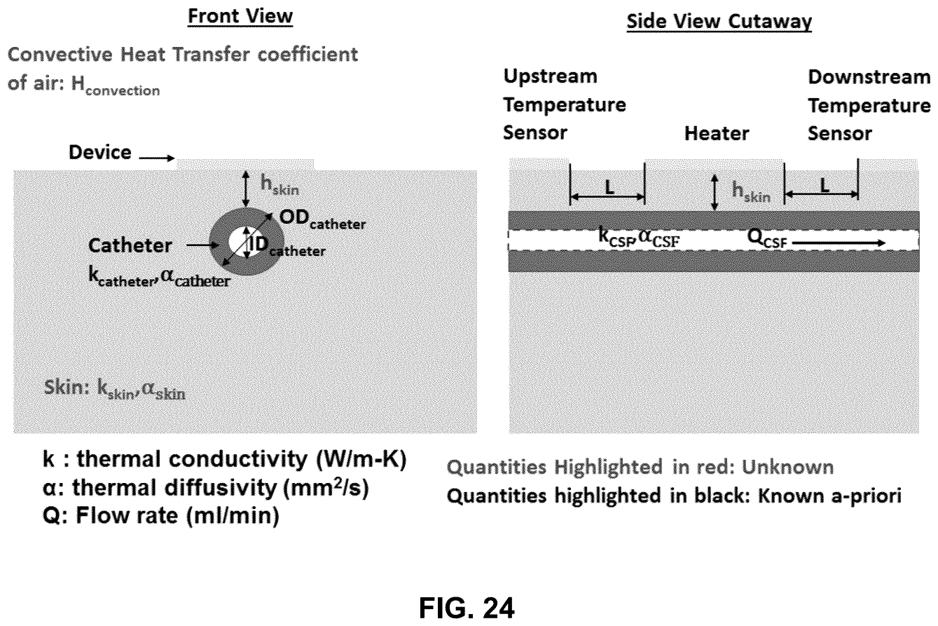

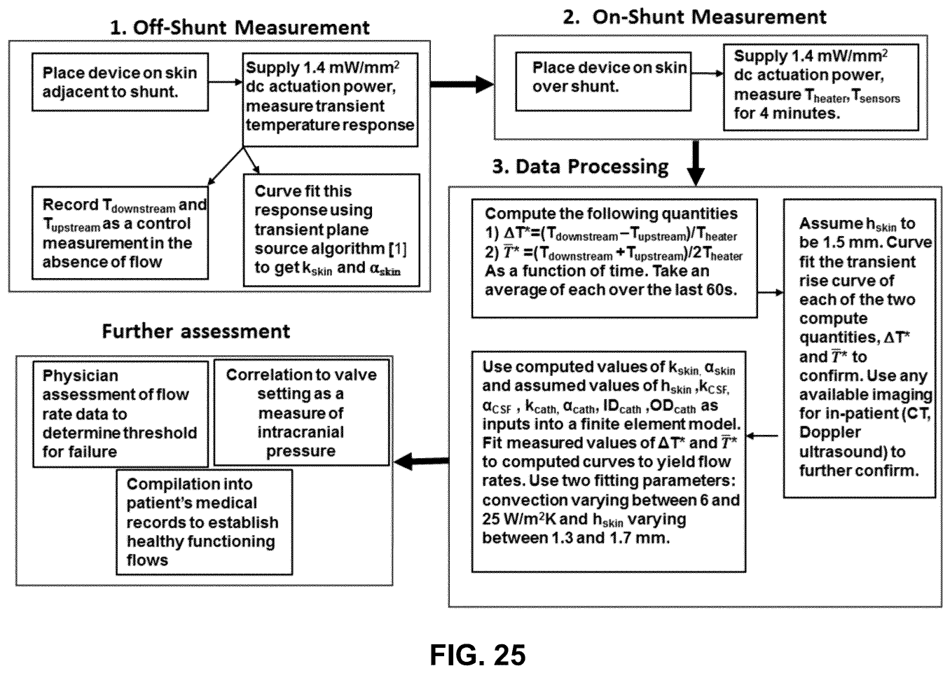

FIGS. 24-28 illustrate various features of the devices and methods described herein. FIG. 24 illustrates the position of the device and temperature sensors and various parameters used to calculate temperature and flow-rate in a subdermal conduit (described in FIG. 24 as “catheter”). Unknown quantities include skin-related parameters, such as depth of the conduit from the skin surface (hₛₖᵢₙ), as well as thermal conductivity (k) and diffusivity (α) of the skin. Known parameters include the thermal conductivity (k) and diffusivity (α) of the conduit and fluid in the conduit, outer and inner diameters of the conduit, and upstream, downstream distance between the sensors and the actuator (labeled “heater”), and temperature measured by the sensor and applied by the heater. In controlled conditions, the flow-rate (Q) of the fluid in the conduit may be known. From these parameters, flow-rate may be determined, as summarized in the flow-chart of FIG. 25.

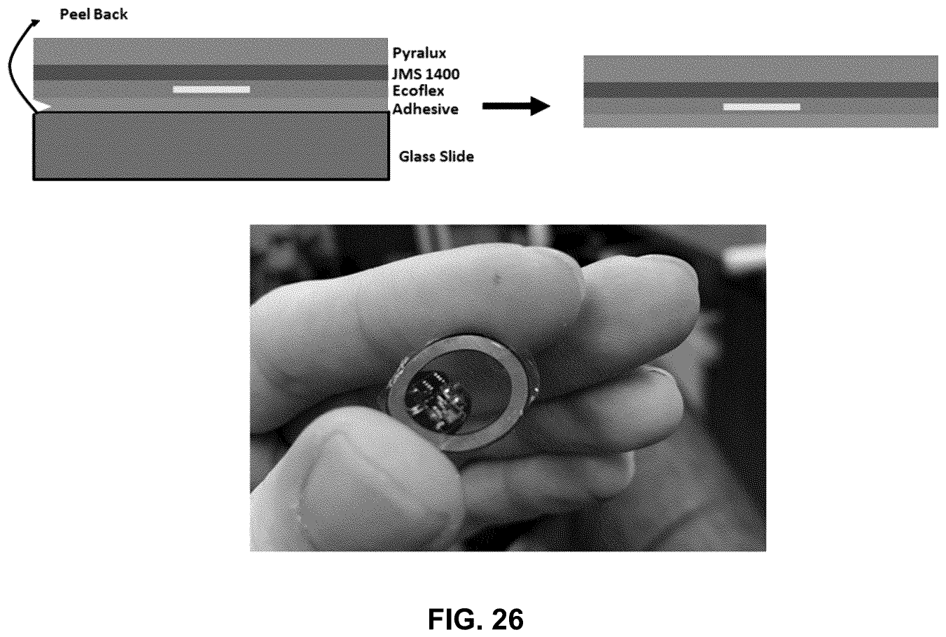

The active sensing portion of the device is extremely thin and, therefore, relatively difficult to handle. To assist with handling, any of the devices described herein may have a handle layer, including as shown in FIG. 26. A rigid layer, such as a glass slide, may support any of the devices described herein, including with an adhesive layer and encapsulation layers. The device may be peeled off the handle layer and be ready for conformal contact with the skin.

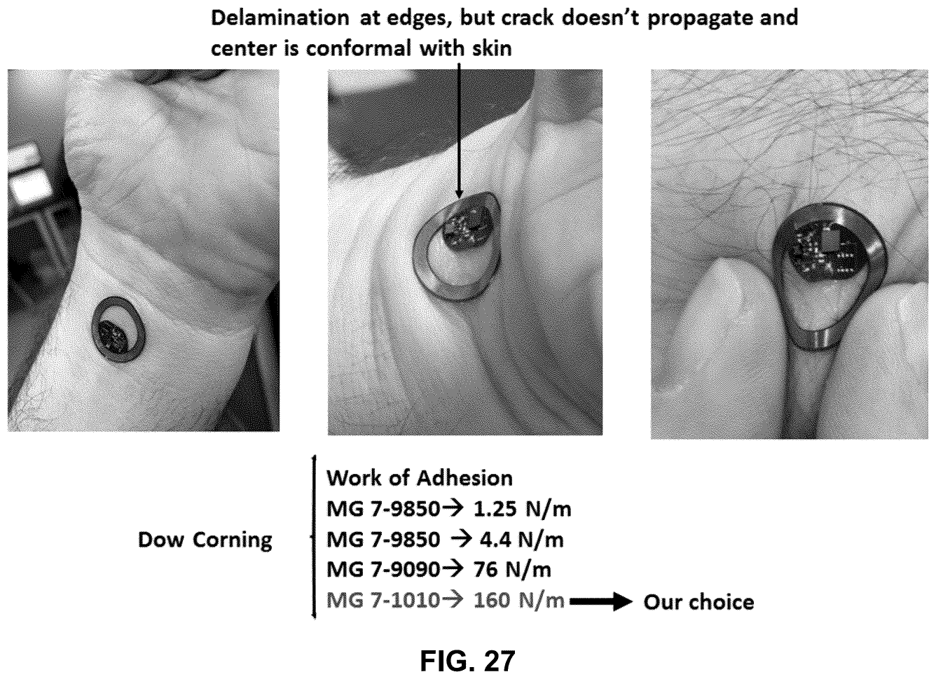

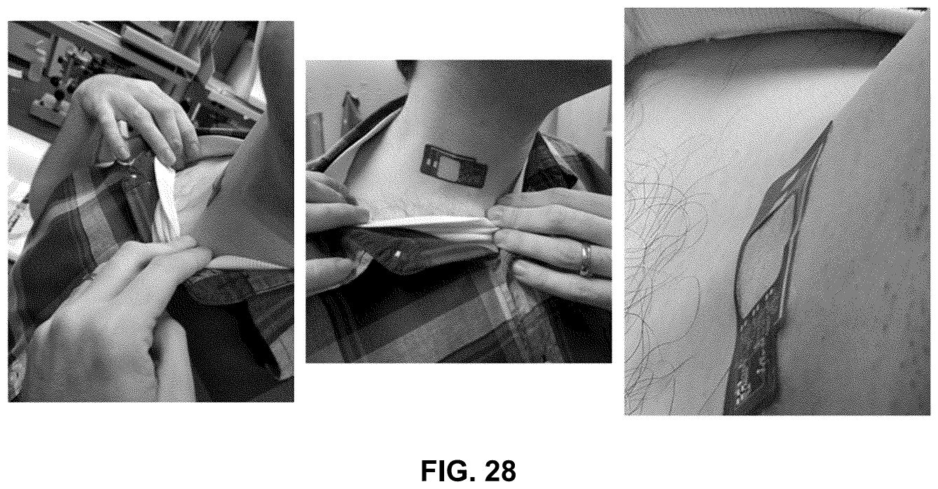

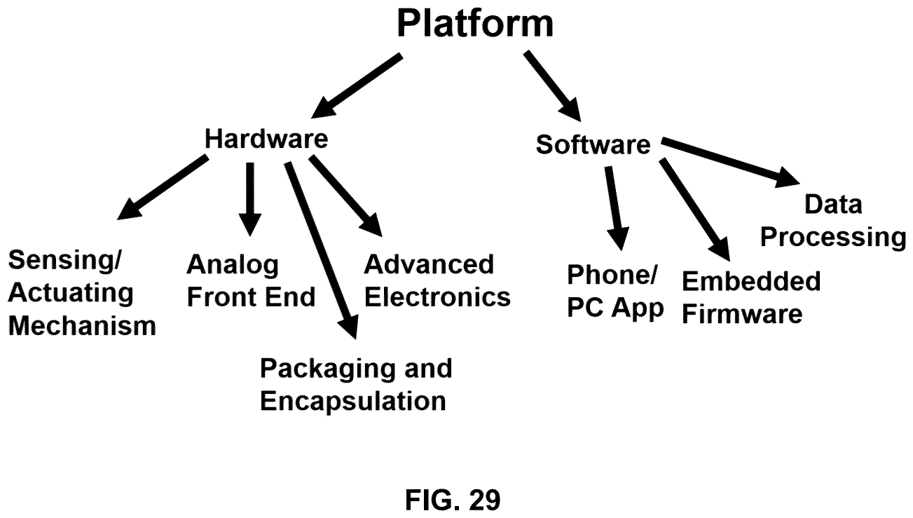

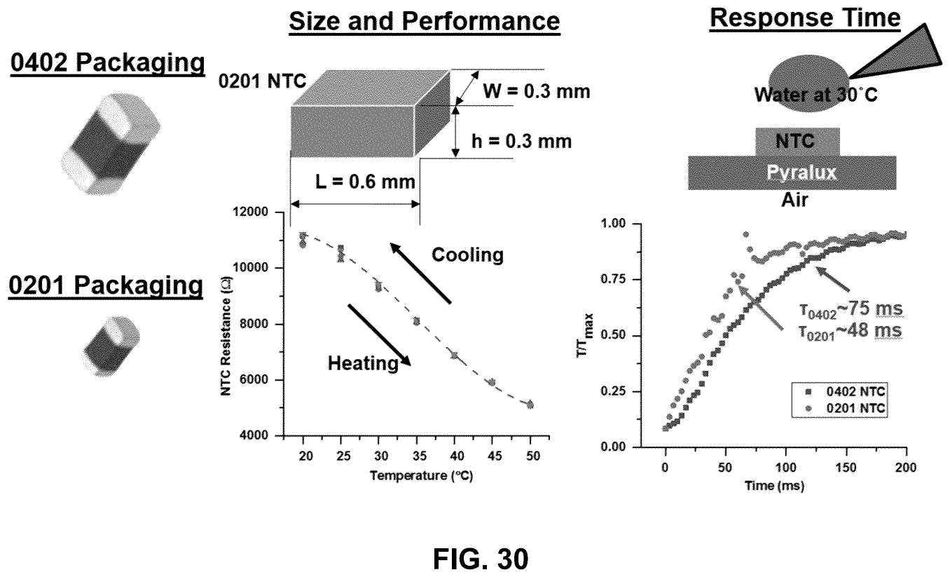

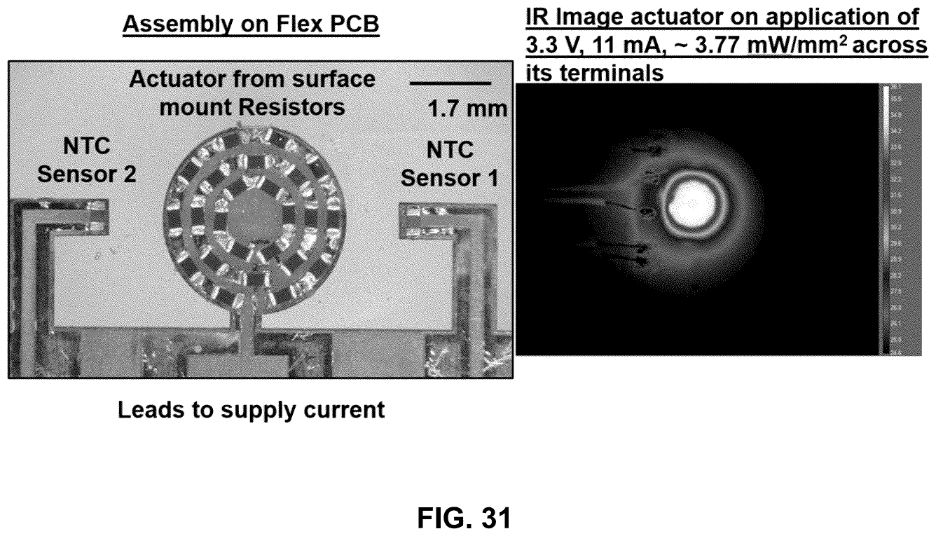

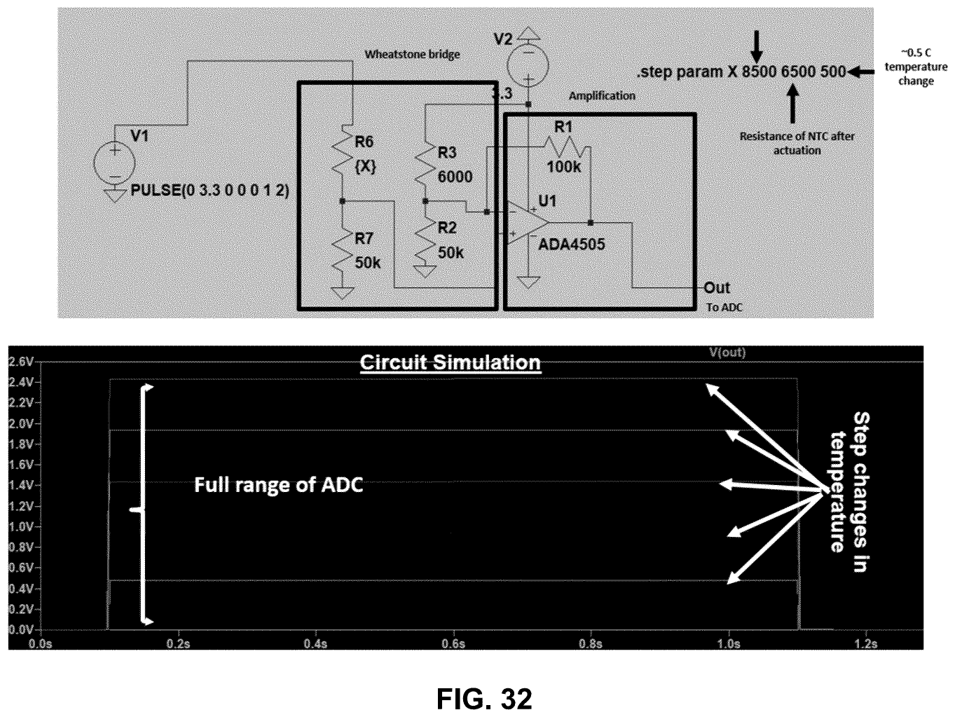

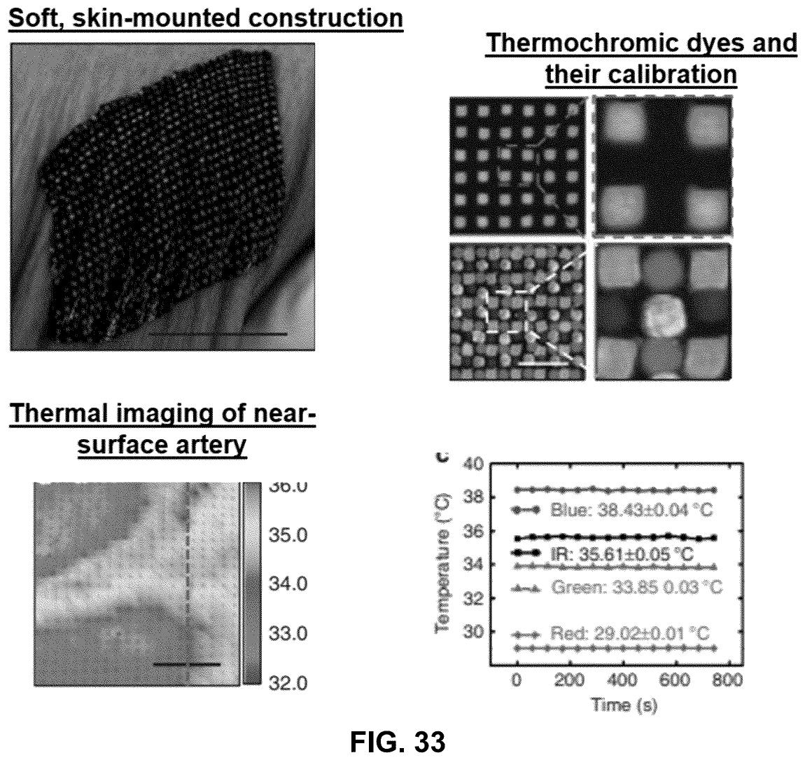

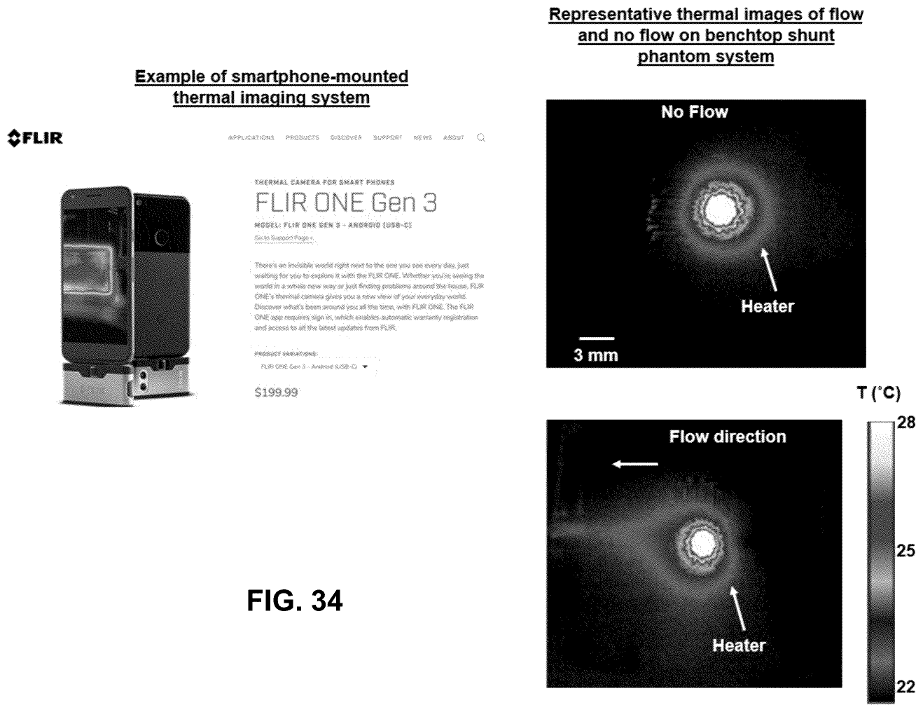

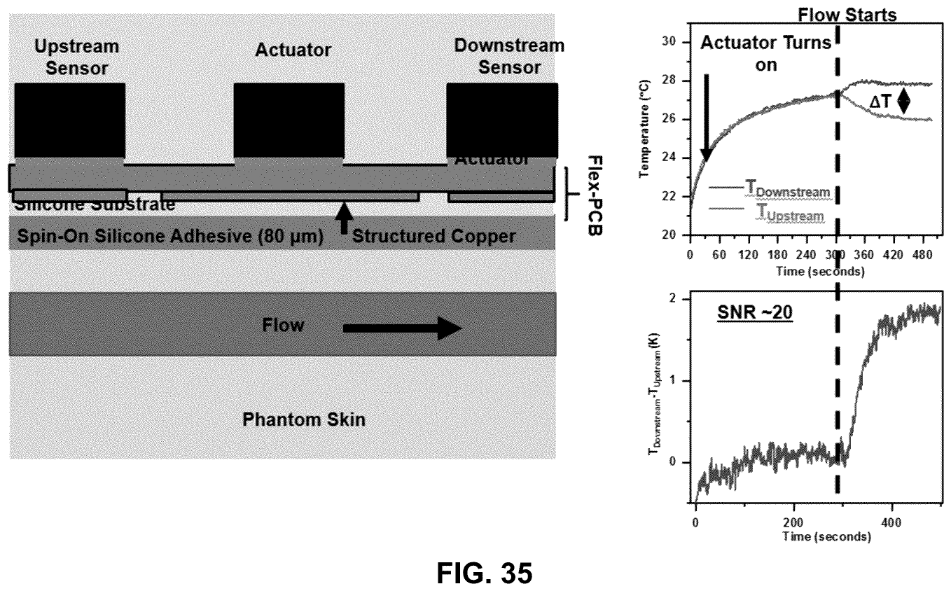

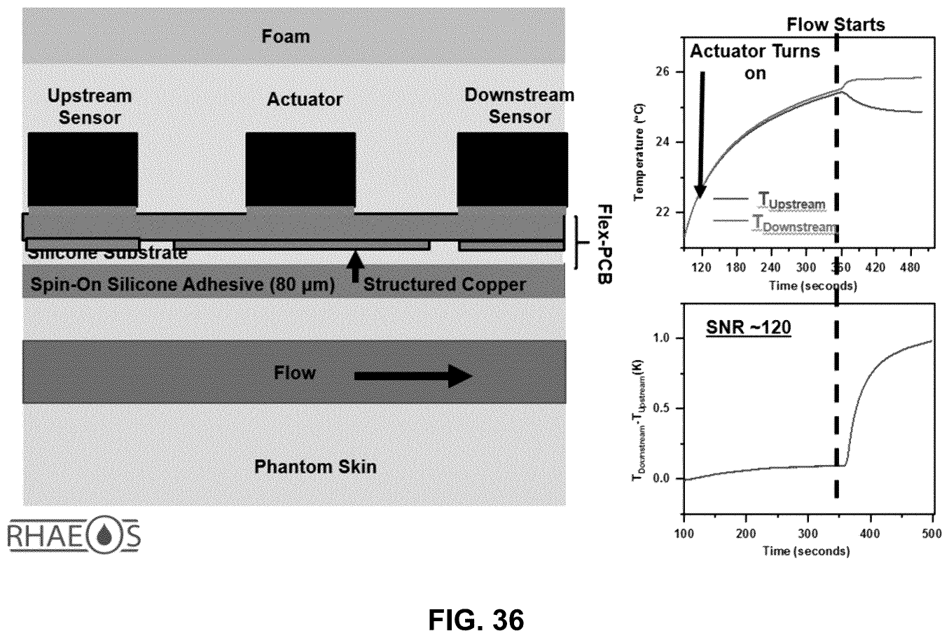

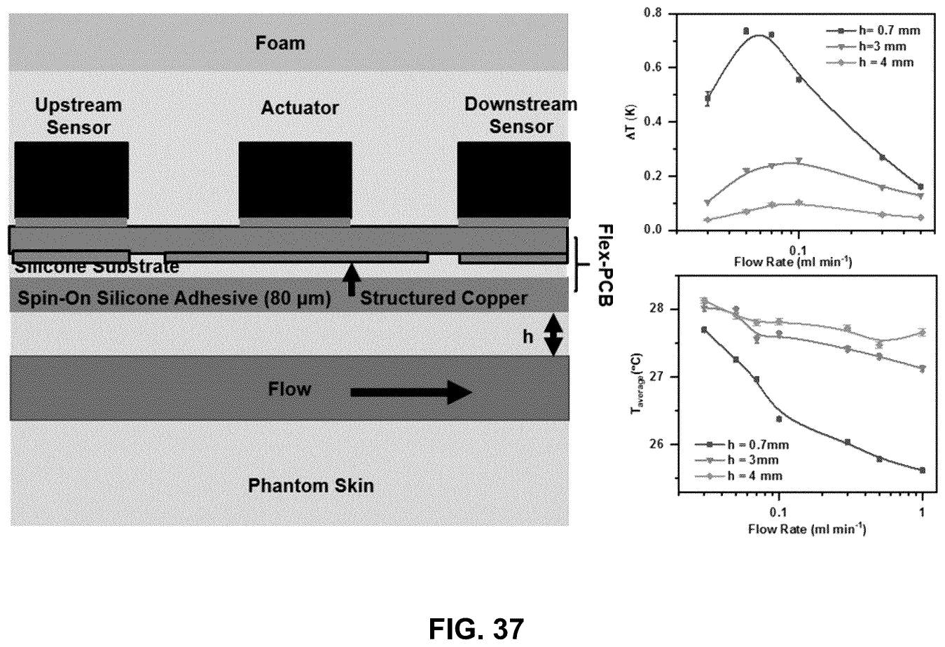

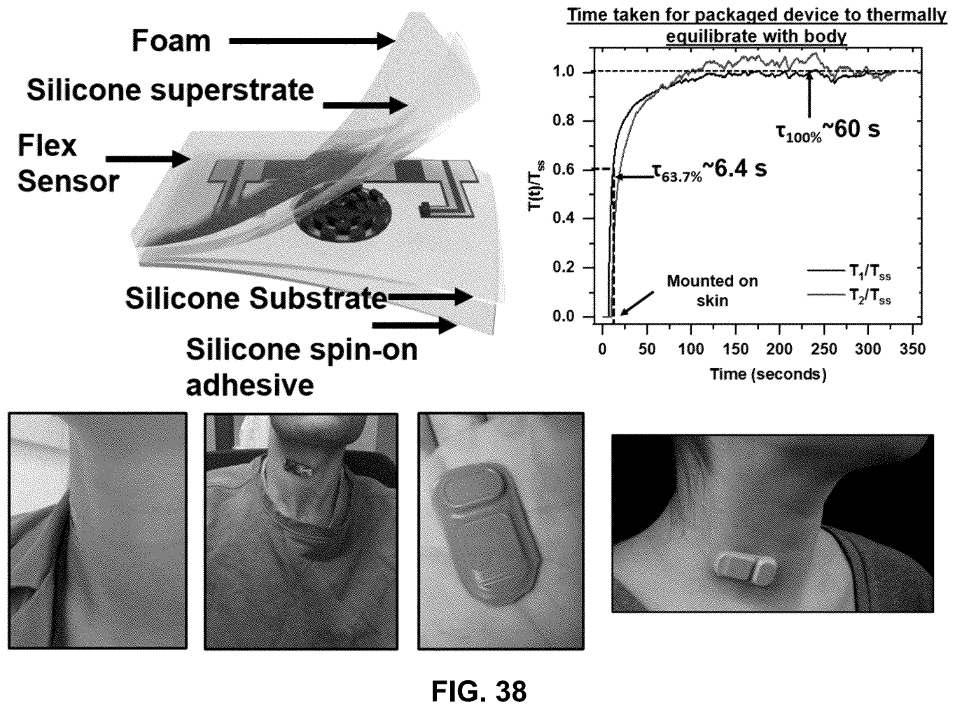

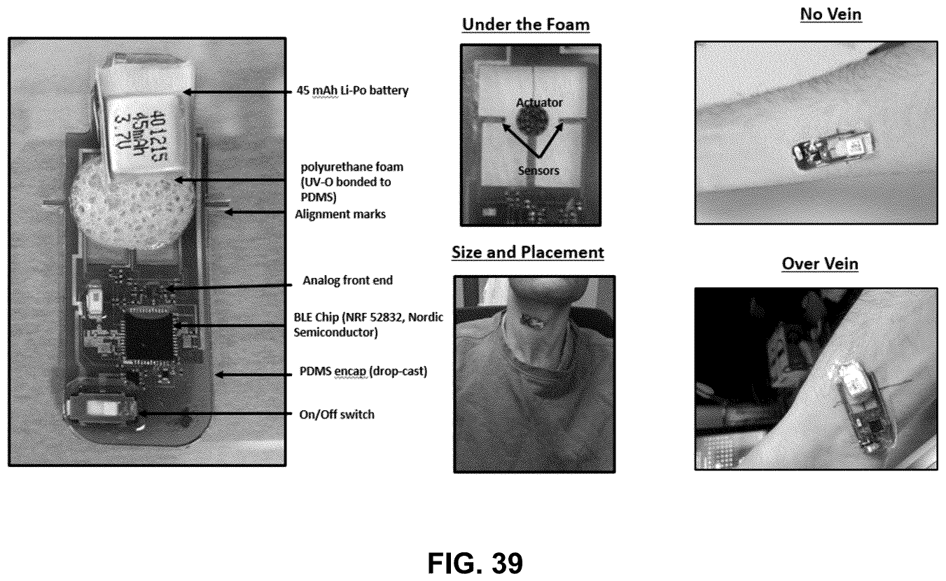

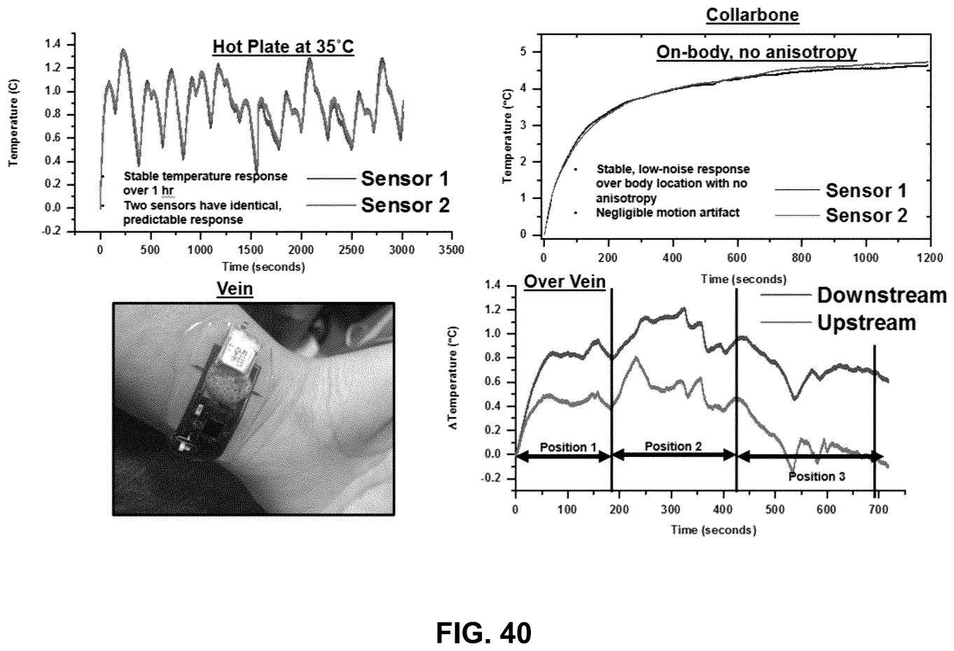

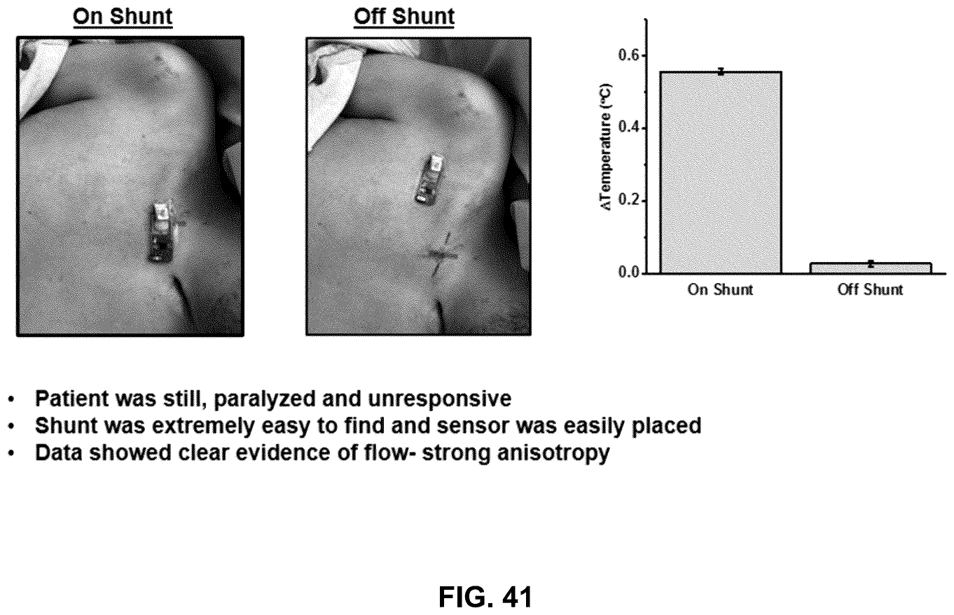

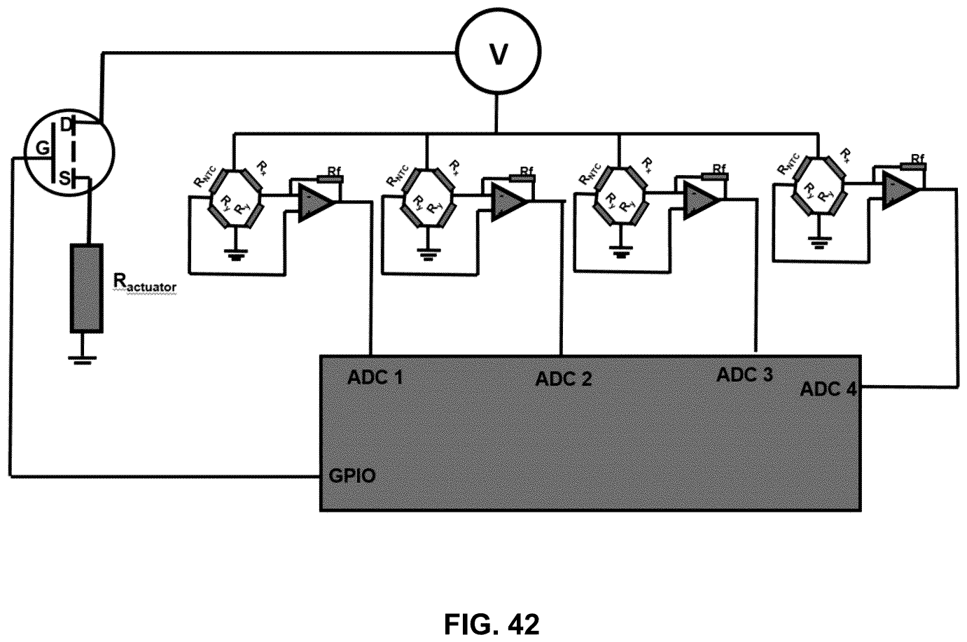

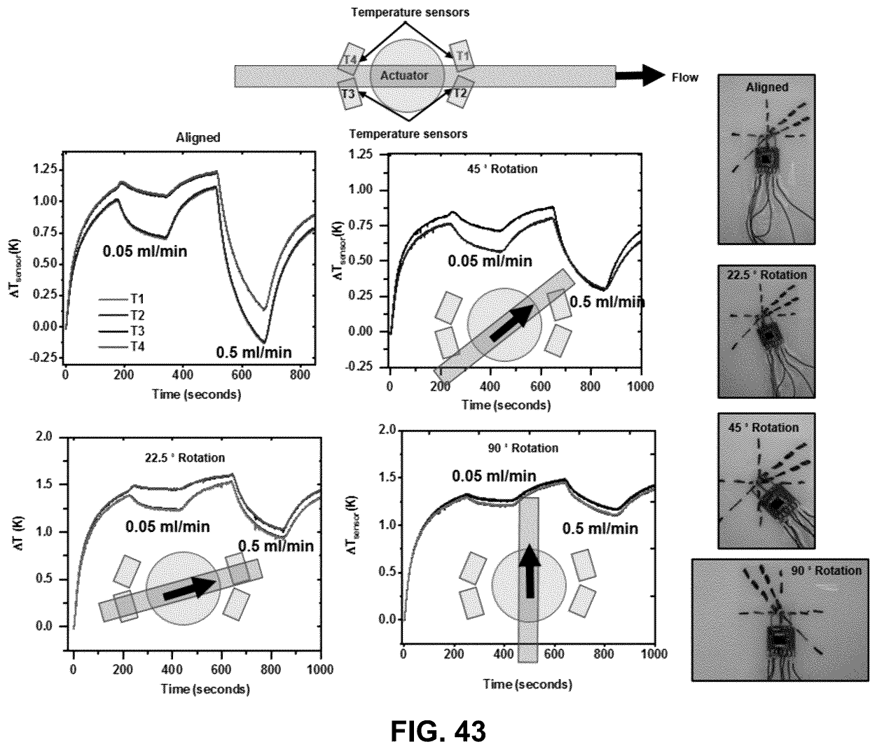

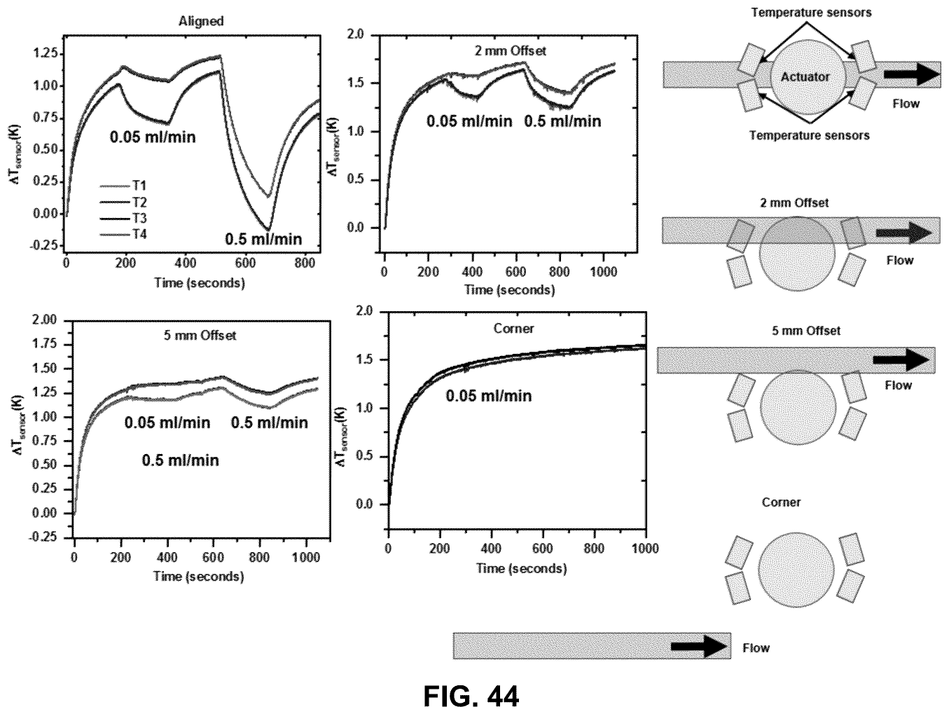

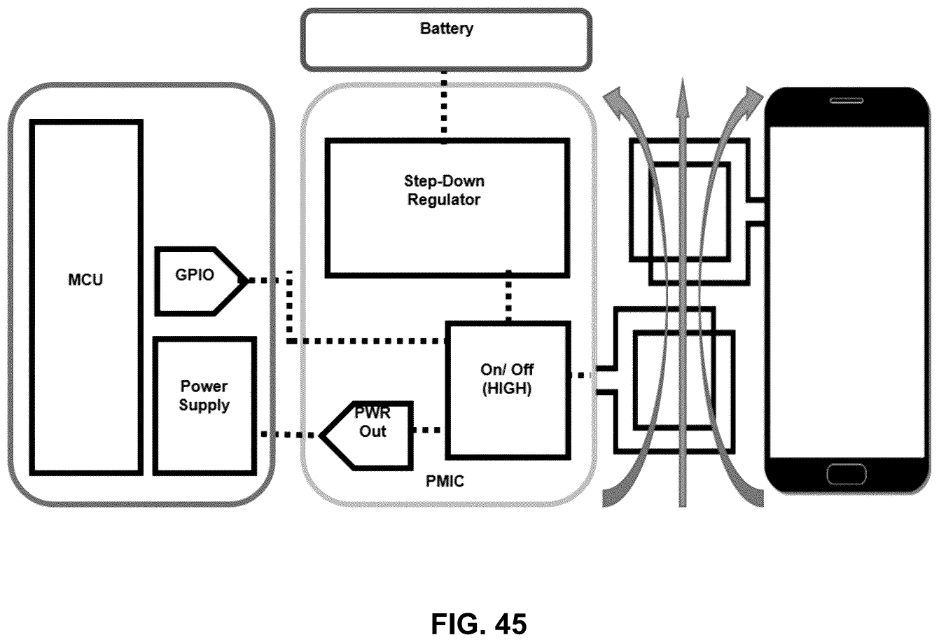

FIGS. 27-28 illustrate a carrier substrate having an open passage through a central portion of the carrier substrate, wherein the conformable device is positioned in the open passage to provide improved handling characteristics during application and durability during use. The carrier substrate in FIG. 27 is circular, and in FIG. 28 is rectangular. Any number of shapes may be used, including depending on the application and location of interest.

The sensor described herein has many potential applications in wide range of applications, including in the medical field. For example, bypass vascular grafts, and cardiac stent flow may be assessed post-procedurally. This involves placing a sensor over an applicable area with appropriate depth and quantifying flow parameters. Similarly, sensors applied over large vessels may be used to assist in detecting micro-emboli pertinent to the management of diseases such as carotid atherosclerotic disease. Finally, appropriate pressure readings may be invaluable in providing non-invasive arterial pressure measurements in the operating room or angiography suite, abrogating the need for invasive arterial line placement.

### Example 3: Soft Tissue Mounted Flow Sensors

We claim:

1. A device to measure a subdermal fluid flow parameter, comprising:
a substrate;
at least one upstream temperature sensor supported by the substrate;
at least one downstream temperature sensor supported by the substrate; and
a thermal actuator supported by the substrate and positioned between said upstream temperature sensor and said downstream temperature sensor;
wherein the upstream temperature sensor and the downstream temperature sensor are located on the same side of the substrate.

2. The device of claim 1, wherein
the temperature sensors are one or more of: diode temperature sensors, positive temperature coefficient of resistance (PTC) sensors, negative coefficient of resistance (NTC) sensors, and colorimetric temperature sensors.

3. The device of claim 1, further comprising
a controller in electronic communication with the downstream temperature sensor, the upstream temperature sensor and the thermal actuator.

4. The device of claim 3, further comprising
a microprocessor in electronic communication with the controller configured to calculate a flow parameter from the measured upstream and downstream temperatures.

5. The device of claim 4, further comprising
a wireless communication component to wirelessly connect the microprocessor to the controller.
